(19) United States
(12) Patent Application Publication
(10) Pub. No.: US 2026/0007598 A1
(43) Pub. Date: Jan. 8, 2026

(54) COMPOSITIONS, DEVICES AND METHODS FOR RESPRITORY DELIVERY OF ACTIVE PHARMACEUTICAL INGREDIENTS

(71) Applicant: HONEYWELL INTERNATIONAL INC., Charlotte, NC (US)

(72) Inventors: Erik BOLDT, Buffalo, NY (US); Gregory Laurence SMITH, Buffalo, NY (US)

(73) Assignee: HONEYWELL INTERNATIONAL INC., Charlotte, NC (US)

(21) Appl. No.: 19/259,932

(22) Filed: Jul. 3, 2025

Related U.S. Application Data
(60) Provisional application No. 63/668,096, filed on Jul. 5, 2024.
(60) Provisional application No. 63/721,633, filed on Nov. 18, 2024.
(60) Provisional application No. 63/761,287, filed on Feb. 21, 2025.

(51) Int. Cl. A61K 9/00; A61K 31/122; A61K 31/164; A61K 31/40; A61K 47/06; A61M 15/00

(52) U.S. Cl. CPC A61K 9/008; A61K 31/122; A61K 31/164; A61K 31/40; A61K 47/06; A61M 15/0015

(57) ABSTRACT

Disclosed are medicinal compositions, and devices, methods and systems which provide ipratropium bromide carried by at least 1234ze(E) and ethanol.

### CROSS REFERENCE TO RELATED APPLICATIONS

This application relates to and claims the priority benefit of each of U.S. Provisional Application 63/668,096, filed Jul. 5, 2024, U.S. Provisional Application 63/721,633, filed Nov. 18, 2024, and U.S. Provisional Application Nos. 63/761,287, filed Feb. 21, 2025. Each of the provisional applications identified in the paragraph is incorporated herein by reference in their entireties as if fully set forth below.

### FIELD OF THE INVENTION

This invention relates to compositions, systems, devices and methods for the delivery of medicament compositions through respiratory tract of the user. In particular aspects, this invention relates to pressurized metered dose inhalers (“pMDIs”) containing an active pharmaceutical ingredient (“API”) in solution and to medicinal aerosol compositions, methods and devices for delivering same from the pMDI to the respiratory tract of the user.

### BACKGROUND OF THE INVENTION

Pressurized metered dose inhalers (pMDIs) have long been used to deliver medicaments, including anticholinergics and bronchodilators, to the areas of patients needing treatment for various conditions. For example, ipratropium is used as an anticholinergic and/or a bronchodilator and has been used for the treatment of various maladies, including chronic obstructive pulmonary disease (COPD), Parkinson's disease and urinary incontinence. Beclomethasone, including beclomethasone dipropionate (BDP), is a corticosteroid that works by preventing certain cells in the lungs and breathing passages from releasing substances that cause asthma symptoms. Formoterol, also known as formoterol, is a long-acting B2 agonist (LABA) and has been used as a bronchodilator in the management of asthma and chronic obstructive pulmonary disease (COPD). Glycopyrrolate is an anticholinergic and has been used to relax muscles in the airways to improve breathing and to treat chronic obstructive pulmonary disease (COPD).

pMDIs may be used to deliver medicaments in a solubilized form or as a suspension. Typically, pMDIs use a relatively high vapor pressure propellant to carry and expel aerosolized droplets containing the API away from the pMDI and towards the respiratory tract when the pMDI is activated. The propellant/carrier for the active pharmaceutical ingredient (sometimes referred to herein as “API”) must be safe for the patients' use and be pharmaceutically acceptable. For many APIs, including but not limited to ipratropium, beclomethasone (including beclomethasone dipropionate (BDP)), formoterol, glycopyrrolate and combinations of two or more of these, the API is preferably in the pMDI as a solute in a solvent carrier. The carrier can comprise one or more materials that act as a propellant for the pMDI and as a solvent for the API.

Carrier properties can have an impact on pMDI performance. For example, the one or more materials that make up the carrier should preferably have an appropriate boiling point and vapor pressure so that it can be liquefied in a closed container at room temperature but develop a high enough pressure when the pMDI is activated to deliver the drug as an atomized composition even at low ambient temperatures. Further, the carrier should be of low acute and chronic toxicity. It should have a high degree of chemical stability with the API in solution therein and with the container and the metallic and non-metallic components of the pMDI device. The carrier should also have a low propensity to extract low molecular weight substances from any elastomeric materials in the pMDI device. Finally, the carrier should not present a significant flammability risk to the patient in use. In particular, it should form a non-flammable or low flammability mixture when mixed with air in the respiratory tract. Environmentally desirable properties, such as low GWP and low ODP, are also generally highly desirable.

pMDIs are one of the most widely used system for the delivery of drugs via inhalation. The ultimate objective of a pMDI is to accurately deliver, upon actuation by the person in need of relief, the drug to the respiratory tract of a patient using a delivery composition in which the drug is contained. Although some APIs are used by being suspended or dispersed in the carrier, the present invention relates to APIs that are dissolved in the carrier. Thus, the carrier is preferably also able to maintain the drug in a homogeneous solution.

As will be appreciated by those of skill in the art, the efficacy of a pMDI delivery system depends, in part, on the amount of the formulation, and hence the amount of the API, that is delivered to the mouth of the user upon actuation of the pMDI. Typically, a pMDI is designed to have a targeted amount of the formulation to be dispensed from the valve of the pMDI. However, applicants have come to appreciate that in many pMDIs used for the delivery of formulations containing API(s) in solution, a proportion of the formulation released from the valve of the pMDI does not reach the mouth of the user. While applicant does not wish to be bound by or limited to any particular theory of operation, it is believed that this reduction in formulation that reached the mouth of the user is due in part to the formulation impinging upon and sticking to or otherwise not proceeding into the mouth of the user. Thus, the proportion of the formulation which reaches the mouth of the user for each actuation depends on a generally unpredictable influence of many factors, including buy not necessarily limited to plume geometry, spray velocity and formulation physical properties as it exits the valve. In typical prior art pMDIs used to deliver APIs in solution, the proportion of formulation which reaches the mouth of the user is on the order of about 80% by mass.

Applicants have unexpectedly found that it is possible according to the present invention to formulate APIs in solution so as to substantially increase the proportion of the formulation which reaches the mouth of the user, and preferably to a proportion that is at least about 90% by mass.

Further, the aerosol which is produced by the activation of a pMDI will generally have a distribution of droplets that immediately or essentially immediately result in aerosolized particles as a result of the evaporation of the carrier components. The particle sizes produced can range from extremely small to relatively large, and it is generally highly desirable for the pMDI to produce a size distribution tending toward the small particles (e.g., in the range of less from about 1 micron to about 5 micron). This desire exists for two reasons. First, many APIs are most effectively absorbed into the body when delivered to the lungs of the user, and smaller particle sizes in this range are most readily absorbed into the lungs. Second, it is known that larger particle sizes (e.g., greater than 5 micron) tend to be deposited to a greater extent along the pathway leading to the lungs, such as in the upper airways (oropharynx and larynx) and the central airways (trachea and primary and secondary bronchus). Thus, for pMDI systems which have a particle distribution tending toward the large particle size, the percentage of effective API deliver is relatively small since a larger proportion of the API is not delivered to the peripheral airways where it is most effective, i.e., tertiary bronchi, terminal bronchioles and alveoli.

WO 2023/039104 discloses a comparison of ipratropium bromide monohydrate formulations and/or anhydrous ipratropium bromide in three propellants, namely, HFC-134a or HFC-152a or HFO-1234ze(E), in combination with ethanol as a cosolvent. This document states that the saturated solubility of ipratropium bromide was significantly lower in HFO-1234ze(E) compositions with 15% ethanol when compared to similar solutions in HFA-134a and HFA-152a.

WO2023/039103 mentions that HFOs have been proposed as propellants for MDIs but also notes that no MDI product has been successfully developed or commercialized using HFOs as a propellant. The '103 publication discloses an MDI that uses a formulation comprising greater than 70% by weight of HFO-1234ze(E), ethanol and at least one active pharmaceutical ingredient (API). The amounts of ethanol disclosed as being used in the '103 formulations range from as low as 0.1 wt. % to as high as 20%.

It is not generally predictable how a change in propellant affects the mass proportion of the formulation delivered to mouth of the user or the particle size distribution of the medication delivered from pMDI, but it is nevertheless highly desirable to provide pMDIs that use low GWP propellant, especially instead of HFC-134a. For this reason, it has been a significant technical challenge to deliver even the same effective mass of medication per actuation achieved using HFC-134a as the propellant.

Applicants have developed pMDIs for APIs in solution that provide not only a low GWP alternative to HFC-134a in pMDIs but which also are unexpectedly able to dramatically increase the effective dose per actuation, as described in detail hereinafter.

### SUMMARY

The present invention includes methods for delivering to the mouth a user a dose of a pharmaceutical formulation at a high percentage of the targeted dose of the pharmaceutical formulation comprising:
a. providing a pMDI comprising:
   i. a canister;
   ii. a pharmaceutical formulation in the canister and comprising: (1) from about 75% by weight to about 95% by weight of HFO-1234ze(E); (2) from about 5% by weight to about 25% by weight of ethanol; and (3) an API in solution in one or more of said HFO-1234ze(E) and said ethanol; and
   iii. a metering valve releasably closing the canister;
   iv. an actuator fluidly connected to the metering valve and having an orifice diameter of about 0.6 mm or less; and
b. actuating said pMDI to introduce into the mouth of the user an aerosol spray formed from said pharmaceutical formulation and containing said API, wherein the amount of said aerosol spray introduced into the mouth of the user is greater than about 80% of said targeted dose.

For the purposes of convenience, methods according to this paragraph are referred to as Pharmaceutical Delivery Method 1A.

The present invention includes methods for delivering to the mouth a user a dose of a pharmaceutical formulation at a high percentage of the targeted dose of the pharmaceutical formulation comprising:
a. providing in a pMDI comprising:
   i. a canister;
   ii. a pharmaceutical formulation in the canister and comprising: (1) from about 75% by weight to about 95% by weight of HFO-1234ze(E); (2) from about 5% by weight to about 25% by weight of ethanol; and (3) an API in solution in one or more of said HFO-1234ze(E) and said ethanol; and
   iii. a metering valve releasably closing the canister;
   iv. an actuator fluidly connected to the metering valve and having an orifice diameter of about 0.5 mm or less; and
b. actuating said pMDI to introduce into the mouth of the user an aerosol spray formed from said pharmaceutical formulation and containing said API, wherein the amount of said aerosol spray introduced into the mouth of the user is at least about 85% of said targeted dose.

For the purposes of convenience, methods according to this paragraph are referred to as Pharmaceutical Delivery Method 1B.

The present invention includes methods for delivering to the mouth a user a dose of a pharmaceutical formulation at a high percentage of the targeted dose of the pharmaceutical formulation comprising:
a. providing in a pMDI comprising:
   i. a canister;
   ii. a pharmaceutical formulation in the canister and comprising: (1) from about 75% by weight to about 95% by weight of HFO-1234ze(E); (2) from about 5% by weight to about 25% by weight of ethanol; and (3) an API in solution in one or more of said HFO-1234ze(E) and said ethanol; and
   iii. a metering valve releasably closing the canister;
   iv. an actuator fluidly connected to the metering valve and having an orifice diameter of about 0.5 mm or less; and
b. actuating said pMDI to introduce into the mouth of the user an aerosol spray formed from said pharmaceutical formulation and containing said API, wherein the amount of said aerosol spray introduced into the mouth of the user is at least about 90% of said targeted dose.

For the purposes of convenience, methods according to this paragraph are referred to as Pharmaceutical Delivery Method 1C.

The present invention includes methods for delivering to the mouth a user a dose of a pharmaceutical formulation at a high percentage of the targeted dose of the pharmaceutical formulation comprising:
a. providing in a pMDI comprising:
   i. a canister;
   ii. a pharmaceutical formulation in the canister and comprising: (1) from about 75% by weight to about 95% by weight of HFO-1234ze(E); (2) from about 5% by weight to about 25% by weight of ethanol; and (3) an API in solution in one or more of said HFO-1234ze(E) and said ethanol; and
   iii. a metering valve releasably closing the canister;
   iv. an actuator fluidly connected to the metering valve and having an orifice diameter of about 0.5 mm or less; and
b. actuating said pMDI to introduce into the mouth of the user an aerosol spray formed from said pharmaceutical formulation and containing said API, wherein the amount of said aerosol spray introduced into the mouth of the user is at least about 95% of said targeted dose.

For the purposes of convenience, methods according to this paragraph are referred to as Pharmaceutical Delivery Method 1D.

The present invention includes methods for delivering to the mouth a user a dose of a pharmaceutical formulation with a high fine particle fraction:
a. providing a pMDI comprising:
   i. a canister;
   ii. a pharmaceutical formulation in the canister and comprising: (1) from about 75% by weight to about 95% by weight of HFO-1234ze(E); (2) from about 5% by weight to about 25% by weight of ethanol; and (3) an API in solution in one or more of said HFO-1234ze(E) and said ethanol; and
   iii. a metering valve releasably closing the canister;
   iv. an actuator fluidly connected to the metering valve and having an orifice diameter that is less than about 0.6 mm; and
b. actuating said pMDI to introduce into the mouth of the user an aerosol spray formed from said pharmaceutical formulation and containing said API, wherein the fine particle fraction of said aerosol spray introduced into the mouth of the user is greater than about 35%.

For the purposes of convenience, methods according to this paragraph are referred to as Pharmaceutical Delivery Method 1E.

The present invention includes methods for delivering to the mouth a user a dose of a pharmaceutical formulation with a high fine particle fraction:
a. providing a pMDI comprising:
   i. a canister;
   ii. a pharmaceutical formulation in the canister and comprising: (1) from about 75% by weight to about 95% by weight of HFO-1234ze(E); (2) from about 5% by weight to about 25% by weight of ethanol; and (3) an API in solution in one or more of said HFO-1234ze(E) and said ethanol; and
   iii. a metering valve releasably closing the canister;
   iv. an actuator fluidly connected to the metering valve and having an orifice diameter that is less than about 0.6 mm; and
b. actuating said pMDI to introduce into the mouth of the user an aerosol spray formed from said pharmaceutical formulation and containing said API, wherein the fine particle fraction of said aerosol spray introduced into the mouth of the user is greater than about 45%.

For the purposes of convenience, methods according to this paragraph are referred to as Pharmaceutical Delivery Method 1F.

The present invention includes methods for delivering to the mouth a user a dose of a pharmaceutical formulation with a high fine particle fraction:
a. providing a pMDI comprising:
   i. a canister;
   ii. a pharmaceutical formulation in the canister and comprising: (1) from about 75% by weight to about 95% by weight of HFO-1234ze(E); (2) from about 5% by weight to about 25% by weight of ethanol; and (3) an API in solution in one or more of said HFO-1234ze(E) and said ethanol; and
   iii. a metering valve releasably closing the canister;
   iv. an actuator fluidly connected to the metering valve and having an orifice diameter that is less than about 0.6 mm; and
b. actuating said pMDI to introduce into the mouth of the user an aerosol spray formed from said pharmaceutical formulation and containing said API, wherein the fine particle fraction of said aerosol spray introduced into the mouth of the user is greater than about 60%.

For the purposes of convenience, methods according to this paragraph are referred to as Pharmaceutical Delivery Method 1G.

The present invention includes methods for delivering to the mouth a user a dose of a pharmaceutical formulation with a high fine particle fraction:
a. providing a pMDI comprising:
   i. a canister;
   ii. a pharmaceutical formulation in the canister and comprising: (1) from about 75% by weight to about 95% by weight of HFO-1234ze(E); (2) from about 5% by weight to about 25% by weight of ethanol; and (3) an API in solution in one or more of said HFO-1234ze(E) and said ethanol; and
   iii. a metering valve releasably closing the canister;
   iv. an actuator fluidly connected to the metering valve and having an orifice diameter that is less than about 0.6 mm; and
b. actuating said pMDI to introduce into the mouth of the user an aerosol spray formed from said pharmaceutical formulation and containing said API, wherein the fine particle fraction of said aerosol spray introduced into the mouth of the user is greater than about 70%.

For the purposes of convenience, methods according to this paragraph are referred to as Pharmaceutical Delivery Method 1H.

The present invention includes methods for delivering to the mouth a user a dose of a pharmaceutical formulation with a high fine particle fraction:
a. providing a pMDI comprising:
   i. a canister;
   ii. a pharmaceutical formulation in the canister and comprising: (1) from about 75% by weight to about 95% by weight of HFO-1234ze(E); (2) from about 5% by weight to about 25% by weight of ethanol; and (3) an API in solution in one or more of said HFO-1234ze(E) and said ethanol; and
   iii. a metering valve releasably closing the canister;
   iv. an actuator fluidly connected to the metering valve and having an orifice diameter that is less than about 0.6 mm; and
b. actuating said pMDI to introduce into the mouth of the user an aerosol spray formed from said pharmaceutical formulation and containing said API, wherein the fine particle fraction of said aerosol spray introduced into the mouth of the user is greater than about 80%.

For the purposes of convenience, methods according to this paragraph are referred to as Pharmaceutical Delivery Method 11.

The present invention includes methods for delivering to the mouth a user a dose of a pharmaceutical formulation:
a. providing a pMDI comprising:
   i. a canister;
   ii. a pharmaceutical formulation in the canister and comprising: (1) from about 75% by weight to about 95% by weight of HFO-1234ze(E); (2) from about 5% by weight to about 25% by weight of ethanol; and (3) an API in solution in one or more of said HFO-1234ze(E) and said ethanol; and
   iii. a metering valve releasably closing the canister;
   iv. an actuator fluidly connected to the metering valve and having an orifice diameter that is less than about 0.6 mm; and
b. actuating said pMDI to introduce into the mouth of the user an aerosol spray formed from said pharmaceutical formulation and containing said API, wherein the fine particle fraction of said aerosol spray introduced into the mouth of the user is greater than about 35% and wherein the amount of said aerosol spray introduced into the mouth of the user is greater than about 80% of said targeted dose.

For the purposes of convenience, methods according to this paragraph are referred to as Pharmaceutical Delivery Method 1J.

The present invention includes methods for delivering to the mouth a user a dose of a pharmaceutical formulation at a high percentage of the targeted dose of the pharmaceutical formulation comprising:
a. providing in a pMDI comprising:
   i. a canister;
   ii. a pharmaceutical formulation in the canister and comprising: (1) from about 75% by weight to about 95% by weight of HFO-1234ze(E); (2) from about 5% by weight to about 25% by weight of ethanol; and (3) an API in solution in one or more of said HFO-1234ze(E) and said ethanol, said API selected from the group consisting of ipratropium, beclomethasone, formoterol, glycopyrrolate and combinations of two or more of these; and
   iii. a metering valve releasably closing the canister;
   iv. an actuator fluidly connected to the metering valve and having an orifice diameter of about 0.5 mm or less; and
b. actuating said pMDI to introduce into the mouth of the user an aerosol spray formed from said pharmaceutical formulation and containing said API, wherein the amount of said aerosol spray introduced into the mouth of the user is greater than about 80% of said targeted dose.

For the purposes of convenience, methods according to this paragraph are referred to as Pharmaceutical Delivery Method 2A.

The present invention includes methods for delivering to the mouth a user a dose of a pharmaceutical formulation at a high percentage of the targeted dose of the pharmaceutical formulation comprising:
a. providing in a pMDI comprising:
   i. a canister;
   ii. a pharmaceutical formulation in the canister and comprising: (1) from about 75% by weight to about 95% by weight of HFO-1234ze(E); (2) from about 5% by weight to about 25% by weight of ethanol; and (3) an API in solution in one or more of said HFO-1234ze(E) and said ethanol, said API selected from the group consisting of ipratropium, beclomethasone, formoterol, glycopyrrolate and combinations of two or more of these; and
   iii. a metering valve releasably closing the canister;
   iv. an actuator fluidly connected to the metering valve and having an orifice diameter of about 0.5 mm or less; and
b. actuating said pMDI to introduce into the mouth of the user an aerosol spray formed from said pharmaceutical formulation and containing said API, wherein the amount of said aerosol spray introduced into the mouth of the user is at least about 85% of said targeted dose.

For the purposes of convenience, methods according to this paragraph are referred to as Pharmaceutical Delivery Method 2B.

The present invention includes methods for delivering to the mouth a user a dose of a pharmaceutical formulation at a high percentage of the targeted dose of the pharmaceutical formulation comprising:
a. providing in a pMDI comprising:
   i. a canister;
   ii. a pharmaceutical formulation in the canister and comprising: (1) from about 75% by weight to about 95% by weight of HFO-1234ze(E); (2) from about 5% by weight to about 25% by weight of ethanol; and (3) an API in solution in one or more of said HFO-1234ze(E) and said ethanol, said API selected from the group consisting of ipratropium, beclomethasone, formoterol, glycopyrrolate and combinations of two or more of these; and
   iii. a metering valve releasably closing the canister;
   iv. an actuator fluidly connected to the metering valve and having an orifice diameter of about 0.5 mm or less; and
b. actuating said pMDI to introduce into the mouth of the user an aerosol spray formed from said pharmaceutical formulation and containing said API, wherein the amount of said aerosol spray introduced into the mouth of the user is at least about 90% of said targeted dose.

For the purposes of convenience, methods according to this paragraph are referred to as Pharmaceutical Delivery Method 2C.

The present invention includes methods for delivering to the mouth a user a dose of a pharmaceutical formulation at a high percentage of the targeted dose of the pharmaceutical formulation comprising:
a. providing in a pMDI comprising:
   i. a canister;
   ii. a pharmaceutical formulation in the canister and comprising: (1) from about 75% by weight to about 95% by weight of HFO-1234ze(E); (2) from about 5% by weight to about 25% by weight of ethanol; and (3) an API in solution in one or more of said HFO-1234ze(E) and said ethanol, said API selected from the group consisting of ipratropium, beclomethasone, formoterol, glycopyrrolate and combinations of two or more of these; and
   iii. a metering valve releasably closing the canister;
   iv. an actuator fluidly connected to the metering valve and having an orifice diameter of about 0.5 mm or less; and
b. actuating said pMDI to introduce into the mouth of the user an aerosol spray formed from said pharmaceutical formulation and containing said API, wherein the amount of said aerosol spray introduced into the mouth of the user is at least about 95% of said targeted dose.

For the purposes of convenience, methods according to this paragraph are referred to as Pharmaceutical Delivery Method 2D.

The present invention includes methods for delivering to the mouth a user a dose of a pharmaceutical formulation at a high percentage of the targeted dose of the pharmaceutical formulation comprising:
a. providing in a pMDI comprising:
   i. a canister;
   ii. a pharmaceutical formulation in the canister and comprising: (1) from about 75% by weight to about 95% by weight of HFO-1234ze(E); (2) from about 5% by weight to about 25% by weight of ethanol; and (3) an API in solution in one or more of said HFO-1234ze(E) and said ethanol, said API selected from the group consisting of ipratropium, beclomethasone, formoterol, glycopyrrolate and combinations of two or more of these; and
   iii. a metering valve releasably closing the canister;
   iv. an actuator fluidly connected to the metering valve and having an orifice diameter of about 0.5 mm or less; and
b. actuating said pMDI to introduce into the mouth of the user an aerosol spray formed from said pharmaceutical formulation and containing said API, wherein the amount of said aerosol spray introduced into the mouth of the user is at least about 100% of said targeted dose.

For the purposes of convenience, methods according to this paragraph are referred to as Pharmaceutical Delivery Method 2E.

The present invention includes methods for delivering to the mouth a user a dose of a pharmaceutical formulation at a high percentage of the targeted dose of the pharmaceutical formulation comprising:
a. providing in a pMDI comprising:
   i. a canister;
   ii. a pharmaceutical formulation in the canister and comprising: (1) from about 75% by weight to about 95% by weight of HFO-1234ze(E); (2) from about 5% by weight to about 25% by weight of ethanol; and (3) an API in solution in one or more of said HFO-1234ze(E) and said ethanol, said API selected from the group consisting of ipratropium, beclomethasone, formoterol, glycopyrrolate and combinations of two or more of these; and
   iii. a metering valve releasably closing the canister;
   iv. an actuator fluidly connected to the metering valve and having an orifice diameter of about 0.5 mm or less; and
b. actuating said pMDI to introduce into the mouth of the user an aerosol spray formed from said pharmaceutical formulation and containing said API, wherein the amount of said aerosol spray introduced into the mouth of the user is at least about 110% of the amount of said aerosol spray introduced into the mouth of the user when HFC-134a is used in place of said HFC-1234ze(E) on a weight for weight basis in said formulation.

For the purposes of convenience, methods according to this paragraph are referred to as Pharmaceutical Delivery Method 2F.

The present invention includes methods for delivering to the mouth a user a dose of a pharmaceutical formulation at a high percentage of the targeted dose of the pharmaceutical formulation comprising:
a. providing in a pMDI comprising:
   i. a canister;
   ii. a pharmaceutical formulation in the canister and comprising: (1) from about 75% by weight to about 95% by weight of HFO-1234ze(E); (2) from about 5% by weight to about 25% by weight of ethanol; and (3) an API in solution in one or more of said HFO-1234ze(E) and said ethanol, said API selected from the group consisting of ipratropium, beclomethasone, formoterol, glycopyrrolate and combinations of two or more of these; and
   iii. a metering valve releasably closing the canister;
   iv. an actuator fluidly connected to the metering valve and having an orifice diameter of about 0.5 mm or less; and
b. actuating said pMDI to introduce into the mouth of the user an aerosol spray formed from said pharmaceutical formulation and containing said API, wherein the amount of said aerosol spray introduced into the mouth of the user is at least about 120% of the amount of said aerosol spray introduced into the mouth of the user when HFC-134a is used in place of said HFC-1234ze(E) on a weight for weight basis in said formulation.

For the purposes of convenience, methods according to this paragraph are referred to as Pharmaceutical Delivery Method 2G.

The present invention includes methods for delivering to the mouth a user a dose of a pharmaceutical formulation at a high percentage of the targeted dose of the pharmaceutical formulation comprising:
a. providing in a pMDI comprising:
   i. a canister;
   ii. a pharmaceutical formulation in the canister and comprising: (1) from about 75% by weight to about 95% by weight of HFO-1234ze(E); (2) from about 5% by weight to about 25% by weight of ethanol; and (3) an API in solution in one or more of said HFO-1234ze(E) and said ethanol, said API selected from the group consisting of ipratropium, beclomethasone, formoterol, glycopyrrolate and combinations of two or more of these; and
   iii. a metering valve releasably closing the canister;
   iv. an actuator fluidly connected to the metering valve and having an orifice diameter of about 0.5 mm or less; and
b. actuating said pMDI to introduce into the mouth of the user an aerosol spray formed from said pharmaceutical formulation and containing said API, wherein the amount of said aerosol spray introduced into the mouth of the user is at least about 130% of the amount of said aerosol spray introduced into the mouth of the user when HFC-134a is used in place of said HFC-1234ze(E) on a weight for weight basis in said formulation.

For the purposes of convenience, methods according to this paragraph are referred to as Pharmaceutical Delivery Method 2H.

The present invention includes methods for delivering to the mouth a user a dose of a pharmaceutical formulation comprising:
a. providing in a pMDI comprising:
   i. a canister;
   ii. a pharmaceutical formulation in the canister and comprising: (1) from about 75% by weight to about 95% by weight of HFO-1234ze(E); (2) from about 5% by weight to about 25% by weight of ethanol; and (3) an API in solution in one or more of said HFO-1234ze(E) and said ethanol, said API selected from the group consisting of ipratropium, beclomethasone, formoterol, glycopyrrolate and combinations of two or more of these; and
   iii. a metering valve releasably closing the canister;
   iv. an actuator fluidly connected to the metering valve and having an orifice diameter of about 0.5 mm or less; and
b. actuating said pMDI to introduce into the mouth of the user an aerosol spray formed from said pharmaceutical formulation and containing said API, wherein the fine particle fraction of said aerosol spray introduced into the mouth of the user is greater than about 35%.

For the purposes of convenience, methods according to this paragraph are referred to as Pharmaceutical Delivery Method 2I.

The present invention includes methods for delivering to the mouth a user a dose of a pharmaceutical formulation comprising:
a. providing in a pMDI comprising:
   i. a canister;
   ii. a pharmaceutical formulation in the canister and comprising: (1) from about 75% by weight to about 95% by weight of HFO-1234ze(E); (2) from about 5% by weight to about 25% by weight of ethanol; and (3) an API in solution in one or more of said HFO-1234ze(E) and said ethanol, said API selected from the group consisting of ipratropium, beclomethasone, formoterol, glycopyrrolate and combinations of two or more of these; and
   iii. a metering valve releasably closing the canister;
   iv. an actuator fluidly connected to the metering valve and having an orifice diameter of about 0.5 mm or less; and
b. actuating said pMDI to introduce into the mouth of the user an aerosol spray formed from said pharmaceutical formulation and containing said API, wherein the fine particle fraction of said aerosol spray introduced into the mouth of the user is greater than about 45%.

For the purposes of convenience, methods according to this paragraph are referred to as Pharmaceutical Delivery Method 2J.

The present invention includes methods for delivering to the mouth a user a dose of a pharmaceutical formulation comprising:
a. providing in a pMDI comprising:
   i. a canister;
   ii. a pharmaceutical formulation in the canister and comprising: (1) from about 75% by weight to about 95% by weight of HFO-1234ze(E); (2) from about 5% by weight to about 25% by weight of ethanol; and (3) an API in solution in one or more of said HFO-1234ze(E) and said ethanol, said API selected from the group consisting of ipratropium, beclomethasone, formoterol, glycopyrrolate and combinations of two or more of these; and
   iii. a metering valve releasably closing the canister;
   iv. an actuator fluidly connected to the metering valve and having an orifice diameter of about 0.5 mm or less; and
b. actuating said pMDI to introduce into the mouth of the user an aerosol spray formed from said pharmaceutical formulation and containing said API, wherein the fine particle fraction of said aerosol spray introduced into the mouth of the user is greater than about 60%.

For the purposes of convenience, methods according to this paragraph are referred to as Pharmaceutical Delivery Method 2K.

The present invention includes methods for delivering to the mouth a user a dose of a pharmaceutical formulation comprising:
a. providing in a pMDI comprising:
   i. a canister;
   ii. a pharmaceutical formulation in the canister and comprising: (1) from about 75% by weight to about 95% by weight of HFO-1234ze(E); (2) from about 5% by weight to about 25% by weight of ethanol; and (3) an API in solution in one or more of said HFO-1234ze(E) and said ethanol, said API selected from the group consisting of ipratropium, beclomethasone, formoterol, glycopyrrolate and combinations of two or more of these; and
   iii. a metering valve releasably closing the canister;
   iv. an actuator fluidly connected to the metering valve and having an orifice diameter of about 0.5 mm or less; and
b. actuating said pMDI to introduce into the mouth of the user an aerosol spray formed from said pharmaceutical formulation and containing said API, wherein the fine particle fraction of said aerosol spray introduced into the mouth of the user is greater than about 80%.

For the purposes of convenience, methods according to this paragraph are referred to as Pharmaceutical Delivery Method 2L.

The present invention includes methods for delivering to the mouth a user a dose of a pharmaceutical formulation at a high percentage of the targeted dose of the pharmaceutical formulation comprising:
a. providing in a pMDI comprising:
   i. a canister;
   ii. a pharmaceutical formulation in the canister and comprising: (1) from about 75% by weight to about 95% by weight of HFO-1234ze(E); (2) from about 5% by weight to about 25% by weight of ethanol; and (3) an API in solution in one or more of said HFO-1234ze(E) and said ethanol, said API selected from the group consisting of ipratropium, beclomethasone, formoterol, glycopyrrolate and combinations of two or more of these; and
   iii. a metering valve releasably closing the canister;
   iv. an actuator fluidly connected to the metering valve and having an orifice diameter of about 0.5 mm or less; and
b. actuating said pMDI to introduce into the mouth of the user an aerosol spray formed from said pharmaceutical formulation and containing said API, wherein the fine particle fraction of said aerosol spray introduced into the mouth of the user is greater than about 35% and wherein the amount of said aerosol spray introduced into the mouth of the user is at least about 90% of said targeted dose.

For the purposes of convenience, methods according to this paragraph are referred to as Pharmaceutical Delivery Method 2M.

The present invention includes methods for delivering a dose of ipratropium bromide comprising:
a. providing in a pMDI comprising:
   i. a canister;
   ii. a formulation comprising ipratropium bromide in solution contained in the canister, said formulation comprising: (1) from about 75% by weight to about 95% by weight of HFO-1234ze(E); (2) from about 5% by weight to about 25% by weight of ethanol; and (3) ipratropium bromide; and
   iii. a metering valve releasably closing the canister and fluidly connected to an actuator having an orifice diameter of about 0.5 mm or less; and
b. actuating said pMDI to produce an aerosol spray comprising said ipratropium bromide, wherein the amount of said aerosol spray introduced into the mouth of the user is at greater than about 80% of said targeted dose.

For the purposes of convenience, methods according to this paragraph are referred to as Pharmaceutical Delivery Method 3A1.

The present invention includes methods for delivering a dose of ipratropium bromide comprising:
a. providing in a pMDI comprising:
   i. a canister;
   ii. a formulation comprising ipratropium bromide in solution contained in the canister, said formulation comprising: (1) from about 75% by weight to about 95% by weight of HFO-1234ze(E); (2) from about 5% by weight to about 25% by weight of ethanol; and (3) ipratropium bromide; and
   iii. a metering valve releasably closing the canister and fluidly connected to an actuator having an orifice diameter of about 0.5 mm or less; and
b. actuating said pMDI to produce an aerosol spray comprising said ipratropium bromide, wherein the amount of said aerosol spray introduced into the mouth of the user is at greater than about 80% of said targeted dose and wherein said wherein the fine particle fraction of said aerosol spray introduced into the mouth of the user is greater than about 35%.

For the purposes of convenience, methods according to this paragraph are referred to as Pharmaceutical Delivery Method 3A2.

The present invention includes methods for delivering a dose of ipratropium bromide comprising:
a. providing in a pMDI comprising:
   i. a canister;
   ii. a formulation comprising ipratropium bromide in solution contained in the canister, said formulation comprising: (1) from about 75% by weight to about 95% by weight of HFO-1234ze(E); (2) from about 5% by weight to about 25% by weight of ethanol; and (3) ipratropium bromide; and
   iii. a metering valve releasably closing the canister and fluidly connected to an actuator having an orifice diameter of about 0.5 mm or less; and
b. actuating said pMDI to produce an aerosol spray comprising said ipratropium bromide, wherein the amount of said aerosol spray introduced into the mouth of the user is at least about 85% of said targeted dose.

For the purposes of convenience, methods according to this paragraph are referred to as Pharmaceutical Delivery Method 3B.

The present invention includes methods for delivering a dose of ipratropium bromide comprising:
a. providing in a pMDI comprising:
   i. a canister;
   ii. a formulation comprising ipratropium bromide in solution contained in the canister, said formulation comprising: (1) from about 75% by weight to about 95% by weight of HFO-1234ze(E); (2) from about 5% by weight to about 25% by weight of ethanol; and (3) ipratropium bromide; and
   iii. a metering valve releasably closing the canister and fluidly connected to an actuator having an orifice diameter of about 0.5 mm or less; and
b. actuating said pMDI to produce an aerosol spray comprising said ipratropium bromide, wherein the amount of said aerosol spray introduced into the mouth of the user is at least about 90% of said targeted dose.

For the purposes of convenience, methods according to this paragraph are referred to as Pharmaceutical Delivery Method 3C.

The present invention includes methods for delivering a dose of ipratropium bromide comprising:
a. providing in a pMDI comprising:
   i. a canister;
   ii. a formulation comprising ipratropium bromide in solution contained in the canister, said formulation comprising: (1) from about 75% by weight to about 95% by weight of HFO-1234ze(E); (2) from about 5% by weight to about 25% by weight of ethanol; and (3) ipratropium bromide; and
   iii. a metering valve releasably closing the canister and fluidly connected to an actuator having an orifice diameter of about 0.5 mm or less; and
b. actuating said pMDI to produce an aerosol spray comprising said ipratropium bromide, wherein the amount of said aerosol spray introduced into the mouth of the user is at least about 95% of said targeted dose.

For the purposes of convenience, methods according to this paragraph are referred to as Pharmaceutical Delivery Method 3D.

The present invention includes methods for delivering a dose of ipratropium bromide comprising:
a. providing in a pMDI comprising:
   i. a canister;
   ii. a formulation comprising ipratropium bromide in solution contained in the canister, said formulation comprising: (1) from about 75% by weight to about 95% by weight of HFO-1234ze(E); (2) from about 5% by weight to about 25% by weight of ethanol; and (3) ipratropium bromide; and
   iii. a metering valve releasably closing the canister and fluidly connected to an actuator having an orifice diameter of about 0.5 mm or less; and
b. actuating said pMDI to produce an aerosol spray comprising said ipratropium bromide, wherein the amount of said aerosol spray introduced into the mouth of the user is at least about 110% of the amount of said aerosol spray introduced into the mouth of the user when HFC-134a is used in place of said HFC-1234ze(E) on a weight for weight basis in said formulation.

For the purposes of convenience, methods according to this paragraph are referred to as Pharmaceutical Delivery Method 3E.

The present invention includes methods for delivering a dose of ipratropium bromide comprising:
a. providing in a pMDI comprising:
   i. a canister;
   ii. a formulation comprising ipratropium bromide in solution contained in the canister, said formulation comprising: (1) from about 75% by weight to about 95% by weight of HFO-1234ze(E); (2) from about 5% by weight to about 25% by weight of ethanol; and (3) ipratropium bromide; and
b. actuating said pMDI to produce an aerosol spray comprising said ipratropium bromide, wherein the amount of said aerosol spray introduced into the mouth of the user is at least about 120% of the amount of said aerosol spray introduced into the mouth of the user when HFC-134a is used in place of said HFC-1234ze(E) on a weight for weight basis in said formulation.

For the purposes of convenience, methods according to this paragraph are referred to as Pharmaceutical Delivery Method 3F.

The present invention includes methods for delivering a dose of ipratropium bromide comprising:
a. providing in a pMDI comprising:
   i. a canister;
   ii. a formulation comprising ipratropium bromide in solution contained in the canister, said formulation comprising: (1) from about 75% by weight to about 95% by weight of HFO-1234ze(E); (2) from about 5% by weight to about 25% by weight of ethanol; and (3) ipratropium bromide; and
   iii. a metering valve releasably closing the canister and fluidly connected to an actuator having an orifice diameter of about 0.5 mm or less; and
b. actuating said pMDI to produce an aerosol spray comprising said ipratropium bromide, wherein the amount of said aerosol spray introduced into the mouth of the user is at least about 130% of the amount of said aerosol spray introduced into the mouth of the user when HFC-134a is used in place of said HFC-1234ze(E) on a weight for weight basis in said formulation.

For the purposes of convenience, methods according to this paragraph are referred to as Pharmaceutical Delivery Method 3G.

The present invention includes methods for delivering a dose of formoterol comprising:
a. providing in a pMDI comprising:
   i. a canister;
   ii. a formulation comprising formoterol in solution contained in the canister, said formulation comprising: (1) from about 80% by weight to about 90% by weight of HFO-1234ze(E); (2) from about 10% by weight to about 20% by weight of ethanol; and (3) formoterol; and
   iii. a metering valve releasably closing the canister and fluidly connected to an actuator having an orifice diameter of about 0.5 mm or less; and
b. actuating said pMDI to produce an aerosol spray comprising said formoterol, wherein the amount of said aerosol spray introduced into the mouth of the user is at greater than about 80% of said targeted dose.

For the purposes of convenience, methods according to this paragraph are referred to as Pharmaceutical Delivery Method 4A1.

The present invention includes methods for delivering a dose of formoterol comprising:
a. providing in a pMDI comprising:
   i. a canister;
   ii. a formulation comprising formoterol in solution contained in the canister, said formulation comprising: (1) from about 80% by weight to about 90% by weight of HFO-1234ze(E); (2) from about 10% by weight to about 20% by weight of ethanol; and (3) formoterol; and
   iii. a metering valve releasably closing the canister and fluidly connected to an actuator having an orifice diameter of about
b. actuating said pMDI to produce an aerosol spray comprising said formoterol, wherein the amount of said aerosol spray introduced into the mouth of the user is at greater than about 80% of said targeted dose and wherein the fine particle fraction of said aerosol spray introduced into the mouth of the user is greater than about 25%.

For the purposes of convenience, methods according to this paragraph are referred to as Pharmaceutical Delivery Method 4A2.

The present invention includes methods for delivering a dose of formoterol comprising:
a. providing in a pMDI comprising:
   i. a canister;
   ii. a formulation comprising formoterol in solution contained in the canister, said formulation comprising: (1) from about 80% by weight to about 90% by weight of HFO-1234ze(E); (2) from about 10% by weight to about 20% by weight of ethanol; and (3) formoterol; and
   iii. a metering valve releasably closing the canister and fluidly connected to an actuator having an orifice diameter of about 0.5 mm or less; and
b. actuating said pMDI to produce an aerosol spray comprising said formoterol, wherein the amount of said aerosol spray introduced into the mouth of the user is at greater than about 80% of said targeted dose and wherein the fine particle fraction of said aerosol spray introduced into the mouth of the user is greater than about 35%.

For the purposes of convenience, methods according to this paragraph are referred to as Pharmaceutical Delivery Method 4A2.

The present invention includes methods for delivering a dose of formoterol comprising:
a. providing in a pMDI comprising:
   i. a canister;
   ii. a formulation comprising formoterol in solution contained in the canister, said formulation comprising: (1) from about 80% by weight to about 90% by weight of HFO-1234ze(E); (2) from about 10% by weight to about 20% by weight of ethanol; and (3) formoterol; and
   iii. a metering valve releasably closing the canister and fluidly connected to an actuator having an orifice diameter of less than 0.4 mm; and
b. actuating said pMDI to produce an aerosol spray comprising said formoterol, wherein the amount of said aerosol spray introduced into the mouth of the user is at greater than about 80% of said targeted dose and wherein the fine particle fraction of said aerosol spray introduced into the mouth of the user is greater than about 35%.

For the purposes of convenience, methods according to this paragraph are referred to as Pharmaceutical Delivery Method 4A3.

The present invention includes methods for delivering a dose of formoterol comprising:
a. providing in a pMDI comprising:
   i. a canister;
   ii. a formulation comprising formoterol in solution contained in the canister, said formulation comprising: (1) from about 80% by weight to about 90% by weight of HFO-1234ze(E); (2) from about 10% by weight to about 20% by weight of ethanol; and (3) formoterol; and
   iii. a metering valve releasably closing the canister and fluidly connected to an actuator having an orifice diameter of less than 0.3 mm; and
b. actuating said pMDI to produce an aerosol spray comprising said formoterol, wherein the amount of said aerosol spray introduced into the mouth of the user is at greater than about 80% of said targeted dose and wherein the fine particle fraction of said aerosol spray introduced into the mouth of the user is greater than about 35%.

For the purposes of convenience, methods according to this paragraph are referred to as Pharmaceutical Delivery Method 4A4.

The present invention includes methods for delivering a dose of formoterol comprising:
a. providing in a pMDI comprising:
   i. a canister;
   ii. a formulation comprising formoterol in solution contained in the canister, said formulation comprising: (1) from about 80% by weight to about 90% by weight of HFO-1234ze(E); (2) from about 10% by weight to about 20% by weight of ethanol; and (3) formoterol; and
   iii. a metering valve releasably closing the canister and fluidly connected to an actuator having an orifice diameter of about 0.5 mm or less; and
b. actuating said pMDI to produce an aerosol spray comprising said formoterol, wherein the amount of said aerosol spray introduced into the mouth of the user is at least about 85% of said targeted dose.

For the purposes of convenience, methods according to this paragraph are referred to as Pharmaceutical Delivery Method 4B.

The present invention includes methods for delivering a dose of formoterol comprising:
a. providing in a pMDI comprising:
   i. a canister;
   ii. a formulation comprising formoterol in solution contained in the canister, said formulation comprising: (1) from about 80% by weight to about 90% by weight of HFO-1234ze(E); (2) from about 10% by weight to about 20% by weight of ethanol; and (3) formoterol; and
   iii. a metering valve releasably closing the canister and fluidly connected to an actuator having an orifice diameter of about 0.5 mm or less; and
b. actuating said pMDI to produce an aerosol spray comprising said formoterol, wherein the amount of said aerosol spray introduced into the mouth of the user is at least about 90% of said targeted dose.

For the purposes of convenience, methods according to this paragraph are referred to as Pharmaceutical Delivery Method 4C.

The present invention includes methods for delivering a dose of formoterol comprising:
a. providing in a pMDI comprising:
   i. a canister;
   ii. a formulation comprising formoterol in solution contained in the canister, said formulation comprising: (1) from about 80% by weight to about 90% by weight of HFO-1234ze(E); (2) from about 10% by weight to about 20% by weight of ethanol; and (3) formoterol; and
   iii. a metering valve releasably closing the canister and fluidly connected to an actuator having an orifice diameter of about 0.5 mm or less; and
b. actuating said pMDI to produce an aerosol spray comprising said formoterol, wherein the amount of said aerosol spray introduced into the mouth of the user is at least about 95% of said targeted dose.

For the purposes of convenience, methods according to this paragraph are referred to as Pharmaceutical Delivery Method 4D.

The present invention includes methods for delivering a dose of glycopyrrolate comprising:
a. providing in a pMDI comprising:
   i. a canister;
   ii. a formulation comprising glycopyrrolate in solution contained in the canister, said formulation comprising: (1) from about 75% by weight to about 90% by weight of HFO-1234ze(E); (2) from about 10% by weight to about 25% by weight of ethanol; and (3) glycopyrrolate; and
   iii. a metering valve releasably closing the canister and fluidly connected to an actuator having an orifice diameter of about 0.5 mm or less; and
b. actuating said pMDI to produce an aerosol spray comprising said glycopyrrolate, wherein the amount of said aerosol spray introduced into the mouth of the user is at greater than about 80% of said targeted dose.

For the purposes of convenience, methods according to this paragraph are referred to as Pharmaceutical Delivery Method 5A1.

The present invention includes methods for delivering a dose of glycopyrrolate comprising:
a. providing in a pMDI comprising:
   i. a canister;
   ii. a formulation comprising glycopyrrolate in solution contained in the canister, said formulation comprising: (1) from about 75% by weight to about 90% by weight of HFO-1234ze(E); (2) from about 10% by weight to about 25% by weight of ethanol; and (3) glycopyrrolate; and
   iii. a metering valve releasably closing the canister and fluidly connected to an actuator having an orifice diameter of about 0.5 mm or less; and
b. actuating said pMDI to produce an aerosol spray comprising said glycopyrrolate, wherein the amount of said aerosol spray introduced into the mouth of the user is at greater than about 80% of said targeted dose and wherein the fine particle fraction of said aerosol spray introduced into the mouth of the user is greater than about 35%.

For the purposes of convenience, methods according to this paragraph are referred to as Pharmaceutical Delivery Method 5A2.

The present invention includes methods for delivering a dose of glycopyrrolate comprising:
a. providing in a pMDI comprising:
   i. a canister;
   ii. a formulation comprising glycopyrrolate in solution contained in the canister, said formulation comprising: (1) from about 75% by weight to about 90% by weight of HFO-1234ze(E); (2) from about 10% by weight to about 25% by weight of ethanol; and (3) glycopyrrolate; and
   iii. a metering valve releasably closing the canister and fluidly connected to an actuator having an orifice diameter of about 0.5 mm or less; and
b. actuating said pMDI to produce an aerosol spray comprising said glycopyrrolate, wherein the amount of said aerosol spray introduced into the mouth of the user is at greater than about 80% of said targeted dose and wherein the fine particle fraction of said aerosol spray introduced into the mouth of the user is greater than about 40%.

For the purposes of convenience, methods according to this paragraph are referred to as Pharmaceutical Delivery Method 5A3.

The present invention includes methods for delivering a dose of glycopyrrolate comprising:
a. providing in a pMDI comprising:
   i. a canister;
   ii. a formulation comprising glycopyrrolate in solution contained in the canister, said formulation comprising: (1) from about 75% by weight to about 90% by weight of HFO-1234ze(E); (2) from about 10% by weight to about 25% by weight of ethanol; and (3) glycopyrrolate; and
   iii. a metering valve releasably closing the canister and fluidly connected to an actuator having an orifice diameter of less than 0.3 mm; and
b. actuating said pMDI to produce an aerosol spray comprising said glycopyrrolate, wherein the amount of said aerosol spray introduced into the mouth of the user is at greater than about 80% of said targeted dose and wherein the fine particle fraction of said aerosol spray introduced into the mouth of the user is greater than about 40%.

For the purposes of convenience, methods according to this paragraph are referred to as Pharmaceutical Delivery Method 5A4.

The present invention includes methods for delivering a dose of glycopyrrolate comprising:
a. providing in a pMDI comprising:
   i. a canister;
   ii. a formulation comprising glycopyrrolate in solution contained in the canister, said formulation comprising: (1) from about 75% by weight to about 90% by weight of HFO-1234ze(E); (2) from about 10% by weight to about 25% by weight of ethanol; and (3) glycopyrrolate; and
   iii. a metering valve releasably closing the canister and fluidly connected to an actuator having an orifice diameter of about 0.5 mm or less; and
b. actuating said pMDI to produce an aerosol spray comprising said glycopyrrolate, wherein the amount of said aerosol spray introduced into the mouth of the user is at least about 85% of said targeted dose.

For the purposes of convenience, methods according to this paragraph are referred to as Pharmaceutical Delivery Method 5B.

The present invention includes methods for delivering a dose of glycopyrrolate comprising:
a. providing in a pMDI comprising:
   i. a canister;
   ii. a formulation comprising glycopyrrolate in solution contained in the canister, said formulation comprising: (1) from about 75% by weight to about 90% by weight of HFO-1234ze(E); (2) from about 10% by weight to about 25% by weight of ethanol; and (3) glycopyrrolate; and
   iii. a metering valve releasably closing the canister and fluidly connected to an actuator having an orifice diameter of about 0.5 mm or less; and
b. actuating said pMDI to produce an aerosol spray comprising said glycopyrrolate, wherein the amount of said aerosol spray introduced into the mouth of the user is at least about 90% of said targeted dose.

For the purposes of convenience, methods according to this paragraph are referred to as Pharmaceutical Delivery Method 5C.

The present invention includes methods for delivering a dose of glycopyrrolate comprising:
a. providing in a pMDI comprising:
   i. a canister;
   ii. a formulation comprising glycopyrrolate in solution contained in the canister, said formulation comprising: (1) from about 75% by weight to about 90% by weight of HFO-1234ze(E); (2) from about 10% by weight to about 25% by weight of ethanol; and (3) glycopyrrolate; and
   iii. a metering valve releasably closing the canister and fluidly connected to an actuator having an orifice diameter of about 0.5 mm or less; and
b. actuating said pMDI to produce an aerosol spray comprising said glycopyrrolate, wherein the amount of said aerosol spray introduced into the mouth of the user is at least about 95% of said targeted dose.

For the purposes of convenience, methods according to this paragraph are referred to as Pharmaceutical Delivery Method 5D.

The present invention includes methods for delivering a dose of beclomethasone comprising:
a. providing in a pMDI comprising:
   i. a canister;
   ii. a formulation comprising ipratropium bromide in solution contained in the canister, said formulation comprising: (1) from about 85% by weight to about 95% by weight of HFO-1234ze(E); (2) from about 5% by weight to about 15% by weight of ethanol; and (3) beclomethasone; and
   iii. a metering valve releasably closing the canister and fluidly connected to an actuator having an orifice diameter of about 0.5 mm or less; and
b. actuating said pMDI to produce an aerosol spray comprising said beclomethasone, wherein the amount of said aerosol spray introduced into the mouth of the user is at greater than about 80% of said targeted dose.

For the purposes of convenience, methods according to this paragraph are referred to as Pharmaceutical Delivery Method 6A1.

The present invention includes methods for delivering a dose of beclomethasone comprising:
a. providing in a pMDI comprising:
   i. a canister;
   ii. a formulation comprising ipratropium bromide in solution contained in the canister, said formulation comprising: (1) from about 85% by weight to about 95% by weight of HFO-1234ze(E); (2) from about 5% by weight to about 15% by weight of ethanol; and (3) beclomethasone; and
   iii. a metering valve releasably closing the canister and fluidly connected to an actuator having an orifice diameter of about 0.5 mm or less; and
b. actuating said pMDI to produce an aerosol spray comprising said beclomethasone, wherein the amount of said aerosol spray introduced into the mouth of the user is at greater than about 80% of said targeted dose and wherein the fine particle fraction of said aerosol spray introduced into the mouth of the user is greater than about 30%.

For the purposes of convenience, methods according to this paragraph are referred to as Pharmaceutical Delivery Method 6A2.

The present invention includes methods for delivering a dose of beclomethasone comprising:
a. providing in a pMDI comprising:
   i. a canister;
   ii. a formulation comprising ipratropium bromide in solution contained in the canister, said formulation comprising: (1) from about 85% by weight to about 95% by weight of HFO-1234ze(E); (2) from about 5% by weight to about 15% by weight of ethanol; and (3) beclomethasone; and
   iii. a metering valve releasably closing the canister and fluidly connected to an actuator having an orifice diameter of about 0.5 mm or less; and
b. actuating said pMDI to produce an aerosol spray comprising said beclomethasone, wherein the amount of said aerosol spray introduced into the mouth of the user is at greater than about 80% of said targeted dose and wherein the fine particle fraction of said aerosol spray introduced into the mouth of the user is greater than about 35%.

For the purposes of convenience, methods according to this paragraph are referred to as Pharmaceutical Delivery Method 6A3.

The present invention includes methods for delivering a dose of beclomethasone comprising:
a. providing in a pMDI comprising:
   i. a canister;
   ii. a formulation comprising ipratropium bromide in solution contained in the canister, said formulation comprising: (1) from about 85% by weight to about 95% by weight of HFO-1234ze(E); (2) from about 5% by weight to about 15% by weight of ethanol; and (3) beclomethasone; and
   iii. a metering valve releasably closing the canister and fluidly connected to an actuator having an orifice diameter of about 0.5 mm or less; and
b. actuating said pMDI to produce an aerosol spray comprising said beclomethasone, wherein the amount of said aerosol spray introduced into the mouth of the user is at greater than about 80% of said targeted dose and wherein the fine particle fraction of said aerosol spray introduced into the mouth of the user is greater than about 60%.

For the purposes of convenience, methods according to this paragraph are referred to as Pharmaceutical Delivery Method 6A4.

The present invention includes methods for delivering a dose of beclomethasone comprising:
a. providing in a pMDI comprising:
   i. a canister;
   ii. a formulation comprising ipratropium bromide in solution contained in the canister, said formulation comprising: (1) from about 85% by weight to about 95% by weight of HFO-1234ze(E); (2) from about 5% by weight to about 15% by weight of ethanol; and (3) beclomethasone; and
   iii. a metering valve releasably closing the canister and fluidly connected to an actuator having an orifice diameter of about 0.5 mm or less; and
b. actuating said pMDI to produce an aerosol spray comprising said beclomethasone, wherein the amount of said aerosol spray introduced into the mouth of the user is at greater than about 80% of said targeted dose and wherein the fine particle fraction of said aerosol spray introduced into the mouth of the user is greater than about 70%.

For the purposes of convenience, methods according to this paragraph are referred to as Pharmaceutical Delivery Method 6A4.

The present invention includes methods for delivering a dose of beclomethasone comprising:
a. providing in a pMDI comprising:
   i. a canister;
   ii. a formulation comprising ipratropium bromide in solution contained in the canister, said formulation comprising: (1) from about 85% by weight to about 95% by weight of HFO-1234ze(E); (2) from about 5% by weight to about 15% by weight of ethanol; and (3) beclomethasone; and
   iii. a metering valve releasably closing the canister and fluidly connected to an actuator having an orifice diameter of about 0.5 mm or less; and
b. actuating said pMDI to produce an aerosol spray comprising said beclomethasone, wherein the amount of said aerosol spray introduced into the mouth of the user is at greater than about 80% of said targeted dose and wherein the fine particle fraction of said aerosol spray introduced into the mouth of the user is greater than about 70%.

For the purposes of convenience, methods according to this paragraph are referred to as Pharmaceutical Delivery Method 6A4.

The present invention includes methods for delivering a dose of beclomethasone comprising:
a. providing in a pMDI comprising:
   i. a canister;
   ii. a formulation comprising beclomethasone in solution contained in the canister, said formulation comprising: (1) from about 85% by weight to about 95% by weight of HFO-1234ze(E); (2) from about 5% by weight to about 15% by weight of ethanol; and (3) beclomethasone; and
   iii. a metering valve releasably closing the canister and fluidly connected to an actuator having an orifice diameter of about 0.5 mm or less; and
b. actuating said pMDI to produce an aerosol spray comprising said beclomethasone, wherein the amount of said aerosol spray introduced into the mouth of the user is at least about 85% of said targeted dose.

For the purposes of convenience, methods according to this paragraph are referred to as Pharmaceutical Delivery Method 6B.

The present invention includes methods for delivering a dose of beclomethasone comprising:
a. providing in a pMDI comprising:
   i. a canister;
   ii. a formulation comprising beclomethasone in solution contained in the canister, said formulation comprising: (1) from about 85% by weight to about 95% by weight of HFO-1234ze(E); (2) from about 5% by weight to about 15% by weight of ethanol; and (3) beclomethasone; and
   iii. a metering valve releasably closing the canister and fluidly connected to an actuator having an orifice diameter of about 0.5 mm or less; and
b. actuating said pMDI to produce an aerosol spray comprising said beclomethasone, wherein the amount of said aerosol spray introduced into the mouth of the user is at least about 90% of said targeted dose.

For the purposes of convenience, methods according to this paragraph are referred to as Pharmaceutical Delivery Method 6C.

The present invention includes methods for delivering a dose of beclomethasone comprising:
a. providing in a pMDI comprising:
   i. a canister;
   ii. a formulation comprising beclomethasone in solution contained in the canister, said formulation comprising: (1) from about 85% by weight to about 95% by weight of HFO-1234ze(E); (2) from about 5% by weight to about 15% by weight of ethanol; and (3) glycopyrrolate; and
   iii. a metering valve releasably closing the canister and fluidly connected to an actuator having an orifice diameter of about 0.5 mm or less; and
b. actuating said pMDI to produce an aerosol spray comprising said beclomethasone, wherein the amount of said aerosol spray introduced into the mouth of the user is at least about 95% of said targeted dose.

For the purposes of convenience, methods according to this paragraph are referred to as Pharmaceutical Delivery Method 6D.

The present invention also includes a pressurized metered dose inhaler (pMDI) for producing a targeted dose of a pharmaceutical composition, said pMDI comprising:
a. a container;
b. a pharmaceutical formulation under pressure in the container, said pharmaceutical formulation comprising: (i) about from about 75% by weight to about 95% by weight of HFO-1234ze(E); (ii) from about 5% by weight to about 25% by weight of ethanol; and (iii) at least one API in solution in said HFO-1234ze and/or said ethanol;
c. a normally closed valve on the container;
d. an actuator fluidly connected to said normally closed valve and having an actuator orifice with a diameter of not greater than 0.5 mm, said normally closed valve being actuatable by said actuator to an open position to release an aerosol spray containing said API in solution from said container, wherein upon actuating said pMDI to produce said aerosol spray the amount of said aerosol spray leaving the actuator is greater than 80% of the amount of said targeted dose.

For the purposes of convenience, pMDIs according to this paragraph are referred to as MDI1A.

The present invention also includes a pressurized metered dose inhaler (pMDI) for producing a targeted dose of a pharmaceutical composition, said pMDI comprising:
a. a container;
b. a pharmaceutical formulation under pressure in the container, said pharmaceutical formulation comprising: (i) about from about 75% by weight to about 95% by weight of HFO-1234ze(E); (ii) from about 5% by weight to about 25% by weight of ethanol; and (iii) at least one API in solution in said HFO-1234ze and/or said ethanol;
c. a normally closed valve on the container;
d. an actuator fluidly connected to said normally closed valve and having an actuator orifice with a diameter of not greater than 0.5 mm, said normally closed valve being actuatable by said actuator to an open position to release an aerosol spray containing said API in solution from said container, wherein upon actuating said pMDI to produce said aerosol spray the amount of said aerosol spray leaving the actuator is about 85% or greater than the amount of said targeted dose.

For the purposes of convenience, pMDIs according to this paragraph are referred to as MDI1B.

The present invention also includes a pressurized metered dose inhaler (pMDI) for producing a targeted dose of a pharmaceutical composition, said pMDI comprising:
a. a container;
b. a pharmaceutical formulation under pressure in the container, said pharmaceutical formulation comprising: (i) about from about 75% by weight to about 95% by weight of HFO-1234ze(E); (ii) from about 5% by weight to about 25% by weight of ethanol; and (iii) at least one API in solution in said HFO-1234ze and/or said ethanol;
c. a normally closed valve on the container;
d. an actuator fluidly connected to said normally closed valve and having an actuator orifice with a diameter of not greater than 0.5 mm, said normally closed valve being actuatable by said actuator to an open position to release an aerosol spray containing said API in solution from said container, wherein upon actuating said pMDI to produce said aerosol spray the amount of said aerosol spray leaving the actuator is about 90% or greater than the amount of said targeted dose.

For the purposes of convenience, pMDIs according to this paragraph are referred to as MDI1C.

The present invention also includes a pressurized metered dose inhaler (pMDI) for producing a targeted dose of a pharmaceutical composition, said pMDI comprising:
a. a container;
b. a pharmaceutical formulation under pressure in the container, said pharmaceutical formulation comprising: (i) about from about 75% by weight to about 95% by weight of HFO-1234ze(E); (ii) from about 5% by weight to about 25% by weight of ethanol; and (iii) at least one API in solution in said HFO-1234ze and/or said ethanol;
c. a normally closed valve on the container;
d. an actuator fluidly connected to said normally closed valve and having an actuator orifice with a diameter of not greater than 0.5 mm, said normally closed valve being actuatable by said actuator to an open position to release an aerosol spray containing said API in solution from said container, wherein upon actuating said pMDI to produce said aerosol spray the amount of said aerosol spray leaving the actuator is about 95% or greater than the amount of said targeted dose.

For the purposes of convenience, pMDIs according to this paragraph are referred to as MDI1D.

The present invention also includes a pressurized metered dose inhaler (pMDI) for producing a targeted dose of a pharmaceutical composition, said pMDI comprising:
a. a container;
b. a pharmaceutical formulation under pressure in the container, said pharmaceutical formulation comprising: (i) about from about 75% by weight to about 95% by weight of HFO-1234ze(E); (ii) from about 5% by weight to about 25% by weight of ethanol; and (iii) at least one API in solution in said HFO-1234ze and/or said ethanol, said API selected from the group consisting of ipratropium, beclomethasone, formoterol, glycopyrrolate and combinations of two or more of these;
c. a normally closed valve on the container;
d. an actuator fluidly connected to said normally closed valve and having an actuator orifice with a diameter of not greater than 0.5 mm, said normally closed valve being actuatable by said actuator to an open position to release an aerosol spray containing said API in solution from said container, wherein upon actuating said pMDI to produce said aerosol spray the amount of said aerosol spray leaving the actuator is greater than 80% of the amount of said targeted dose.

For the purposes of convenience, pMDIs according to this paragraph are referred to as MDI2A.

The present invention also includes a pressurized metered dose inhaler (pMDI) for producing a targeted dose of a pharmaceutical composition, said pMDI comprising:
a. a container;
b. a pharmaceutical formulation under pressure in the container, said pharmaceutical formulation comprising: (i) about from about 75% by weight to about 95% by weight of HFO-1234ze(E); (ii) from about 5% by weight to about 25% by weight of ethanol; and (iii) at least one API in solution in said HFO-1234ze and/or said ethanol said API selected from the group consisting of ipratropium, beclomethasone, formoterol, glycopyrrolate and combinations of two or more of these;
c. a normally closed valve on the container;
d. an actuator fluidly connected to said normally closed valve and having an actuator orifice with a diameter of not greater than 0.5 mm, said normally closed valve being actuatable by said actuator to an open position to release an aerosol spray containing said API in solution from said container, wherein upon actuating said pMDI to produce said aerosol spray the amount of said aerosol spray leaving the actuator is about 85% or greater than the amount of said targeted dose.

For the purposes of convenience, pMDIs according to this paragraph are referred to as MDI2B.

The present invention also includes a pressurized metered dose inhaler (pMDI) for producing a targeted dose of a pharmaceutical composition, said pMDI comprising:
a. a container;
b. a pharmaceutical formulation under pressure in the container, said pharmaceutical formulation comprising: (i) about from about 75% by weight to about 95% by weight of HFO-1234ze(E); (ii) from about 5% by weight to about 25% by weight of ethanol; and (iii) at least one API in solution in said HFO-1234ze and/or said ethanol, said API selected from the group consisting of ipratropium, beclomethasone, formoterol, glycopyrrolate and combinations of two or more of these;
c. a normally closed valve on the container;
d. an actuator fluidly connected to said normally closed valve and having an actuator orifice with a diameter of not greater than 0.5 mm, said normally closed valve being actuatable by said actuator to an open position to release an aerosol spray containing said API in solution from said container, wherein upon actuating said pMDI to produce said aerosol spray the amount of said aerosol spray leaving the actuator is about 90% or greater than the amount of said targeted dose.

For the purposes of convenience, pMDIs according to this paragraph are referred to as MDI2C.

The present invention also includes a pressurized metered dose inhaler (pMDI) for producing a targeted dose of a pharmaceutical composition, said pMDI comprising:
a. a container;
b. a pharmaceutical formulation under pressure in the container, said pharmaceutical formulation comprising: (i) about from about 75% by weight to about 95% by weight of HFO-1234ze(E); (ii) from about 5% by weight to about 25% by weight of ethanol; and (iii) at least one API in solution in said HFO-1234ze and/or said ethanol, said API selected from the group consisting of ipratropium, beclomethasone, formoterol, glycopyrrolate and combinations of two or more of these;
c. a normally closed valve on the container;
d. an actuator fluidly connected to said normally closed valve and having an actuator orifice with a diameter of not greater than 0.5 mm, said normally closed valve being actuatable by said actuator to an open position to release an aerosol spray containing said API in solution from said container, wherein upon actuating said pMDI to produce said aerosol spray the amount of said aerosol spray leaving the actuator is about 95% or greater than the amount of said targeted dose.

For the purposes of convenience, pMDIs according to this paragraph are referred to as MDI2D.

The present invention includes pharmaceutical compositions in the form of an aerosol spray comprising particles of API selected from the group consisting of ipratropium, beclomethasone, formoterol, glycopyrrolate and combinations of two or more of these and wherein greater than about 40% by weight of said spray of particles are particles having a size of 5 microns or less. For the purposes of convenience, pharmaceutical compositions according to this paragraph are referred to as Pharmaceutical Composition 1.

The present invention includes pharmaceutical compositions in the form of an aerosol spray comprising particles of API selected from the group consisting of ipratropium, beclomethasone, formoterol, glycopyrrolate and combinations of two or more of these and wherein greater than about 45% by weight of said spray of particles are particles having a size of 5 microns or less. For the purposes of convenience, pharmaceutical compositions according to this paragraph are referred to as Pharmaceutical Composition 2.

The present invention includes pharmaceutical compositions in the form of an aerosol spray comprising particles of API selected from the group consisting of ipratropium, beclomethasone, formoterol, glycopyrrolate and combinations of two or more of these and wherein greater than about 50% by weight of said spray of particles are particles having a size of 5 microns or less. For the purposes of convenience, pharmaceutical compositions according to this paragraph are referred to as Pharmaceutical Composition 3.

The present invention includes pharmaceutical compositions in the form of an aerosol spray comprising particles of API selected from the group consisting of ipratropium, beclomethasone, formoterol, glycopyrrolate and combinations of two or more of these and wherein at least about 60% by weight of said spray of particles are particles having a size of 5 microns or less. For the purposes of convenience, pharmaceutical compositions according to this paragraph are referred to as Pharmaceutical Composition 4.

The present invention includes pharmaceutical compositions in the form of an aerosol spray comprising particles of API selected from the group consisting of ipratropium, beclomethasone, formoterol, glycopyrrolate and combinations of two or more of these and wherein at least about 75% by weight of said spray of particles are particles having a size of 5 microns or less. For the purposes of convenience, pharmaceutical compositions according to this paragraph are referred to as Pharmaceutical Composition 5.

### BRIEF DESCRIPTION OF THE DRAWINGS

The invention will now be described with reference to the accompanying drawings in which:

FIG. 1 is a cross-sectional side view of an inhaler including a canister containing a valve and an actuator according to the present disclosure.

FIG. 2 is a detailed cross-sectional side view of the inhaler of FIG. 1.

FIG. 3 is a cross-sectional side view of a metering valve for an inhaler.

FIG. 4 is a graphical representation of the data from Example 2A-2C.

FIG. 5 is a graphical representation of the data from Example 5A.

FIG. 6 is a graphical representation of the data from Example 5B.

FIG. 7 is a graphical representation of the data from Example 5C.

### DETAILED DESCRIPTION OF PREFERRED EMBODIMENTS

### I. Definitions

For the purposes of this invention, the term “about” in relation to the amounts expressed in weight percent means that the amount of the component can vary by an amount of +/−10% on a relative basis by weight. Thus, for example, an amount specified as “about 10%” means the amount can vary from 9% to 11%, and amount specified as about 5% can vary from 4.5% to 5.5%.

For the purposes of this invention, the terms “composition” and “formulation” are used in the broad sense to include both single phase compositions and compositions that comprise two or more phases, such as for example may exist inside a pMDI canister or in an aerosol spray.

The terms “pharmaceutical composition” and “pharmaceutical formulation” are used herein to include any composition or formulation which comprises at least one agent, ingredient, drug, compound, composition, or other substance that may be used on, or administered to, a human or animal for a purpose that includes one or more of therapeutic, pharmaceutical, pharmacological, diagnostic, and prophylactic and immunomodulation.

The term “targeted dose” and its abbreviation “TD” refers to the amount of an API contained in the volume of composition that exits the valve on the canister of a pMDI.

The term “delivered dose” and its abbreviation “DD” refers to the amount of an API contained in the volume of composition that exits the actuator nozzle of a pMDI.

The term or designation DD % means the delivered dose as a percentage of the targeted dose.

The term “fine particle dose” and its abbreviation “FPD” refers to the dose contained in the volume of composition that exits as a single spray from the actuator nozzle of an MDI, in total mass, that is within a respirable range. The dose that is within the respirable range is measured in vitro to be the sum of the dose delivered at stages 2-7 of an Andersen Cascade Impactor operated at a flow rate of 28.3 L/min.

In the context of a composition containing or providing respirable aggregates, particles, drops, etc., such as compositions described herein, the term “fine particle fraction” and its abbreviation “FPF” refers to the proportion, reported as a percentage, of the delivered material relative to the delivered dose (i.e., the amount that exits the actuator of a delivery device, such as an MDI) that is within a respirable range. The amount of delivered material within the respirable range is measured in vitro as the sum of the material delivered at stages 2-7 of an Andersen Cascade Impactor operated at a flow rate of 28.3 L/min. This term as used herein refers to a formulation that is delivered as formulated, that is, not subjected to accelerated aging conditions.

“Mass median aerodynamic diameter” or “MMAD” as used herein refers to the aerodynamic diameter of an aerosol below which 50% of the mass of the aerosol consists of particles with an aerodynamic diameter smaller than the MMAD, with the MMAD being calculated according to monograph 601 of the United States Pharmacopeia (“USP).

As used herein, the term “carrier” refers to one or more pharmacologically inert substances which provide a continuous phase in which ipratropium (including ipratropium bromide) is solvated and/or carried and which comprise components that exert a sufficiently high vapor pressure at normal room temperature to propel the particles from the canister of a pMDI to a patient upon actuation of the MDI's metering valve. Therefore, the term “carrier” encompasses both a single component and a combination of two or more different components that form the medium in which the ipratropium is solvated or otherwise carried. Thus, the HFO-1234ze(E) component of the carrier of present composition acts at least as a propellent.

The term “respirable” generally refers to particles, aggregates, drops, etc. sized such that they can be inhaled and reach the airways of the lung, and is generally understood to refer to particles having a size of about 5 microns or less.

The terms “HFC-134a” and “134a” each mean 1,1,1,2-tetrafluoroethane.

The terms “HFO-1234ze(E),” and “1234ze(E)” as used herein each mean trans-1,3,3,3-tetrafluoropropene. Unless otherwise stated, “HFO-1234ze” and “1234ze” mean trans-1,3,3,3-tetrafluoropropene.

The term “ipratropium” as used herein encompasses any and all pharmaceutically acceptable versions of ipratropium, including pharmaceutically acceptable salts of ipratropium (such as ipratropium bromide).

The term “beclomethasone” as used herein encompasses any and all pharmaceutically acceptable versions of beclamethasone, including pharmaceutically acceptable salts of beclomethasone (such as beclomethasone dipropionate).

The term “formoterol” as used herein encompasses any and all pharmaceutically acceptable versions of formoterol, including pharmaceutically acceptable salts of formoterol (such as formoterol fumarate).

The term “glycopyrrolate” as used herein encompasses any and all pharmaceutically acceptable versions of glycopyrrolate (such as glycopyrrolate bromide). For clarity, the terms “glycopyrrolate” and “glycopyrronium” are considered synonymous.

Reference herein to a group of defined items includes all such defined items, including all such items with suffix designations. Thus, for example, a reference herein to “MDI1” is a specific reference to each of MDI1A, MDI1B, MDI1C, MDI1D and MDI1E.

### II. The Compositions

The preferred pharmaceutical compositions of the present invention, including each of Pharmaceutical Compositions 1-5, preferably comprise solutions containing one or more APIs in solution in one or more components of the carrier, including particularly one or both of HFO-1234ze(E) and ethanol.

The preferred pharmaceutical compositions of the present invention, including each of Pharmaceutical Compositions 1-5, are physically stable.

The preferred pharmaceutical compositions of the present invention, including each of Pharmaceutical Compositions 1-5, are chemically stable.

The preferred pharmaceutical compositions of the present invention, including each of Pharmaceutical Compositions 1-5, are physically stable and chemically stable.

The concentration of the components in the present compositions can generally vary widely within the broad scope of the present invention.

For all compositions of the present invention, other than those defined as “consisting of” the designated components, including each of Pharmaceutical Compositions 1-5, additional components or excipients may be present. These components may have various uses and functions, including, but not limited to, facilitating formation of the solution, stabilizing the solution, and/or aiding in chemical stabilization of ipratropium or other components.

Preferred excipients include are suitable for inhaled delivery and do not substantially degrade the solution.

For all compositions of the present invention, including each of Pharmaceutical Compositions 1-5, the composition comprises a solution of the indicated API in the HFO-1234ze(E) and/or ethanol. As used herein, the term solution means that essentially all of the indicted API is in solution.

In certain preferred forms, the compositions of the present invention, including each of Pharmaceutical Compositions 1-5, have a Global Warming Potential (GWP) of not greater than about 300, more preferably not greater than about 150, not greater than 75, and most preferably not greater than about 10. As used herein, “GWP” is measured relative to that of carbon dioxide and over a 100-year time horizon, as defined in “The Scientific Assessment of Ozone Depletion, 2002, a report of the World Meteorological Association's Global Ozone Research and Monitoring Project,” which is incorporated herein by reference.

In certain preferred forms, the present compositions also preferably have an Ozone Depletion Potential (ODP) of not greater than 0.05, more preferably not greater than 0.02 and even more preferably about zero. As used herein, “ODP” is as defined in “The Scientific Assessment of Ozone Depletion, 2002, A report of the World Meteorological Association's Global Ozone Research and Monitoring Project,” which is incorporated herein by reference.

The preferred composition or formulation of the present of the present invention contained in the canister of the present pMDIs, including each of MDI1 through MDI6, comprises from about 75% to about 95% by weight of HFO-1234ze(E), from about 5% to about 25% of ethanol, and from about 0.01 wt % about 0.06 wt % of API. Particular formulations to be included in the present pMDIs, including each of MDI1 through MDI6, include those identified in the following Table PF for “pharmaceutical formulation,” wherein the first column of the table includes “PF” as an abbreviation for a defined Pharmaceutical Formulation. In Table 1 below the following terms have the following meaning:
“IP-AS” means any pharmaceutically effective salt of ipratropium.
“IB” means ipratropium bromide, whether added to the formulation as anhydrous ipratropium bromide or as ipratropium bromide monohydrate;
“BM-AS” means any pharmaceutically effective salt of beclomethasone.
“BM-DP” means beclomethasone dipropionate.
“F-AS” means any pharmaceutically effective salt of formoterol.
“FF” means formoterol fumarate.
“G-AS” means any pharmaceutically effective salt of glycopyrrolate.
“GB” means glycopyrrolate bromide.
“ANY” in the column with the heading “API—type or specific” means any API that is in solution in the carrier or any one of its components.
“ANY” in the column with the heading “API—wt % of PC” means any concentration of the API in solution in the carrier or any one of its components.
“NR” means that the component or a particular amount is “not required” according to the specified PF definition and as such its presence in any amount or in no amount is permitted.
“Yes” means the component is required but that any type or amount is permitted.
“Comp” means that the specified pharmaceutical composition comprises the items identified in the table.
“CEO” means that the specified composition consists essentially of the items identified in the table.
“CO” means that composition consists of the items identified in the table.

The column heading Excipient indicates the pharmaceutical composition comprises the type or category of excipient as defined herein.

The column heading Excipient Amount indicates the amount of the indicated excipient as a percentage of the pharmaceutical composition.

All amounts are understood to be preceded by the word “about.”

TABLE PC

| | | Carrier | | | | | |
|---|---|---|---|---|---|---|---|
| | | components, wt % | | | | Excipient | |
| | | of PC | | API | | | Amount, |
| PC | | 1234ze | | Type or | wt % | Type or | wt % |
| No. | | (E) | Ethanol | specific | of PC | specific | of PC |
| 1A | Comp | 80-<90 | >5-20 | ANY | ANY | NR | NR |
| 1B | CEO | 80-<89 | >11-20 | ANY | ANY | NR | NR |
| 1C | Comp | 80-88 | 12-20 | ANY | ANY | NR | NR |
| 1D | CEO | 80-87 | 13-20 | ANY | ANY | NR | NR |
| 1E | Comp | 80-86 | 14-20 | ANY | ANY | NR | NR |
| 1F | CEO | 82-87 | 13-18 | ANY | ANY | NR | NR |
| 1G | Comp | 83-86 | 14-17 | ANY | ANY | NR | NR |
| 1H | CEO | 84-86 | 14-16 | ANY | ANY | NR | NR |
| 1I | Comp | 80-<90 | >5-20 | ANY | ANY | NR | NR |
| 1J | CEO | 80-<89 | >11-20 | ANY | ANY | NR | NR |
| 1K | Comp | 80-88 | 12-20 | ANY | ANY | NR | NR |
| 1L | CEO | 80-87 | 13-20 | ANY | ANY | NR | NR |
| 1M | CEO | 80-86 | 14-20 | ANY | ANY | NR | NR |
| 1N | Comp | 82-87 | 13-18 | ANY | ANY | NR | NR |
| 1O | CEO | 83-86 | 14-17 | ANY | ANY | NR | NR |
| 1P | Comp | 84-86 | 14-16 | ANY | ANY | NR | NR |
| 1Q | CEO | 82-85 | 15-18 | ANY | ANY | NR | NR |
| 1R | Comp | 83-85 | 15-17 | ANY | ANY | NR | NR |
| 1S | CEO | 84 | 16 | ANY | ANY | NR | NR |
| 1T | CEO | 85 | 15 | ANY | ANY | NR | NR |
| 1U | CEO | 86 | 14 | ANY | ANY | NR | NR |
| 2A | Comp | 80-<90 | >5-20 | ANY | 0.01-0.06 | NR | NR |
| 2B | CEO | 80-<89 | >11-20 | ANY | 0.01-0.06 | NR | NR |
| 2C | Comp | 80-88 | 12-20 | ANY | 0.01-0.06 | NR | NR |
| 2D | CEO | 80-87 | 13-20 | ANY | 0.01-0.06 | NR | NR |
| 2E | Comp | 80-86 | 14-20 | ANY | 0.01-0.06 | NR | NR |
| 2F | CEO | 82-87 | 13-18 | ANY | 0.01-0.06 | NR | NR |
| 2G | Comp | 83-86 | 14-17 | ANY | 0.01-0.06 | NR | NR |
| 2H | CEO | 84-86 | 14-16 | ANY | 0.01-0.06 | NR | NR |
| 2I | Comp | 80-<90 | >5-20 | ANY | 0.01-0.06 | NR | NR |
| 2J | CEO | 80-<89 | >11-20 | ANY | 0.01-0.06 | NR | NR |
| 2K | Comp | 80-88 | 12-20 | ANY | 0.01-0.06 | NR | NR |
| 2L | CEO | 80-87 | 13-20 | ANY | 0.01-0.06 | NR | NR |
| 2M | CEO | 80-86 | 14-20 | ANY | 0.01-0.06 | NR | NR |
| 2N | Comp | 82-87 | 13-18 | ANY | 0.01-0.06 | NR | NR |
| 2O | CEO | 83-86 | 14-17 | ANY | 0.01-0.06 | NR | NR |
| 2P | Comp | 84-86 | 14-16 | ANY | 0.01-0.06 | NR | NR |
| 2Q | CEO | 82-85 | 15-18 | ANY | 0.01-0.06 | NR | NR |
| 2R | Comp | 83-85 | 15-17 | ANY | 0.01-0.06 | NR | NR |
| 2S | CEO | 84 | 16 | ANY | 0.01-0.06 | NR | NR |
| 2T | CEO | 85 | 15 | ANY | 0.01-0.06 | NR | NR |
| 2U | CEO | 86 | 14 | ANY | 0.01-0.06 | NR | NR |
| 3A | Comp | 80-<90 | >5-20 | ANY | 0.03-0.04 | NR | NR |
| 3B | CEO | 80-<89 | >11-20 | ANY | 0.03-0.04 | NR | NR |
| 3C | Comp | 80-88 | 12-20 | ANY | 0.03-0.04 | NR | NR |
| 3D | CEO | 80-87 | 13-20 | ANY | 0.03-0.04 | NR | NR |
| 3E | Comp | 80-86 | 14-20 | ANY | 0.03-0.04 | NR | NR |
| 3F | CEO | 82-87 | 13-18 | ANY | 0.03-0.04 | NR | NR |
| 3G | Comp | 83-86 | 14-17 | ANY | 0.03-0.04 | NR | NR |
| 3H | CEO | 84-86 | 14-16 | ANY | 0.03-0.04 | NR | NR |
| 3I | Comp | 80-<90 | >5-20 | ANY | 0.03-0.04 | NR | NR |
| 3J | CEO | 80-<89 | >11-20 | ANY | 0.03-0.04 | NR | NR |
| 3K | Comp | 80-88 | 12-20 | ANY | 0.03-0.04 | NR | NR |
| 3L | CEO | 80-87 | 13-20 | ANY | 0.03-0.04 | NR | NR |
| 3M | CEO | 80-86 | 14-20 | ANY | 0.03-0.04 | NR | NR |
| 3N | Comp | 82-87 | 13-18 | ANY | 0.03-0.04 | NR | NR |
| 3O | CEO | 83-86 | 14-17 | ANY | 0.03-0.04 | NR | NR |
| 3P | Comp | 84-86 | 14-16 | ANY | 0.03-0.04 | NR | NR |
| 3Q | CEO | 82-85 | 15-18 | ANY | 0.03-0.04 | NR | NR |
| 3R | Comp | 83-85 | 15-17 | ANY | 0.03-0.04 | NR | NR |
| 3S | CEO | 84 | 16 | ANY | 0.03-0.04 | NR | NR |
| 3T | CEO | 85 | 15 | ANY | 0.03-0.04 | NR | NR |
| 3U | CEO | 86 | 14 | ANY | 0.03-0.04 | NR | NR |
| 4A | Comp | 80-<90 | >5-20 | IP-AS | 0.01-0.06 | NR | NR |
| 4B | CEO | 80-<89 | >11-20 | IP-AS | 0.01-0.06 | NR | NR |
| 4C | Comp | 80-88 | 12-20 | IP-AS | 0.01-0.06 | NR | NR |
| 4D | CEO | 80-87 | 13-20 | IP-AS | 0.01-0.06 | NR | NR |
| 4E | Comp | 80-86 | 14-20 | IP-AS | 0.01-0.06 | NR | NR |
| 4F | CEO | 82-87 | 13-18 | IP-AS | 0.01-0.06 | NR | NR |
| 4G | Comp | 83-86 | 14-17 | IP-AS | 0.01-0.06 | NR | NR |
| 4H | CEO | 84-86 | 14-16 | IP-AS | 0.01-0.06 | NR | NR |
| 4I | Comp | 80-<90 | >5-20 | IP-AS | 0.01-0.06 | NR | NR |
| 4J | CEO | 80-<89 | >11-20 | IP-AS | 0.01-0.06 | NR | NR |
| 4K | Comp | 80-88 | 12-20 | IP-AS | 0.01-0.06 | NR | NR |
| 4L | CEO | 80-87 | 13-20 | IP-AS | 0.01-0.06 | NR | NR |
| 4M | CEO | 80-86 | 14-20 | IP-AS | 0.01-0.06 | NR | NR |
| 4N | Comp | 82-87 | 13-18 | IP-AS | 0.01-0.06 | NR | NR |
| 4O | CEO | 83-86 | 14-17 | IP-AS | 0.01-0.06 | NR | NR |
| 4P | Comp | 84-86 | 14-16 | IP-AS | 0.01-0.06 | NR | NR |
| 4Q | CEO | 82-85 | 15-18 | IP-AS | 0.01-0.06 | NR | NR |
| 4R | Comp | 83-85 | 15-17 | IP-AS | 0.01-0.06 | NR | NR |
| 4S | CEO | 84 | 16 | IP-AS | 0.01-0.06 | NR | NR |
| 4T | CEO | 85 | 15 | IP-AS | 0.01-0.06 | NR | NR |
| 4U | CEO | 86 | 14 | IP-AS | 0.01-0.06 | NR | NR |
| 5A | Comp | 80-<90 | >5-20 | IP-AS | 0.03-0.04 | NR | NR |
| 5B | CEO | 80-<89 | >11-20 | IP-AS | 0.03-0.04 | NR | NR |
| 5C | Comp | 80-88 | 12-20 | IP-AS | 0.03-0.04 | NR | NR |
| 5D | CEO | 80-87 | 13-20 | IP-AS | 0.03-0.04 | NR | NR |
| 5E | Comp | 80-86 | 14-20 | IP-AS | 0.03-0.04 | NR | NR |
| 5F | CEO | 82-87 | 13-18 | IP-AS | 0.03-0.04 | NR | NR |
| 5G | Comp | 83-86 | 14-17 | IP-AS | 0.03-0.04 | NR | NR |
| 5H | CEO | 84-86 | 14-16 | IP-AS | 0.03-0.04 | NR | NR |
| 5I | Comp | 80-<90 | >5-20 | IP-AS | 0.03-0.04 | NR | NR |
| 5J | CEO | 80-<89 | >11-20 | IP-AS | 0.03-0.04 | NR | NR |
| 5K | Comp | 80-88 | 12-20 | IP-AS | 0.03-0.04 | NR | NR |
| 5L | CEO | 80-87 | 13-20 | IP-AS | 0.03-0.04 | NR | NR |
| 5M | CEO | 80-86 | 14-20 | IP-AS | 0.03-0.04 | NR | NR |
| 5N | Comp | 82-87 | 13-18 | IP-AS | 0.03-0.04 | NR | NR |
| 5O | CEO | 83-86 | 14-17 | IP-AS | 0.03-0.04 | NR | NR |
| 5P | Comp | 84-86 | 14-16 | IP-AS | 0.03-0.04 | NR | NR |
| 5Q | CEO | 82-85 | 15-18 | IP-AS | 0.03-0.04 | NR | NR |
| 5R | Comp | 83-85 | 15-17 | IP-AS | 0.03-0.04 | NR | NR |
| 5S | CEO | 84 | 16 | IP-AS | 0.03-0.04 | NR | NR |
| 5T | CEO | 85 | 15 | IP-AS | 0.03-0.04 | NR | NR |
| 5U | CEO | 86 | 14 | IP-AS | 0.03-0.04 | NR | NR |
| 6A | Comp | 80-<90 | >5-20 | IB | 0.03-0.04 | Citric acid | NR |
| 6B | CEO | 80-<89 | >11-20 | IB | 0.03-0.04 | Citric acid | NR |
| 6C | Comp | 80-88 | 12-20 | IB | 0.03-0.04 | Citric acid | NR |
| 6D | CEO | 80-87 | 13-20 | IB | 0.03-0.04 | Citric acid | NR |
| 6E | Comp | 80-86 | 14-20 | IB | 0.03-0.04 | Citric acid | NR |
| 6F | CEO | 82-87 | 13-18 | IB | 0.03-0.04 | Citric acid | NR |
| 6G | Comp | 83-86 | 14-17 | IB | 0.03-0.04 | Citric acid | NR |
| 6H | CEO | 84-86 | 14-16 | IB | 0.03-0.04 | Citric acid | NR |
| 6I | Comp | 80-<90 | >5-20 | IB | 0.03-0.04 | Citric acid | NR |
| 6J | CEO | 80-<89 | >11-20 | IB | 0.03-0.04 | Citric acid | NR |
| 6K | Comp | 80-88 | 12-20 | IB | 0.03-0.04 | Citric acid | NR |
| 6L | CEO | 80-87 | 13-20 | IB | 0.03-0.04 | Citric acid | NR |
| 6M | CEO | 80-86 | 14-20 | IB | 0.03-0.04 | Citric acid | NR |
| 6N | Comp | 82-87 | 13-18 | IB | 0.03-0.04 | Citric acid | NR |
| 6O | CEO | 83-86 | 14-17 | IB | 0.03-0.04 | Citric acid | NR |
| 6P | Comp | 84-86 | 14-16 | IB | 0.03-0.04 | Citric acid | NR |
| 6Q | CEO | 82-85 | 15-18 | IB | 0.03-0.04 | Citric acid | NR |
| 6R | Comp | 83-85 | 15-17 | IB | 0.03-0.04 | Citric acid | NR |
| 6S | CEO | 84 | 16 | IB | 0.03-0.04 | Citric acid | 0.005-0.015 |
| 6T | CEO | 85 | 15 | IB | 0.03-0.04 | Citric acid | 0.005-0.015 |
| 6U | CEO | 86 | 14 | IB | 0.03-0.04 | Citric acid | 0.005-0.015 |
| 7A | Comp | 80-<90 | >5-20 | IB | 0.01-0.06 | NR | NR |
| 7B | CEO | 80-<89 | >11-20 | IB | 0.01-0.06 | NR | NR |
| 7C | Comp | 80-88 | 12-20 | IB | 0.01-0.06 | NR | NR |
| 7D | CEO | 80-87 | 13-20 | IB | 0.01-0.06 | NR | NR |
| 7E | Comp | 80-86 | 14-20 | IB | 0.01-0.06 | NR | NR |
| 7F | CEO | 82-87 | 13-18 | IB | 0.01-0.06 | NR | NR |
| 7G | Comp | 83-86 | 14-17 | IB | 0.01-0.06 | NR | NR |
| 7H | CEO | 84-86 | 14-16 | IB | 0.01-0.06 | NR | NR |
| 7I | Comp | 80-<90 | >5-20 | IB | 0.01-0.06 | NR | NR |
| 7J | CEO | 80-<89 | >11-20 | IB | 0.01-0.06 | NR | NR |
| 7K | Comp | 80-88 | 12-20 | IB | 0.01-0.06 | NR | NR |
| 7L | CEO | 80-87 | 13-20 | IB | 0.01-0.06 | NR | NR |
| 7M | CEO | 80-86 | 14-20 | IB | 0.01-0.06 | NR | NR |
| 7N | Comp | 82-87 | 13-18 | IB | 0.01-0.06 | NR | NR |
| 7O | CEO | 83-86 | 14-17 | IB | 0.01-0.06 | NR | NR |
| 7P | Comp | 84-86 | 14-16 | IB | 0.01-0.06 | NR | NR |
| 7Q | CEO | 82-85 | 15-18 | IB | 0.01-0.06 | NR | NR |
| 7R | Comp | 83-85 | 15-17 | IB | 0.01-0.06 | NR | NR |
| 7S | CEO | 84 | 16 | IB | 0.01-0.06 | NR | NR |
| 7T | CEO | 85 | 15 | IB | 0.01-0.06 | NR | NR |
| 7U | CEO | 86 | 14 | IB | 0.01-0.06 | NR | NR |
| 8A | Comp | 80-<90 | >5-20 | IB | 0.03-0.04 | NR | NR |
| 8B | CEO | 80-<89 | >11-20 | IB | 0.03-0.04 | NR | NR |
| 8C | Comp | 80-88 | 12-20 | IB | 0.03-0.04 | NR | NR |
| 8D | CEO | 80-87 | 13-20 | IB | 0.03-0.04 | NR | NR |
| 8E | Comp | 80-86 | 14-20 | IB | 0.03-0.04 | NR | NR |
| 8F | CEO | 82-87 | 13-18 | IB | 0.03-0.04 | NR | NR |
| 8G | Comp | 83-86 | 14-17 | IB | 0.03-0.04 | NR | NR |
| 8H | CEO | 84-86 | 14-16 | IB | 0.03-0.04 | NR | NR |
| 8I | Comp | 80-<90 | >5-20 | IB | 0.03-0.04 | NR | NR |
| 8J | CEO | 80-<89 | >11-20 | IB | 0.03-0.04 | NR | NR |
| 8K | Comp | 80-88 | 12-20 | IB | 0.03-0.04 | NR | NR |
| 8L | CEO | 80-87 | 13-20 | IB | 0.03-0.04 | NR | NR |
| 8M | CEO | 80-86 | 14-20 | IB | 0.03-0.04 | NR | NR |
| 8N | Comp | 82-87 | 13-18 | IB | 0.03-0.04 | NR | NR |
| 8O | CEO | 83-86 | 14-17 | IB | 0.03-0.04 | NR | NR |
| 8P | Comp | 84-86 | 14-16 | IB | 0.03-0.04 | NR | NR |
| 8Q | CEO | 82-85 | 15-18 | IB | 0.03-0.04 | NR | NR |
| 8R | Comp | 83-85 | 15-17 | IB | 0.03-0.04 | NR | NR |
| 8S | CEO | 84 | 16 | IB | 0.03-0.04 | NR | NR |
| 8T | CEO | 85 | 15 | IB | 0.03-0.04 | NR | NR |
| 8U | CEO | 86 | 14 | IB | 0.03-0.04 | NR | NR |
| 9A | Comp | 80-<90 | >5-20 | BM-AS | 0.01-0.06 | NR | NR |
| 9B | CEO | 80-<89 | >11-20 | BM-AS | 0.01-0.06 | NR | NR |
| 9C | Comp | 80-88 | 12-20 | BM-AS | 0.01-0.06 | NR | NR |
| 9D | CEO | 80-87 | 13-20 | BM-AS | 0.01-0.06 | NR | NR |
| 9E | Comp | 80-86 | 14-20 | BM-AS | 0.01-0.06 | NR | NR |
| 9F | CEO | 82-87 | 13-18 | BM-AS | 0.01-0.06 | NR | NR |
| 9G | Comp | 83-86 | 14-17 | BM-AS | 0.01-0.06 | NR | NR |
| 9H | CEO | 84-86 | 14-16 | BM-AS | 0.01-0.06 | NR | NR |
| 9I | Comp | 80-<90 | >5-20 | BM-AS | 0.01-0.06 | NR | NR |
| 9J | CEO | 80-<89 | >11-20 | BM-AS | 0.01-0.06 | NR | NR |
| 9K | Comp | 80-88 | 12-20 | BM-AS | 0.01-0.06 | NR | NR |
| 9L | CEO | 80-87 | 13-20 | BM-AS | 0.01-0.06 | NR | NR |
| 9M | CEO | 80-86 | 14-20 | BM-AS | 0.01-0.06 | NR | NR |
| 9N | Comp | 82-87 | 13-18 | BM-AS | 0.01-0.06 | NR | NR |
| 9O | CEO | 83-86 | 14-17 | BM-AS | 0.01-0.06 | NR | NR |
| 9P | Comp | 84-86 | 14-16 | BM-AS | 0.01-0.06 | NR | NR |
| 9Q | CEO | 82-85 | 15-18 | BM-AS | 0.01-0.06 | NR | NR |
| 9R | Comp | 83-85 | 15-17 | BM-AS | 0.01-0.06 | NR | NR |
| 9S | CEO | 84 | 16 | BM-AS | 0.01-0.06 | NR | NR |
| 9T | CEO | 85 | 15 | BM-AS | 0.01-0.06 | NR | NR |
| 9U | CEO | 86 | 14 | BM-AS | 0.01-0.06 | NR | NR |
| 10A | Comp | 80-<90 | >5-20 | BM-AS | 0.03-0.04 | NR | NR |
| 10B | CEO | 80-<89 | >11-20 | BM-AS | 0.03-0.04 | NR | NR |
| 10C | Comp | 80-88 | 12-20 | BM-AS | 0.03-0.04 | NR | NR |
| 10D | CEO | 80-87 | 13-20 | BM-AS | 0.03-0.04 | NR | NR |
| 10E | Comp | 80-86 | 14-20 | BM-AS | 0.03-0.04 | NR | NR |
| 10F | CEO | 82-87 | 13-18 | BM-AS | 0.03-0.04 | NR | NR |
| 10G | Comp | 83-86 | 14-17 | BM-AS | 0.03-0.04 | NR | NR |
| 10H | CEO | 84-86 | 14-16 | BM-AS | 0.03-0.04 | NR | NR |
| 10I | Comp | 80-<90 | >5-20 | BM-AS | 0.03-0.04 | NR | NR |
| 10J | CEO | 80-<89 | >11-20 | BM-AS | 0.03-0.04 | NR | NR |
| 10K | Comp | 80-88 | 12-20 | BM-AS | 0.03-0.04 | NR | NR |
| 10L | CEO | 80-87 | 13-20 | BM-AS | 0.03-0.04 | NR | NR |
| 10M | CEO | 80-86 | 14-20 | BM-AS | 0.03-0.04 | NR | NR |
| 10N | Comp | 82-87 | 13-18 | BM-AS | 0.03-0.04 | NR | NR |
| 10O | CEO | 83-86 | 14-17 | BM-AS | 0.03-0.04 | NR | NR |
| 10P | Comp | 84-86 | 14-16 | BM-AS | 0.03-0.04 | NR | NR |
| 10Q | CEO | 82-85 | 15-18 | BM-AS | 0.03-0.04 | NR | NR |
| 10R | Comp | 83-85 | 15-17 | BM-AS | 0.03-0.04 | NR | NR |
| 10S | CEO | 84 | 16 | BM-AS | 0.03-0.04 | NR | NR |
| 10T | CEO | 85 | 15 | BM-AS | 0.03-0.04 | NR | NR |
| 10U | CEO | 86 | 14 | BM-AS | 0.03-0.04 | NR | NR |
| 11A | Comp | 80-<90 | >5-20 | BM-DP | 0.03-0.04 | Oleic Acid | NR |
| 11B | CEO | 80-<89 | >11-20 | BM-DP | 0.03-0.04 | Oleic Acid | NR |
| 11C | Comp | 80-88 | 12-20 | BM-DP | 0.03-0.04 | Oleic Acid | NR |
| 11D | CEO | 80-87 | 13-20 | BM-DP | 0.03-0.04 | Oleic Acid | NR |
| 11E | Comp | 80-86 | 14-20 | BM-DP | 0.03-0.04 | Oleic Acid | NR |
| 11F | CEO | 82-87 | 13-18 | BM-DP | 0.03-0.04 | Oleic Acid | NR |
| 11G | Comp | 83-86 | 14-17 | BM-DP | 0.03-0.04 | Oleic Acid | NR |
| 11H | CEO | 84-86 | 14-16 | BM-DP | 0.03-0.04 | Oleic Acid | NR |
| 11I | Comp | 80-<90 | >5-20 | BM-DP | 0.03-0.04 | Oleic Acid | NR |
| 11J | CEO | 80-<89 | >11-20 | BM-DP | 0.03-0.04 | Oleic Acid | NR |
| 11K | Comp | 80-88 | 12-20 | BM-DP | 0.03-0.04 | Oleic Acid | NR |
| 11L | CEO | 80-87 | 13-20 | BM-DP | 0.03-0.04 | Oleic Acid | NR |
| 11M | CEO | 80-86 | 14-20 | BM-DP | 0.03-0.04 | Oleic Acid | NR |
| 11N | Comp | 82-87 | 13-18 | BM-DP | 0.03-0.04 | Oleic Acid | NR |
| 11O | CEO | 83-86 | 14-17 | BM-DP | 0.03-0.04 | Oleic Acid | NR |
| 11P | Comp | 84-86 | 14-16 | BM-DP | 0.03-0.04 | Oleic Acid | NR |
| 11Q | CEO | 82-85 | 15-18 | BM-DP | 0.03-0.04 | Oleic Acid | NR |
| 11R | Comp | 83-85 | 15-17 | BM-DP | 0.03-0.04 | Oleic Acid | NR |
| 11S | CEO | 84 | 16 | BM-DP | 0.03-0.04 | Oleic Acid | 0.005-0.015 |
| 11T | CEO | 85 | 15 | BM-DP | 0.03-0.04 | Oleic Acid | 0.005-0.015 |
| 11U | CEO | 86 | 14 | BM-DP | 0.03-0.04 | Oleic Acid | 0.005-0.015 |
| 12A | Comp | 80-<90 | >5-20 | BM-DP | 0.01-0.06 | NR | NR |
| 12B | CEO | 80-<89 | >11-20 | BM-DP | 0.01-0.06 | NR | NR |
| 12C | Comp | 80-88 | 12-20 | BM-DP | 0.01-0.06 | NR | NR |
| 12D | CEO | 80-87 | 13-20 | BM-DP | 0.01-0.06 | NR | NR |
| 12E | Comp | 80-86 | 14-20 | BM-DP | 0.01-0.06 | NR | NR |
| 12F | CEO | 82-87 | 13-18 | BM-DP | 0.01-0.06 | NR | NR |
| 12G | Comp | 83-86 | 14-17 | BM-DP | 0.01-0.06 | NR | NR |
| 12H | CEO | 84-86 | 14-16 | BM-DP | 0.01-0.06 | NR | NR |
| 12I | Comp | 80-<90 | >5-20 | BM-DP | 0.01-0.06 | NR | NR |
| 12J | CEO | 80-<89 | >11-20 | BM-DP | 0.01-0.06 | NR | NR |
| 12K | Comp | 80-88 | 12-20 | BM-DP | 0.01-0.06 | NR | NR |
| 12L | CEO | 80-87 | 13-20 | BM-DP | 0.01-0.06 | NR | NR |
| 12M | CEO | 80-86 | 14-20 | BM-DP | 0.01-0.06 | NR | NR |
| 12N | Comp | 82-87 | 13-18 | BM-DP | 0.01-0.06 | NR | NR |
| 12O | CEO | 83-86 | 14-17 | BM-DP | 0.01-0.06 | NR | NR |
| 12P | Comp | 84-86 | 14-16 | BM-DP | 0.01-0.06 | NR | NR |
| 12Q | CEO | 82-85 | 15-18 | BM-DP | 0.01-0.06 | NR | NR |
| 12R | Comp | 83-85 | 15-17 | BM-DP | 0.01-0.06 | NR | NR |
| 12S | CEO | 84 | 16 | BM-DP | 0.01-0.06 | NR | NR |
| 12T | CEO | 85 | 15 | BM-DP | 0.01-0.06 | NR | NR |
| 12U | CEO | 86 | 14 | BM-DP | 0.01-0.06 | NR | NR |
| 13A | Comp | 80-<90 | >5-20 | BM-DP | 0.03-0.04 | NR | NR |
| 13B | CEO | 80-<89 | >11-20 | BM-DP | 0.03-0.04 | NR | NR |
| 13C | Comp | 80-88 | 12-20 | BM-DP | 0.03-0.04 | NR | NR |
| 13D | CEO | 80-87 | 13-20 | BM-DP | 0.03-0.04 | NR | NR |
| 13E | Comp | 80-86 | 14-20 | BM-DP | 0.03-0.04 | NR | NR |
| 13F | CEO | 82-87 | 13-18 | BM-DP | 0.03-0.04 | NR | NR |
| 13G | Comp | 83-86 | 14-17 | BM-DP | 0.03-0.04 | NR | NR |
| 13H | CEO | 84-86 | 14-16 | BM-DP | 0.03-0.04 | NR | NR |
| 13I | Comp | 80-<90 | >5-20 | BM-DP | 0.03-0.04 | NR | NR |
| 13J | CEO | 80-<89 | >11-20 | BM-DP | 0.03-0.04 | NR | NR |
| 13K | Comp | 80-88 | 12-20 | BM-DP | 0.03-0.04 | NR | NR |
| 13L | CEO | 80-87 | 13-20 | BM-DP | 0.03-0.04 | NR | NR |
| 13M | CEO | 80-86 | 14-20 | BM-DP | 0.03-0.04 | NR | NR |
| 13N | Comp | 82-87 | 13-18 | BM-DP | 0.03-0.04 | NR | NR |
| 13O | CEO | 83-86 | 14-17 | BM-DP | 0.03-0.04 | NR | NR |
| 13P | Comp | 84-86 | 14-16 | BM-DP | 0.03-0.04 | NR | NR |
| 13Q | CEO | 82-85 | 15-18 | BM-DP | 0.03-0.04 | NR | NR |
| 13R | Comp | 83-85 | 15-17 | BM-DP | 0.03-0.04 | NR | NR |
| 13S | CEO | 84 | 16 | BM-DP | 0.03-0.04 | NR | NR |
| 13T | CEO | 85 | 15 | BM-DP | 0.03-0.04 | NR | NR |
| 13U | CEO | 86 | 14 | BM-DP | 0.03-0.04 | NR | NR |
| 14A | Comp | 80-<90 | >5-20 | BM-DP | 0.03-0.04 | Oleic Acid | NR |
| 14B | CEO | 80-<89 | >11-20 | BM-DP | 0.03-0.04 | Oleic Acid | NR |
| 14C | Comp | 80-88 | 12-20 | BM-DP | 0.03-0.04 | Oleic Acid | NR |
| 14D | CEO | 80-87 | 13-20 | BM-DP | 0.03-0.04 | Oleic Acid | NR |
| 14E | Comp | 80-86 | 14-20 | BM-DP | 0.03-0.04 | Oleic Acid | NR |
| 14F | CEO | 82-87 | 13-18 | BM-DP | 0.03-0.04 | Oleic Acid | NR |
| 14G | Comp | 83-86 | 14-17 | BM-DP | 0.03-0.04 | Oleic Acid | NR |
| 14H | CEO | 84-86 | 14-16 | BM-DP | 0.03-0.04 | Oleic Acid | NR |
| 14I | Comp | 80-<90 | >5-20 | BM-DP | 0.03-0.04 | Oleic Acid | NR |
| 14J | CEO | 80-<89 | >11-20 | BM-DP | 0.03-0.04 | Oleic Acid | NR |
| 14K | Comp | 80-88 | 12-20 | BM-DP | 0.03-0.04 | Oleic Acid | NR |
| 14L | CEO | 80-87 | 13-20 | BM-DP | 0.03-0.04 | Oleic Acid | NR |
| 14M | CEO | 80-86 | 14-20 | BM-DP | 0.03-0.04 | Oleic Acid | NR |
| 14N | Comp | 82-87 | 13-18 | BM-DP | 0.03-0.04 | Oleic Acid | NR |
| 14O | CEO | 83-86 | 14-17 | BM-DP | 0.03-0.04 | Oleic Acid | NR |
| 14P | Comp | 84-86 | 14-16 | BM-DP | 0.03-0.04 | Oleic Acid | NR |
| 14Q | CEO | 82-85 | 15-18 | BM-DP | 0.03-0.04 | Oleic Acid | NR |
| 14R | Comp | 83-85 | 15-17 | BM-DP | 0.03-0.04 | Oleic Acid | NR |
| 14S | CEO | 84 | 16 | BM-DP | 0.03-0.04 | Oleic Acid | 0.005-0.015 |
| 14T | CEO | 85 | 15 | BM-DP | 0.03-0.04 | Oleic Acid | 0.005-0.015 |
| 14U | CEO | 86 | 14 | BM-DP | 0.03-0.04 | Oleic Acid | 0.005-0.015 |
| 15A | Comp | 80-<90 | >14-20 | F-AS | 0.01-0.06 | NR | NR |
| 15B | CEO | 80-<89 | >15-20 | F-AS | 0.01-0.06 | NR | NR |
| 15C | Comp | 80-88 | 12-20 | F-AS | 0.01-0.06 | NR | NR |
| 15D | CEO | 80-87 | 13-20 | F-AS | 0.01-0.06 | NR | NR |
| 15E | Comp | 80-86 | 14-20 | F-AS | 0.01-0.06 | NR | NR |
| 15F | CEO | 82-87 | 13-18 | F-AS | 0.01-0.06 | NR | NR |
| 15G | Comp | 83-86 | 14-17 | F-AS | 0.01-0.06 | NR | NR |
| 15H | CEO | 84-86 | 14-16 | F-AS | 0.01-0.06 | NR | NR |
| 15I | Comp | 80-<90 | >5-20 | F-AS | 0.01-0.06 | NR | NR |
| 15J | CEO | 80-<89 | >11-20 | F-AS | 0.01-0.06 | NR | NR |
| 15K | Comp | 80-88 | 12-20 | F-AS | 0.01-0.06 | NR | NR |
| 15L | CEO | 80-87 | 13-20 | F-AS | 0.01-0.06 | NR | NR |
| 15M | CEO | 80-86 | 14-20 | F-AS | 0.01-0.06 | NR | NR |
| 15N | Comp | 82-87 | 13-18 | F-AS | 0.01-0.06 | NR | NR |
| 15O | CEO | 83-86 | 14-17 | F-AS | 0.01-0.06 | NR | NR |
| 15P | Comp | 84-86 | 14-16 | F-AS | 0.01-0.06 | NR | NR |
| 15Q | CEO | 82-85 | 15-18 | F-AS | 0.01-0.06 | NR | NR |
| 15R | Comp | 83-85 | 15-17 | F-AS | 0.01-0.06 | NR | NR |
| 15S | CEO | 84 | 16 | F-AS | 0.01-0.06 | NR | NR |
| 15T | CEO | 85 | 15 | F-AS | 0.01-0.06 | NR | NR |
| 15U | CEO | 86 | 14 | F-AS | 0.01-0.06 | NR | NR |
| 16A | Comp | 80-<90 | >5-20 | F-AS | 0.03-0.04 | NR | NR |
| 16B | CEO | 80-<89 | >11-20 | F-AS | 0.03-0.04 | NR | NR |
| 16C | Comp | 80-88 | 12-20 | F-AS | 0.03-0.04 | NR | NR |
| 16D | CEO | 80-87 | 13-20 | F-AS | 0.03-0.04 | NR | NR |
| 16E | Comp | 80-86 | 14-20 | F-AS | 0.03-0.04 | NR | NR |
| 16F | CEO | 82-87 | 13-18 | F-AS | 0.03-0.04 | NR | NR |
| 16G | Comp | 83-86 | 14-17 | F-AS | 0.03-0.04 | NR | NR |
| 16H | CEO | 84-86 | 14-16 | F-AS | 0.03-0.04 | NR | NR |
| 16I | Comp | 80-<90 | >5-20 | F-AS | 0.03-0.04 | NR | NR |
| 16J | CEO | 80-<89 | >11-20 | F-AS | 0.03-0.04 | NR | NR |
| 16K | Comp | 80-88 | 12-20 | F-AS | 0.03-0.04 | NR | NR |
| 16L | CEO | 80-87 | 13-20 | F-AS | 0.03-0.04 | NR | NR |
| 16M | CEO | 80-86 | 14-20 | F-AS | 0.03-0.04 | NR | NR |
| 16N | Comp | 82-87 | 13-18 | F-AS | 0.03-0.04 | NR | NR |
| 16O | CEO | 83-86 | 14-17 | F-AS | 0.03-0.04 | NR | NR |
| 16P | Comp | 84-86 | 14-16 | F-AS | 0.03-0.04 | NR | NR |
| 16Q | CEO | 82-85 | 15-18 | F-AS | 0.03-0.04 | NR | NR |
| 16R | Comp | 83-85 | 15-17 | F-AS | 0.03-0.04 | NR | NR |
| 16S | CEO | 84 | 16 | F-AS | 0.03-0.04 | NR | NR |
| 16T | CEO | 85 | 15 | F-AS | 0.03-0.04 | NR | NR |
| 16U | CEO | 86 | 14 | F-AS | 0.03-0.04 | NR | NR |
| 17A | Comp | 80-<90 | >5-20 | FF | 0.03-0.04 | HCl | NR |
| 17B | CEO | 80-<89 | >11-20 | FF | 0.03-0.04 | HCl | NR |
| 17C | Comp | 80-88 | 12-20 | FF | 0.03-0.04 | HCl | NR |
| 17D | CEO | 80-87 | 13-20 | FF | 0.03-0.04 | HCl | NR |
| 17E | Comp | 80-86 | 14-20 | FF | 0.03-0.04 | HCl | NR |
| 17F | CEO | 82-87 | 13-18 | FF | 0.03-0.04 | HCl | NR |
| 17G | Comp | 83-86 | 14-17 | FF | 0.03-0.04 | HCl | NR |
| 17H | CEO | 84-86 | 14-16 | FF | 0.03-0.04 | HCl | NR |
| 17I | Comp | 80-<90 | >5-20 | FF | 0.03-0.04 | HCl | NR |
| 17J | CEO | 80-<89 | >11-20 | FF | 0.03-0.04 | HCl | NR |
| 17K | Comp | 80-88 | 12-20 | FF | 0.03-0.04 | HCl | NR |
| 17L | CEO | 80-87 | 13-20 | FF | 0.03-0.04 | HCl | NR |
| 17M | CEO | 80-86 | 14-20 | FF | 0.03-0.04 | HCl | NR |
| 17N | Comp | 82-87 | 13-18 | FF | 0.03-0.04 | HCl | NR |
| 17O | CEO | 83-86 | 14-17 | FF | 0.03-0.04 | HCl | NR |
| 17P | Comp | 84-86 | 14-16 | FF | 0.03-0.04 | HCl | NR |
| 17Q | CEO | 82-85 | 15-18 | FF | 0.03-0.04 | HCl | NR |
| 17R | Comp | 83-85 | 15-17 | FF | 0.03-0.04 | HCl | NR |
| 17S | CEO | 84 | 16 | FF | 0.03-0.04 | HCl | =<0.7 |
| 17T | CEO | 85 | 15 | FF | 0.03-0.04 | HCl | =<0.7 |
| 17U | CEO | 86 | 14 | FF | 0.03-0.04 | HCl | =<0.7 |
| 18A | Comp | 80-<90 | >5-20 | FF | 0.01-0.06 | NR | NR |
| 18B | CEO | 80-<89 | >11-20 | FF | 0.01-0.06 | NR | NR |
| 18C | Comp | 80-88 | 12-20 | FF | 0.01-0.06 | NR | NR |
| 18D | CEO | 80-87 | 13-20 | FF | 0.01-0.06 | NR | NR |
| 18E | Comp | 80-86 | 14-20 | FF | 0.01-0.06 | NR | NR |
| 18F | CEO | 82-87 | 13-18 | FF | 0.01-0.06 | NR | NR |
| 18G | Comp | 83-86 | 14-17 | FF | 0.01-0.06 | NR | NR |
| 18H | CEO | 84-86 | 14-16 | FF | 0.01-0.06 | NR | NR |
| 18I | Comp | 80-<90 | >5-20 | FF | 0.01-0.06 | NR | NR |
| 18J | CEO | 80-<89 | >11-20 | FF | 0.01-0.06 | NR | NR |
| 18K | Comp | 80-88 | 12-20 | FF | 0.01-0.06 | NR | NR |
| 18L | CEO | 80-87 | 13-20 | FF | 0.01-0.06 | NR | NR |
| 18M | CEO | 80-86 | 14-20 | FF | 0.01-0.06 | NR | NR |
| 18N | Comp | 82-87 | 13-18 | FF | 0.01-0.06 | NR | NR |
| 18O | CEO | 83-86 | 14-17 | FF | 0.01-0.06 | NR | NR |
| 18P | Comp | 84-86 | 14-16 | FF | 0.01-0.06 | NR | NR |
| 18Q | CEO | 82-85 | 15-18 | FF | 0.01-0.06 | NR | NR |
| 18R | Comp | 83-85 | 15-17 | FF | 0.01-0.06 | NR | NR |
| 18S | CEO | 84 | 16 | FF | 0.01-0.06 | NR | NR |
| 18T | CEO | 85 | 15 | FF | 0.01-0.06 | NR | NR |
| 18U | CEO | 86 | 14 | FF | 0.01-0.06 | NR | NR |
| 19A | Comp | 80-<90 | >5-20 | FF | 0.03-0.04 | NR | NR |
| 19B | CEO | 80-<89 | >11-20 | FF | 0.03-0.04 | NR | NR |
| 19C | Comp | 80-88 | 12-20 | FF | 0.03-0.04 | NR | NR |
| 19D | CEO | 80-87 | 13-20 | FF | 0.03-0.04 | NR | NR |
| 19E | Comp | 80-86 | 14-20 | FF | 0.03-0.04 | NR | NR |
| 19F | CEO | 82-87 | 13-18 | FF | 0.03-0.04 | NR | NR |
| 19G | Comp | 83-86 | 14-17 | FF | 0.03-0.04 | NR | NR |
| 19H | CEO | 84-86 | 14-16 | FF | 0.03-0.04 | NR | NR |
| 19I | Comp | 80-<90 | >5-20 | FF | 0.03-0.04 | NR | NR |
| 19J | CEO | 80-<89 | >11-20 | FF | 0.03-0.04 | NR | NR |
| 19K | Comp | 80-88 | 12-20 | FF | 0.03-0.04 | NR | NR |
| 19L | CEO | 80-87 | 13-20 | FF | 0.03-0.04 | NR | NR |
| 19M | CEO | 80-86 | 14-20 | FF | 0.03-0.04 | NR | NR |
| 19N | Comp | 82-87 | 13-18 | FF | 0.03-0.04 | NR | NR |
| 19O | CEO | 83-86 | 14-17 | FF | 0.03-0.04 | NR | NR |
| 19P | Comp | 84-86 | 14-16 | FF | 0.03-0.04 | NR | NR |
| 19Q | CEO | 82-85 | 15-18 | FF | 0.03-0.04 | NR | NR |
| 19R | Comp | 83-85 | 15-17 | FF | 0.03-0.04 | NR | NR |
| 19S | CEO | 84 | 16 | FF | 0.03-0.04 | NR | NR |
| 19T | CEO | 85 | 15 | FF | 0.03-0.04 | NR | NR |
| 19U | CEO | 86 | 14 | FF | 0.03-0.04 | NR | NR |
| 20A | Comp | 80-<90 | >5-20 | FF | 0.03-0.04 | HCl | NR |
| 20B | CEO | 80-<89 | >11-20 | FF | 0.03-0.04 | HCl | NR |
| 20C | Comp | 80-88 | 12-20 | FF | 0.03-0.04 | HCl | NR |
| 20D | CEO | 80-87 | 13-20 | FF | 0.03-0.04 | HCl | NR |
| 20E | Comp | 80-86 | 14-20 | FF | 0.03-0.04 | HCl | NR |
| 20F | CEO | 82-87 | 13-18 | FF | 0.03-0.04 | HCl | NR |
| 20G | Comp | 83-86 | 14-17 | FF | 0.03-0.04 | HCl | NR |
| 20H | CEO | 84-86 | 14-16 | FF | 0.03-0.04 | HCl | NR |
| 20I | Comp | 80-< | >5-20 | FF | 0.03-0.04 | HCl | NR |
| 20J | CEO | 80-<89 | >11-20 | FF | 0.03-0.04 | HCl | NR |
| 20K | Comp | 80-88 | 12-20 | FF | 0.03-0.04 | HCl | NR |
| 20L | CEO | 80-87 | 13-20 | FF | 0.03-0.04 | HCl | NR |
| 20M | CEO | 80-86 | 14-20 | FF | 0.03-0.04 | HCl | NR |
| 20N | Comp | 82-87 | 13-18 | FF | 0.03-0.04 | HCl | NR |
| 20O | CEO | 83-86 | 14-17 | FF | 0.03-0.04 | HCl | NR |
| 20P | Comp | 84-86 | 14-16 | FF | 0.03-0.04 | HCl | NR |
| 20Q | CEO | 82-85 | 15-18 | FF | 0.03-0.04 | HCl | NR |
| 20R | Comp | 83-85 | 15-17 | FF | 0.03-0.04 | HCl | NR |
| 20S | CEO | 84 | 16 | FF | 0.03-0.04 | HCl | =<0.7 |
| 20T | CEO | 85 | 15 | FF | 0.03-0.04 | HCl | =<0.7 |
| 20U | CEO | 86 | 14 | FF | 0.03-0.04 | HCl | =<0.7 |
| 21A | Comp | 80-<90 | >5-20 | G-AS | 0.01-0.06 | NR | NR |
| 21B | CEO | 80-<89 | >11-20 | G-AS | 0.01-0.06 | NR | NR |
| 21C | Comp | 80-88 | 12-20 | G-AS | 0.01-0.06 | NR | NR |
| 21D | CEO | 80-87 | 13-20 | G-AS | 0.01-0.06 | NR | NR |
| 21E | Comp | 80-86 | 14-20 | G-AS | 0.01-0.06 | NR | NR |
| 21F | CEO | 82-87 | 13-18 | G-AS | 0.01-0.06 | NR | NR |
| 21G | Comp | 83-86 | 14-17 | G-AS | 0.01-0.06 | NR | NR |
| 21H | CEO | 84-86 | 14-16 | G-AS | 0.01-0.06 | NR | NR |
| 21I | Comp | 80-<90 | >5-20 | G-AS | 0.01-0.06 | NR | NR |
| 21J | CEO | 80-<89 | >11-20 | G-AS | 0.01-0.06 | NR | NR |
| 21K | Comp | 80-88 | 12-20 | G-AS | 0.01-0.06 | NR | NR |
| 21L | CEO | 80-87 | 13-20 | G-AS | 0.01-0.06 | NR | NR |
| 21M | CEO | 80-86 | 14-20 | G-AS | 0.01-0.06 | NR | NR |
| 21N | Comp | 82-87 | 13-18 | G-AS | 0.01-0.06 | NR | NR |
| 21O | CEO | 83-86 | 14-17 | G-AS | 0.01-0.06 | NR | NR |
| 21P | Comp | 84-86 | 14-16 | G-AS | 0.01-0.06 | NR | NR |
| 21Q | CEO | 82-85 | 15-18 | G-AS | 0.01-0.06 | NR | NR |
| 21R | Comp | 83-85 | 15-17 | G-AS | 0.01-0.06 | NR | NR |
| 21S | CEO | 84 | 16 | G-AS | 0.01-0.06 | NR | NR |
| 21T | CEO | 85 | 15 | G-AS | 0.01-0.06 | NR | NR |
| 21U | CEO | 86 | 14 | G-AS | 0.01-0.06 | NR | NR |
| 22A | Comp | 80-<90 | >5-20 | G-AS | 0.03-0.04 | NR | NR |
| 22B | CEO | 80-<89 | >11-20 | G-AS | 0.03-0.04 | NR | NR |
| 22C | Comp | 80-88 | 12-20 | G-AS | 0.03-0.04 | NR | NR |
| 22D | CEO | 80-87 | 13-20 | G-AS | 0.03-0.04 | NR | NR |
| 22E | Comp | 80-86 | 14-20 | G-AS | 0.03-0.04 | NR | NR |
| 22F | CEO | 82-87 | 13-18 | G-AS | 0.03-0.04 | NR | NR |
| 22G | Comp | 83-86 | 14-17 | G-AS | 0.03-0.04 | NR | NR |
| 22H | CEO | 84-86 | 14-16 | G-AS | 0.03-0.04 | NR | NR |
| 22I | Comp | 80-<90 | >5-20 | G-AS | 0.03-0.04 | NR | NR |
| 22J | CEO | 80-<89 | >11-20 | G-AS | 0.03-0.04 | NR | NR |
| 22K | Comp | 80-88 | 12-20 | G-AS | 0.03-0.04 | NR | NR |
| 22L | CEO | 80-87 | 13-20 | G-AS | 0.03-0.04 | NR | NR |
| 22M | CEO | 80-86 | 14-20 | G-AS | 0.03-0.04 | NR | NR |
| 22N | Comp | 82-87 | 13-18 | G-AS | 0.03-0.04 | NR | NR |
| 22O | CEO | 83-86 | 14-17 | G-AS | 0.03-0.04 | NR | NR |
| 22P | Comp | 84-86 | 14-16 | G-AS | 0.03-0.04 | NR | NR |
| 22Q | CEO | 82-85 | 15-18 | G-AS | 0.03-0.04 | NR | NR |
| 22R | Comp | 83-85 | 15-17 | G-AS | 0.03-0.04 | NR | NR |
| 22S | CEO | 84 | 16 | G-AS | 0.03-0.04 | NR | NR |
| 22T | CEO | 85 | 15 | G-AS | 0.03-0.04 | NR | NR |
| 22U | CEO | 86 | 14 | G-AS | 0.03-0.04 | NR | NR |
| 23A | Comp | 80-<90 | >5-20 | GB | 0.03-0.04 | HCl | NR |
| 23B | CEO | 80-<89 | >11-20 | GB | 0.03-0.04 | HCl | NR |
| 23C | Comp | 80-88 | 12-20 | GB | 0.03-0.04 | HCl | NR |
| 23D | CEO | 80-87 | 13-20 | GB | 0.03-0.04 | HCl | NR |
| 23E | Comp | 80-86 | 14-20 | GB | 0.03-0.04 | HCl | NR |
| 23F | CEO | 82-87 | 13-18 | GB | 0.03-0.04 | HCl | NR |
| 23G | Comp | 83-86 | 14-17 | GB | 0.03-0.04 | HCl | NR |
| 23H | CEO | 84-86 | 14-16 | GB | 0.03-0.04 | HCl | NR |
| 23I | Comp | 80-<90 | >5-20 | GB | 0.03-0.04 | HCl | NR |
| 23J | CEO | 80-<89 | >11-20 | GB | 0.03-0.04 | HCl | NR |
| 23K | Comp | 80-88 | 12-20 | GB | 0.03-0.04 | HCl | NR |
| 23L | CEO | 80-87 | 13-20 | GB | 0.03-0.04 | HCl | NR |
| 23M | CEO | 80-86 | 14-20 | GB | 0.03-0.04 | HCl | NR |
| 23N | Comp | 82-87 | 13-18 | GB | 0.03-0.04 | HCl | NR |
| 23O | CEO | 83-86 | 14-17 | GB | 0.03-0.04 | HCl | NR |
| 23P | Comp | 84-86 | 14-16 | GB | 0.03-0.04 | HCl | NR |
| 23Q | CEO | 82-85 | 15-18 | GB | 0.03-0.04 | HCl | NR |
| 23R | Comp | 83-85 | 15-17 | GB | 0.03-0.04 | HCl | NR |
| 23S | CEO | 84 | 16 | GB | 0.03-0.04 | HCl | =<0.7 |
| 23T | CEO | 85 | 15 | GB | 0.03-0.04 | HCl | =<0.7 |
| 23U | CEO | 86 | 14 | GB | 0.03-0.04 | HCl | =<0.7 |
| 24A | Comp | 80-<90 | >5-20 | GB | 0.01-0.06 | NR | NR |
| 24B | CEO | 80-<89 | >11-20 | GB | 0.01-0.06 | NR | NR |
| 24C | Comp | 80-88 | 12-20 | GB | 0.01-0.06 | NR | NR |
| 24D | CEO | 80-87 | 13-20 | GB | 0.01-0.06 | NR | NR |
| 24E | Comp | 80-86 | 14-20 | GB | 0.01-0.06 | NR | NR |
| 24F | CEO | 82-87 | 13-18 | GB | 0.01-0.06 | NR | NR |
| 24G | Comp | 83-86 | 14-17 | GB | 0.01-0.06 | NR | NR |
| 24H | CEO | 84-86 | 14-16 | GB | 0.01-0.06 | NR | NR |
| 24I | Comp | 80-<90 | >5-20 | GB | 0.01-0.06 | NR | NR |
| 24J | CEO | 80-<89 | >11-20 | GB | 0.01-0.06 | NR | NR |
| 24K | Comp | 80-88 | 12-20 | GB | 0.01-0.06 | NR | NR |
| 24L | CEO | 80-87 | 13-20 | GB | 0.01-0.06 | NR | NR |
| 24M | CEO | 80-86 | 14-20 | GB | 0.01-0.06 | NR | NR |
| 24N | Comp | 82-87 | 13-18 | GB | 0.01-0.06 | NR | NR |
| 24O | CEO | 83-86 | 14-17 | GB | 0.01-0.06 | NR | NR |
| 24P | Comp | 84-86 | 14-16 | GB | 0.01-0.06 | NR | NR |
| 24Q | CEO | 82-85 | 15-18 | GB | 0.01-0.06 | NR | NR |
| 24R | Comp | 83-85 | 15-17 | GB | 0.01-0.06 | NR | NR |
| 24S | CEO | 84 | 16 | GB | 0.01-0.06 | NR | NR |
| 24T | CEO | 85 | 15 | GB | 0.01-0.06 | NR | NR |
| 24U | CEO | 86 | 14 | GB | 0.01-0.06 | NR | NR |
| 25A | Comp | 80-<90 | >5-20 | GB | 0.03-0.04 | NR | NR |
| 25B | CEO | 80-<89 | >11-20 | GB | 0.03-0.04 | NR | NR |
| 25C | Comp | 80-88 | 12-20 | GB | 0.03-0.04 | NR | NR |
| 25D | CEO | 80-87 | 13-20 | GB | 0.03-0.04 | NR | NR |
| 25E | Comp | 80-86 | 14-20 | GB | 0.03-0.04 | NR | NR |
| 25F | CEO | 82-87 | 13-18 | GB | 0.03-0.04 | NR | NR |
| 25G | Comp | 83-86 | 14-17 | GB | 0.03-0.04 | NR | NR |
| 25H | CEO | 84-86 | 14-16 | GB | 0.03-0.04 | NR | NR |
| 25I | Comp | 80-<90 | >5-20 | GB | 0.03-0.04 | NR | NR |
| 25J | CEO | 80-<89 | >11-20 | GB | 0.03-0.04 | NR | NR |
| 25K | Comp | 80-88 | 12-20 | GB | 0.03-0.04 | NR | NR |
| 25L | CEO | 80-87 | 13-20 | GB | 0.03-0.04 | NR | NR |
| 25M | CEO | 80-86 | 14-20 | GB | 0.03-0.04 | NR | NR |
| 25N | Comp | 82-87 | 13-18 | GB | 0.03-0.04 | NR | NR |
| 25O | CEO | 83-86 | 14-17 | GB | 0.03-0.04 | NR | NR |
| 25P | Comp | 84-86 | 14-16 | GB | 0.03-0.04 | NR | NR |
| 25Q | CEO | 82-85 | 15-18 | GB | 0.03-0.04 | NR | NR |
| 25R | Comp | 83-85 | 15-17 | GB | 0.03-0.04 | NR | NR |
| 25S | CEO | 84 | 16 | GB | 0.03-0.04 | NR | NR |
| 25T | CEO | 85 | 15 | GB | 0.03-0.04 | NR | NR |
| 25U | CEO | 86 | 14 | GB | 0.03-0.04 | NR | NR |
| 26A | Comp | 80-<90 | >5-20 | GB | 0.03-0.04 | HCl | NR |
| 26B | CEO | 80-<89 | >11-20 | GB | 0.03-0.04 | HCl | NR |
| 26C | Comp | 80-88 | 12-20 | GB | 0.03-0.04 | HCl | NR |
| 26D | CEO | 80-87 | 13-20 | GB | 0.03-0.04 | HCl | NR |
| 26E | Comp | 80-86 | 14-20 | GB | 0.03-0.04 | HCl | NR |
| 26F | CEO | 82-87 | 13-18 | GB | 0.03-0.04 | HCl | NR |
| 26G | Comp | 83-86 | 14-17 | GB | 0.03-0.04 | HCl | NR |
| 26H | CEO | 84-86 | 14-16 | GB | 0.03-0.04 | HCl | NR |
| 26I | Comp | 80-<90 | >5-20 | GB | 0.03-0.04 | HCl | NR |
| 26J | CEO | 80-<89 | >11-20 | GB | 0.03-0.04 | HCl | NR |
| 26K | Comp | 80-88 | 12-20 | GB | 0.03-0.04 | HCl | NR |
| 26L | CEO | 80-87 | 13-20 | GB | 0.03-0.04 | HCl | NR |
| 26M | CEO | 80-86 | 14-20 | GB | 0.03-0.04 | HCl | NR |
| 26N | Comp | 82-87 | 13-18 | GB | 0.03-0.04 | HCl | NR |
| 26O | CEO | 83-86 | 14-17 | GB | 0.03-0.04 | HCl | NR |
| 26P | Comp | 84-86 | 14-16 | GB | 0.03-0.04 | HCl | NR |
| 26Q | CEO | 82-85 | 15-18 | GB | 0.03-0.04 | HCl | NR |
| 26R | Comp | 83-85 | 15-17 | GB | 0.03-0.04 | HCl | NR |
| 26S | CEO | 84 | 16 | GB | 0.03-0.04 | HCl | =<0.7 |
| 26T | CEO | 85 | 15 | GB | 0.03-0.04 | HCl | =<0.7 |
| 26U | CEO | 86 | 14 | GB | 0.03-0.04 | HCl | =<0.7 |

The present invention includes pharmaceutical compositions, including each of Pharmaceutical Compositions 1-5 and PCs 1-26, and each pharmaceutical composition used in each of Pharmaceutical Methods 1-6 and in MDI1, in which the composition is substantially free of API which is not in solution. In other words, the present invention includes pharmaceutical compositions, including each of Pharmaceutical Compositions 1-5 and PCs 1-26, and each pharmaceutical composition used in each of Pharmaceutical Methods 1-6 and in MDI1, in which essentially all of the recited API is in solution in said 1234ze(E) and/or said ethanol.

### III. Devices and Methods

The present invention includes devices for the delivery by inhalation of the compositions and formulations of the present invention, including each of MDI1 through MDI2. In certain preferred embodiments, the devices of the present invention, including each of MDI1 through MDI2, comprise a container, preferably an aerosol canister, containing a pressurized composition of the present invention and having a metered dose dispensing valve operable between non-dispensing and dispensing positions. The present devices, including each of MDI1 through MDI2, preferably also comprise an actuator, which in preferred embodiments comprises a housing adapted to receive the aerosol container and to define a chamber in fluid communication with a patient port for introducing the medicament into the oral and/or nasal cavity of the patient, preferably in the form of a mouthpiece and/or nasal adapter. The actuator also preferably includes a nozzle block adapted to receive the valve stem of the dispensing valve, the nozzle block preferably comprising a passage in fluid communication with the valve stem and terminating in an orifice for directing medicament from the valve stem into the chamber.

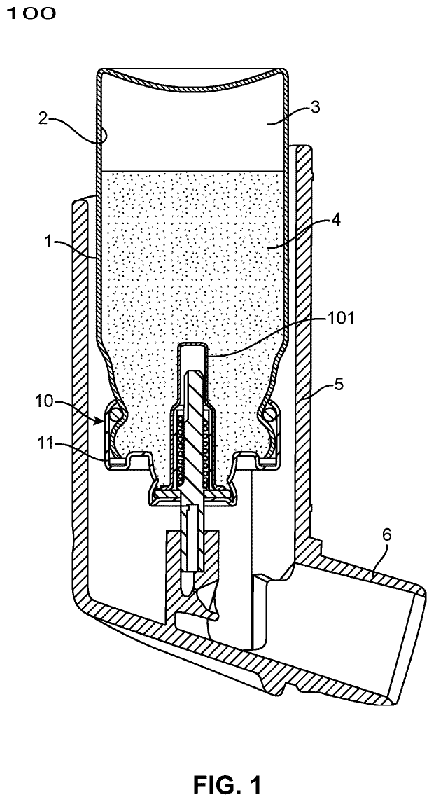

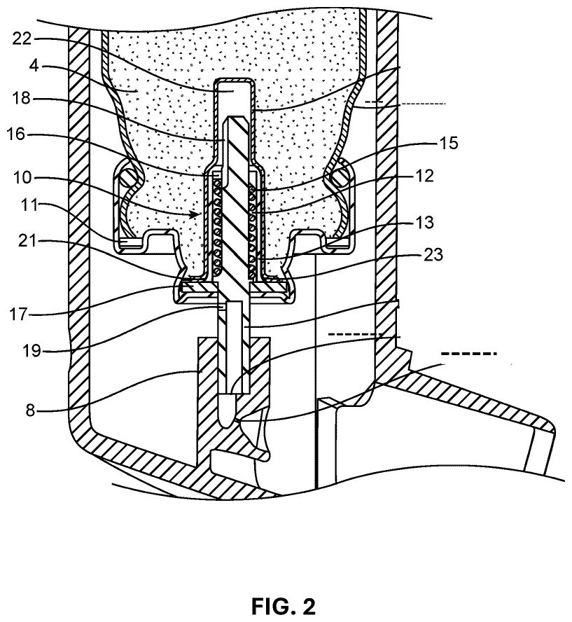

By way of example but not by way of limitation, FIG. 1 shows one embodiment of a metered dose inhaler 100, including an aerosol canister 1 fitted with a metered dose metering valve 10 (shown in its resting position). The metering valve 10 is typically affixed, i.e., crimped, onto the canister via a cap or ferrule 11 (typically made of aluminum or an aluminum alloy) which is generally provided as part of the valve assembly. Between the canister and the ferrule there may be one or more seals. In the embodiments shown in FIG. 1 and FIG. 2 between the canister 1 and the ferrule 11 there are two seals including, e.g., an O-ring seal and a gasket seal.

As shown in FIG. 1, the canister/valve dispenser is typically provided with an actuator 5 including an appropriate patient port 6, such as a mouthpiece. For administration to the nasal cavities the patient port is generally provided in an appropriate form (e.g., smaller diameter tube, often sloping upwardly) for delivery through the nose. Actuators are generally made of a plastic material, for example polypropylene or polyethylene. As can be seen from FIG. 1, inner walls 2 of the canister and outer walls 101 of the portion(s) of the metering valve 10 located within the canister define a formulation chamber 3 in which aerosol composition 4 is contained.

The valve 10 shown in FIG. 1 and FIG. 2, includes a metering chamber 12, defined in part by an inner valve body 13, through which a valve stem 14 passes. The valve stem 14, which is biased outwardly by a compression spring 15, is in sliding sealing engagement with an inner tank seal 16 and an outer diaphragm seal 17. The valve 10 also includes a second valve body 20 in the form of a bottle emptier. The inner valve body 13 (also referred to as the “primary” valve body) defines in part the metering chamber 12. The second valve body 20 (also referred to as the “secondary” valve body) defines in part a pre-metering region or chamber besides serving as a bottle emptier.

Referring to FIG. 2, aerosol composition 4 can pass from the composition chamber 3 into a pre-metering chamber 22 provided between the secondary valve body 20 and the primary valve body 13 through an annular space 21 between a flange 23 of the secondary valve body 20 and the primary valve body 13. To actuate (fire) the valve 10, the valve stem 14 is pushed inwardly relative to the canister 1 from its resting position shown in FIG. 1 and FIG. 2, allowing composition to pass from the metering chamber 12 through a side hole 19 in the valve stem and through a stem outlet 24 to a nozzle block 8, which includes an actuator nozzle comprising an orifice 7 then out to the patient through port 6 (see FIG. 1). When the valve stem 14 is released, composition enters into the valve 10, in particular into the pre-metering chamber 22, through the annular space 21 and thence from the pre-metering chamber through a groove 18 in the valve stem past the tank seal 16 into the metering chamber 12.

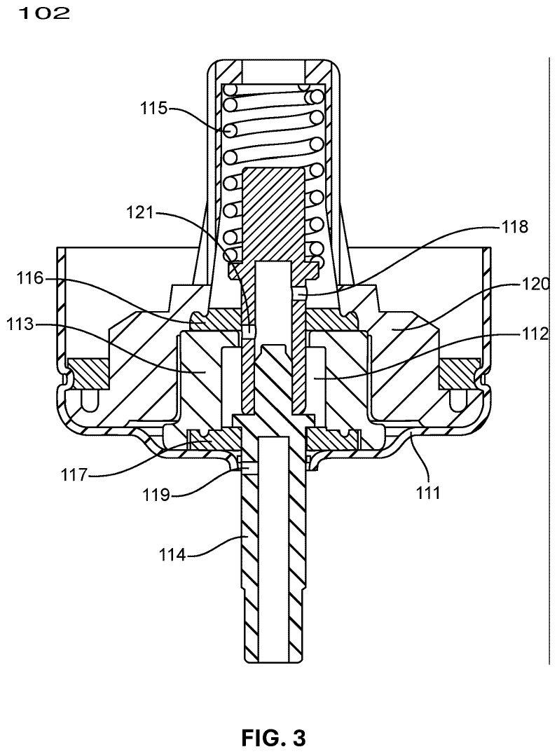

FIG. 3 shows another embodiment of a metered dose aerosol metering valve 102, different from the embodiment shown in FIG. 1 and FIG. 2, in its rest position. The valve 102 has a metering chamber 112 defined in part by a metering tank 113 through which a stem 114 is biased outwardly by spring 115. The stem 114 is made in two parts that are push fit together before being assembled into valve 102. The stem 114 has an inner seal 116 and an outer seal 117 disposed about it and forming sealing contact with the metering tank 113. A valve body 120 crimped into a ferrule 111 retains the aforementioned components in the valve. In use, composition enters the metering chamber via orifices 121 and 118. The composition's outward path from the metering chamber 112 when a dose is dispensed is via orifice 119.

In certain embodiments the invention, including each of MDI1 through MDI2, is constructed such that airflow due to patient inhalation is prevented or reduced in the vicinity of the orifice at all times or except during dispensing of the medicament from the valve. Either of such arrangements has the effect of substantially reducing the velocity of the emitted spray compared to an inhaler which allows free flow of air in the vicinity of the nozzle block during dispensing of the medicament.

In certain embodiments, the actuator is constructed such that the distance from the nozzle to the mouthpiece is from approximately 1 to 15 cm, preferably 4 to 6 cm, with a chamber/mouthpiece diameter from 1 to 4 cm, 0.5 to 1 cm in the case of a nasal adapter.

In certain preferred but non-limiting embodiments, the actuator possesses air inlets which enable the patient to inhale though the patient port, preferably without encountering significant resistance since the patient may have breathing difficulties when taking the medication, for example, during an asthma attack. However, the air inlets, for example in the mouthpiece, preferably do not concentrate the airflow into an area that is too narrow, as this will give a high velocity of incoming air which will deflect the spray onto the wall of the mouthpiece opposite the air inlets. In certain preferred embodiments the air inlets are positioned downstream of the nozzle, in the region of the turbulent zone and/or downstream of the turbulent zone. The positioning and direction of the air inlets may also affect the deposition of medicament within the chamber and mouthpiece. In one arrangement air inlets comprise a series of holes and optionally may be interspersed with fluid deflection structures on the wall of the chamber, to direct air into the turbulent zone to mix air with the aerosol stream. Further, the mouthpiece may be constructed of porous material to allow a multiplicity of finely divided air vents to provide air flow over a larger surface area.

In certain embodiments the actuator possesses air inlets upstream of or in the vicinity of the nozzle, but the air inlets are blocked when the valve is fired to release the aerosol spray. The air inlets are opened after the spray has been released by which time the velocity of the stream will have been reduced and the turbulent zone formed. Upon inhalation, an airflow is established from the air inlets to the mouthpiece which entrains the residual aerosol spray. The actuator may include additional air inlets downstream of the nozzle, as described above with respect to the first embodiment. These downstream air inlets do not need to close during release of the aerosol spray.

In certain embodiments, a porous membrane is present to introduce air into or downstream of the turbulent zone. One advantage of the use of such a membrane is that the air is introduced more uniformly and diffusely around the circumference of the spray, thereby acting as a buffer between the turbulent flow and the wall. The effect is to reduce drug deposition in the device. The membrane may optionally be protected from dirt or contact by the user's lips by an additional part of the mouthpiece. When present, it is preferred that the porous membrane material (50) must not significantly impede the patient's ability to inhale through the device. A suitable material is Whatmann No. 4 filter paper; but other materials may be used, such as those used in cylindrical air filters or membrane filters, or such as those formed by sintering polymers. A preferred porous membrane material is in the form of a cylinder made by fusing together small pellets of polypropylene.

For certain medicaments, it is preferred to configure the device so as to reduce contact between the medicament and parts of the patient's body that it is not intended to contact. For example, residues of the medicament deposited on internal surfaces of actuators may be fingered and transferred to other body parts. In such cases, the device may be configured to include one or more fluid flow deflectors to allow the spray to pass through, whilst limiting access by the patient to internal surfaces of the actuator. Of course, the device may be configured for intranasal delivery. This is normally quite undesirable, since the medicaments were designed for delivery to the respiratory system and may not have an appropriate effect when deposited in the oropharynx and allowed to enter the digestive tract. In an effort to overcome this problem, certain embodiments of the present device include the provision of a holding volume, commonly called a spacer, in which the medicament is fired. The spacer preferably allows the velocity of the medicament to be reduced and may also allow some propellant evaporation to occur. Spacers can improve the performance of a metered dose inhaler by reducing oropharyngeal deposition.

The total amount of composition or formulation of the present of the present invention, including each of PC1-PC26, contained in the canister of the present pMDIs, including each of MDI1 through MDI2, preferably is selected so that at least a portion of the propellant in the canister is present as a liquid after a predetermined number of medicinal doses have been delivered. The predetermined number of doses may be 5 to 200, 30 to 200, 60 to 200, 60 to 120, 60, 120, 200, or any other number of doses. In preferred embodiments, the total amount of composition of the present invention in the canister may be from 1.0 grams (g) to 30.0 g, 2.0 g to 20.0 g, or 5.0 to 10.0 g. The total amount of formulation, including each of PC1 through PC26 contained in the canister of present pMDIs, including each of MDI1 through MDI2, is preferably selected to be greater than the product of the predetermined number of doses and the metering volume of the metering valve. In some embodiments, the total amount of composition is greater than 1.1 times, greater than 1.2 times, greater than 1.3 times, greater than 1.4 times, or greater than 1.5 times the product of the predetermined number of doses and the metering volume of the metering valve. This helps to ensure that the amount of each dose remains relatively constant through the life of the inhaler.

The present pMDIs, including each of MDI1 through MDI2, comprises: a composition of the invention and an actuator orifice having a diameter of greater than 0.15 mm and less than 0.5 mm. Particular MDIs include those identified in the following Table pMDI, wherein: the first column of the table includes “pMDI” as an abbreviation for the pMDI defined in the table; the column headed Pharmaceutical Composition means a pharmaceutical compositions as defined in Table PC above contained in the canister of the pMDI; “NR” means that the component or a particular size “not required” according to the specified pMDI definition and as such its presence in any size is permitted; “Yes” means the component is required but that any type or size is permitted; “Comp” means that the specified pMDI comprises the items identified in the table; and all sizes are understood to be preceded by the word “about.”

TABLE HTC

| | | | | Actuation | |
|---|---|---|---|---|---|
| | | | | Property | |
| | | | Orifice | | DD % |
| pMDI, | | Pharmaceutical | Diameter, | | Rel. |
| No. | | Composition | mm | DD % | 134a |
| 3A | Comp | 1A | <0.5 | NR | NR |
| 3B | CEO | 1B | <0.5 | NR | NR |
| 3C | Comp | 1C | <0.5 | NR | NR |
| 3D | CEO | 1D | <0.5 | NR | NR |
| 3E | Comp | 1E | <0.5 | NR | NR |
| 3F | CEO | 1F | <0.5 | NR | NR |
| 3G | Comp | 1G | <0.5 | NR | NR |
| 3H | CEO | 1H | <0.5 | NR | NR |
| 3I | Comp | 1I | <0.5 | NR | NR |
| 3J | CEO | 1J | <0.5 | NR | NR |
| 3K | Comp | 1K | <0.5 | NR | NR |
| 3L | CEO | 1L | <0.5 | NR | NR |
| 3M | CEO | 1M | <0.5 | NR | NR |
| 3N | Comp | 1N | <0.5 | NR | NR |
| 3O | CEO | 1O | <0.5 | NR | NR |
| 3P | Comp | 1P | <0.5 | NR | NR |
| 3Q | CEO | 1Q | <0.5 | NR | NR |
| 3R | Comp | 1R | <0.5 | NR | NR |
| 3S | CEO | 1S | <0.5 | NR | NR |
| 3T | CEO | 1T | <0.5 | NR | NR |
| 3U | CEO | 1U | <0.5 | NR | NR |
| 4A | Comp | 2A | <0.5 | NR | NR |
| 4B | CEO | 2B | <0.5 | NR | NR |
| 4C | Comp | 2C | <0.5 | NR | NR |
| 4D | CEO | 2D | <0.5 | NR | NR |
| 4E | Comp | 2E | <0.5 | NR | NR |
| 4F | CEO | 2F | <0.5 | NR | NR |
| 4G | Comp | 2G | <0.5 | NR | NR |
| 4H | CEO | 2H | <0.5 | NR | NR |
| 4I | Comp | 2I | <0.5 | NR | NR |
| 4J | CEO | 2J | <0.5 | NR | NR |
| 4K | Comp | 2K | <0.5 | NR | NR |
| 4L | CEO | 2L | <0.5 | NR | NR |
| 4M | CEO | 2M | <0.5 | NR | NR |
| 4N | Comp | 2N | <0.5 | NR | NR |
| 4O | CEO | 2O | <0.5 | NR | NR |
| 4P | Comp | 2P | <0.5 | NR | NR |
| 4Q | CEO | 2Q | <0.5 | NR | NR |
| 4R | Comp | 2R | <0.5 | NR | NR |
| 4S | CEO | 2S | <0.5 | NR | NR |
| 4T | CEO | 2T | <0.5 | NR | NR |
| 4U | CEO | 2U | <0.5 | NR | NR |
| 5A | Comp | 3A | <0.5 | NR | NR |
| 5B | CEO | 3B | <0.5 | NR | NR |
| 5C | Comp | 3C | <0.5 | NR | NR |
| 5D | CEO | 3D | <0.5 | NR | NR |
| 5E | Comp | 3E | <0.5 | NR | NR |
| 5F | CEO | 3F | <0.5 | NR | NR |
| 5G | Comp | 3G | <0.5 | NR | NR |
| 5H | CEO | 3H | <0.5 | NR | NR |
| 5I | Comp | 3I | <0.5 | NR | NR |
| 5J | CEO | 3J | <0.5 | NR | NR |
| 5K | Comp | 3K | <0.5 | NR | NR |
| 5L | CEO | 3L | <0.5 | NR | NR |
| 5M | CEO | 3M | <0.5 | NR | NR |
| 5N | Comp | 3N | <0.5 | NR | NR |
| 5O | CEO | 3O | <0.5 | NR | NR |
| 5P | Comp | 3P | <0.5 | NR | NR |
| 5Q | CEO | 3Q | <0.5 | NR | NR |
| 5R | Comp | 3R | <0.5 | NR | NR |
| 5S | CEO | 3S | <0.5 | NR | NR |
| 5T | CEO | 3T | <0.5 | NR | NR |
| 5U | CEO | 3U | <0.5 | NR | NR |
| 6A | Comp | 4A | <0.5 | NR | NR |
| 6B | CEO | 4B | <0.5 | NR | NR |
| 6C | Comp | 4C | <0.5 | NR | NR |
| 6D | CEO | 4D | <0.5 | NR | NR |
| 6E | Comp | 4E | <0.5 | NR | NR |
| 6F | CEO | 4F | <0.5 | NR | NR |
| 6G | Comp | 4G | <0.5 | NR | NR |
| 6H | CEO | 4H | <0.5 | NR | NR |
| 6I | Comp | 4I | <0.5 | NR | NR |
| 6J | CEO | 4J | <0.5 | NR | NR |
| 6K | Comp | 4K | <0.5 | NR | NR |
| 6L | CEO | 4L | <0.5 | NR | NR |
| 6M | CEO | 4M | <0.5 | NR | NR |
| 6N | Comp | 4N | <0.5 | NR | NR |
| 6O | CEO | 4O | <0.5 | NR | NR |
| 6P | Comp | 4P | <0.5 | NR | NR |
| 6Q | CEO | 4Q | <0.5 | NR | NR |
| 6R | Comp | 4R | <0.5 | NR | NR |
| 6S | CEO | 4S | <0.5 | NR | NR |
| 6T | CEO | 4T | <0.5 | NR | NR |
| 6U | CEO | 4U | <0.5 | NR | NR |
| 7A | Comp | 5A | <0.5 | NR | NR |
| 7B | CEO | 5B | <0.5 | NR | NR |
| 7C | Comp | 5C | <0.5 | NR | NR |
| 7D | CEO | 5D | <0.5 | NR | NR |
| 7E | Comp | 5E | <0.5 | NR | NR |
| 7F | CEO | 5F | <0.5 | NR | NR |
| 7G | Comp | 5G | <0.5 | NR | NR |
| 7H | CEO | 5H | <0.5 | NR | NR |
| 7I | Comp | 5I | <0.5 | NR | NR |
| 7J | CEO | 5J | <0.5 | NR | NR |
| 7K | Comp | 5K | <0.5 | NR | NR |
| 7L | CEO | 5L | <0.5 | NR | NR |
| 7M | CEO | 5M | <0.5 | NR | NR |
| 7N | Comp | 5N | <0.5 | NR | NR |
| 7O | CEO | 5O | <0.5 | NR | NR |
| 7P | Comp | 5P | <0.5 | NR | NR |
| 7Q | CEO | 5Q | <0.5 | NR | NR |
| 7R | Comp | 5R | <0.5 | NR | NR |
| 7S | CEO | 5S | <0.5 | NR | NR |
| 7T | CEO | 5T | <0.5 | NR | NR |
| 7U | CEO | 5U | <0.5 | NR | NR |
| 8A | Comp | 6A | <0.5 | NR | NR |
| 8B | CEO | 6B | <0.5 | NR | NR |
| 8C | Comp | 6C | <0.5 | NR | NR |
| 8D | CEO | 6D | <0.5 | NR | NR |
| 8E | Comp | 6E | <0.5 | NR | NR |
| 8F | CEO | 6F | <0.5 | NR | NR |
| 8G | Comp | 6G | <0.5 | NR | NR |
| 8H | CEO | 6H | <0.5 | NR | NR |
| 8I | Comp | 6I | <0.5 | NR | NR |
| 8J | CEO | 6J | <0.5 | NR | NR |
| 8K | Comp | 6K | <0.5 | NR | NR |
| 8L | CEO | 6L | <0.5 | NR | NR |
| 8M | CEO | 6M | <0.5 | NR | NR |
| 8N | Comp | 6N | <0.5 | NR | NR |
| 8O | CEO | 6O | <0.5 | NR | NR |
| 8P | Comp | 6P | <0.5 | NR | NR |
| 8Q | CEO | 6Q | <0.5 | NR | NR |
| 8R | Comp | 6R | <0.5 | NR | NR |
| 8S | CEO | 6S | <0.5 | NR | NR |
| 8T | CEO | 6T | <0.5 | NR | NR |
| 8U | CEO | 6U | <0.5 | NR | NR |
| 9A | Comp | 7A | <0.5 | NR | NR |
| 9B | CEO | 7B | <0.5 | NR | NR |
| 9C | Comp | 7C | <0.5 | NR | NR |
| 9D | CEO | 7D | <0.5 | NR | NR |
| 9E | Comp | 7E | <0.5 | NR | NR |
| 9F | CEO | 7F | <0.5 | NR | NR |
| 9G | Comp | 7G | <0.5 | NR | NR |
| 9H | CEO | 7H | <0.5 | NR | NR |
| 9I | Comp | 7I | <0.5 | NR | NR |
| 9J | CEO | 7J | <0.5 | NR | NR |
| 9K | Comp | 7K | <0.5 | NR | NR |
| 9L | CEO | 7L | <0.5 | NR | NR |
| 9M | CEO | 7M | <0.5 | NR | NR |
| 9N | Comp | 7N | <0.5 | NR | NR |
| 9O | CEO | 7O | <0.5 | NR | NR |
| 9P | Comp | 7P | <0.5 | NR | NR |
| 9Q | CEO | 7Q | <0.5 | NR | NR |
| 9R | Comp | 7R | <0.5 | NR | NR |
| 9S | CEO | 7S | <0.5 | NR | NR |
| 9T | CEO | 7T | <0.5 | NR | NR |
| 9U | CEO | 7U | <0.5 | NR | NR |
| 10A | Comp | 8A | <0.5 | NR | NR |
| 10B | CEO | 8B | <0.5 | NR | NR |
| 10C | Comp | 8C | <0.5 | NR | NR |
| 10D | CEO | 8D | <0.5 | NR | NR |
| 10E | Comp | 8E | <0.5 | NR | NR |
| 10F | CEO | 8F | <0.5 | NR | NR |
| 10G | Comp | 8G | <0.5 | NR | NR |
| 10H | CEO | 8H | <0.5 | NR | NR |
| 10I | Comp | 8I | <0.5 | NR | NR |
| 10J | CEO | 8J | <0.5 | NR | NR |
| 10K | Comp | 8K | <0.5 | NR | NR |
| 10L | CEO | 8L | <0.5 | NR | NR |
| 10M | CEO | 8M | <0.5 | NR | NR |
| 10N | Comp | 8N | <0.5 | NR | NR |
| 10O | CEO | 8O | <0.5 | NR | NR |
| 10P | Comp | 8P | <0.5 | NR | NR |
| 10Q | CEO | 8Q | <0.5 | NR | NR |
| 10R | Comp | 8R | <0.5 | NR | NR |
| 10S | CEO | 8S | <0.5 | NR | NR |
| 10T | CEO | 8T | <0.5 | NR | NR |
| 10U | CEO | 8U | <0.5 | NR | NR |
| 11A | Comp | 9A | <0.5 | NR | NR |
| 11B | CEO | 9B | <0.5 | NR | NR |
| 11C | Comp | 9C | <0.5 | NR | NR |
| 11D | CEO | 9D | <0.5 | NR | NR |
| 11E | Comp | 9E | <0.5 | NR | NR |
| 11F | CEO | 9F | <0.5 | NR | NR |
| 11G | Comp | 9G | <0.5 | NR | NR |
| 11H | CEO | 9H | <0.5 | NR | NR |
| 11I | Comp | 9I | <0.5 | NR | NR |
| 11J | CEO | 9J | <0.5 | NR | NR |
| 11K | Comp | 9K | <0.5 | NR | NR |
| 11L | CEO | 9L | <0.5 | NR | NR |
| 11M | CEO | 9M | <0.5 | NR | NR |
| 11N | Comp | 9N | <0.5 | NR | NR |
| 11O | CEO | 9O | <0.5 | NR | NR |
| 11P | Comp | 9P | <0.5 | NR | NR |
| 11Q | CEO | 9Q | <0.5 | NR | NR |
| 11R | Comp | 9R | <0.5 | NR | NR |
| 11S | CEO | 9S | <0.5 | NR | NR |
| 11T | CEO | 9T | <0.5 | NR | NR |
| 11U | CEO | 9U | <0.5 | NR | NR |
| 12A | Comp | 10A | <0.5 | NR | NR |
| 12B | CEO | 10B | <0.5 | NR | NR |
| 12C | Comp | 10C | <0.5 | NR | NR |
| 12D | CEO | 10D | <0.5 | NR | NR |
| 12E | Comp | 10E | <0.5 | NR | NR |
| 12F | CEO | 10F | <0.5 | NR | NR |
| 12G | Comp | 10G | <0.5 | NR | NR |
| 12H | CEO | 10H | <0.5 | NR | NR |
| 12I | Comp | 10I | <0.5 | NR | NR |
| 12J | CEO | 10J | <0.5 | NR | NR |
| 12K | Comp | 10K | <0.5 | NR | NR |
| 12L | CEO | 10L | <0.5 | NR | NR |
| 12M | CEO | 10M | <0.5 | NR | NR |
| 12N | Comp | 10N | <0.5 | NR | NR |
| 12O | CEO | 10O | <0.5 | NR | NR |
| 12P | Comp | 10P | <0.5 | NR | NR |
| 12Q | CEO | 10Q | <0.5 | NR | NR |
| 12R | Comp | 10R | <0.5 | NR | NR |
| 12S | CEO | 10S | <0.5 | NR | NR |
| 12T | CEO | 10T | <0.5 | NR | NR |
| 12U | CEO | 10U | <0.5 | NR | NR |
| 13A | Comp | 11A | <0.5 | NR | NR |
| 13B | CEO | 11B | <0.5 | NR | NR |
| 13C | Comp | 11C | <0.5 | NR | NR |
| 13D | CEO | 11D | <0.5 | NR | NR |
| 13E | Comp | 11E | <0.5 | NR | NR |
| 13F | CEO | 11F | <0.5 | NR | NR |
| 13G | Comp | 11G | <0.5 | NR | NR |
| 13H | CEO | 11H | <0.5 | NR | NR |
| 13I | Comp | 11I | <0.5 | NR | NR |
| 13J | CEO | 11J | <0.5 | NR | NR |
| 13K | Comp | 11K | <0.5 | NR | NR |
| 13L | CEO | 11L | <0.5 | NR | NR |
| 13M | CEO | 11M | <0.5 | NR | NR |
| 13N | Comp | 11N | <0.5 | NR | NR |
| 13O | CEO | 11O | <0.5 | NR | NR |
| 13P | Comp | 11P | <0.5 | NR | NR |
| 13Q | CEO | 11Q | <0.5 | NR | NR |
| 13R | Comp | 11R | <0.5 | NR | NR |
| 13S | CEO | 11S | <0.5 | NR | NR |
| 13T | CEO | 11T | <0.5 | NR | NR |
| 13U | CEO | 11U | <0.5 | NR | NR |
| 14A | Comp | 12A | <0.5 | NR | NR |
| 14B | CEO | 12B | <0.5 | NR | NR |
| 14C | Comp | 12C | <0.5 | NR | NR |
| 14D | CEO | 12D | <0.5 | NR | NR |
| 14E | Comp | 12E | <0.5 | NR | NR |
| 14F | CEO | 12F | <0.5 | NR | NR |
| 14G | Comp | 12G | <0.5 | NR | NR |
| 14H | CEO | 12H | <0.5 | NR | NR |
| 14I | Comp | 12I | <0.5 | NR | NR |
| 14J | CEO | 12J | <0.5 | NR | NR |
| 14K | Comp | 12K | <0.5 | NR | NR |
| 14L | CEO | 12L | <0.5 | NR | NR |
| 14M | CEO | 12M | <0.5 | NR | NR |
| 14N | Comp | 12N | <0.5 | NR | NR |
| 14O | CEO | 12O | <0.5 | NR | NR |
| 14P | Comp | 12P | <0.5 | NR | NR |
| 14Q | CEO | 12Q | <0.5 | NR | NR |
| 14R | Comp | 12R | <0.5 | NR | NR |
| 14S | CEO | 12S | <0.5 | NR | NR |
| 14T | CEO | 12T | <0.5 | NR | NR |
| 14U | CEO | 12U | <0.5 | NR | NR |
| 15A | Comp | 13A | <0.5 | NR | NR |
| 15B | CEO | 13B | <0.5 | NR | NR |
| 15C | Comp | 13C | <0.5 | NR | NR |
| 15D | CEO | 13D | <0.5 | NR | NR |
| 15E | Comp | 13E | <0.5 | NR | NR |
| 15F | CEO | 13F | <0.5 | NR | NR |
| 15G | Comp | 13G | <0.5 | NR | NR |
| 15H | CEO | 13H | <0.5 | NR | NR |
| 15I | Comp | 13I | <0.5 | NR | NR |
| 15J | CEO | 13J | <0.5 | NR | NR |
| 15K | Comp | 13K | <0.5 | NR | NR |
| 15L | CEO | 13L | <0.5 | NR | NR |
| 15M | CEO | 13M | <0.5 | NR | NR |
| 15N | Comp | 13N | <0.5 | NR | NR |
| 15O | CEO | 13O | <0.5 | NR | NR |
| 15P | Comp | 13P | <0.5 | NR | NR |
| 15Q | CEO | 13Q | <0.5 | NR | NR |
| 15R | Comp | 13R | <0.5 | NR | NR |
| 15S | CEO | 13S | <0.5 | NR | NR |
| 15T | CEO | 13T | <0.5 | NR | NR |
| 15U | CEO | 13U | <0.5 | NR | NR |
| 16A | Comp | 14A | <0.5 | NR | NR |
| 16B | CEO | 14B | <0.5 | NR | NR |
| 16C | Comp | 14C | <0.5 | NR | NR |
| 16D | CEO | 14D | <0.5 | NR | NR |
| 16E | Comp | 14E | <0.5 | NR | NR |
| 16F | CEO | 14F | <0.5 | NR | NR |
| 16G | Comp | 14G | <0.5 | NR | NR |
| 16H | CEO | 14H | <0.5 | NR | NR |
| 16I | Comp | 14I | <0.5 | NR | NR |
| 16J | CEO | 14J | <0.5 | NR | NR |
| 16K | Comp | 14K | <0.5 | NR | NR |
| 16L | CEO | 14L | <0.5 | NR | NR |
| 16M | CEO | 14M | <0.5 | NR | NR |
| 16N | Comp | 14N | <0.5 | NR | NR |
| 16O | CEO | 14O | <0.5 | NR | NR |
| 16P | Comp | 14P | <0.5 | NR | NR |
| 16Q | CEO | 14Q | <0.5 | NR | NR |
| 16R | Comp | 14R | <0.5 | NR | NR |
| 16S | CEO | 14S | <0.5 | NR | NR |
| 16T | CEO | 14T | <0.5 | NR | NR |
| 16U | CEO | 14U | <0.5 | NR | NR |
| 17A | Comp | 15A | <0.5 | NR | NR |
| 17B | CEO | 15B | <0.5 | NR | NR |
| 17C | Comp | 15C | <0.5 | NR | NR |
| 17D | CEO | 15D | <0.5 | NR | NR |
| 17E | Comp | 15E | <0.5 | NR | NR |
| 17F | CEO | 15F | <0.5 | NR | NR |
| 17G | Comp | 15G | <0.5 | NR | NR |
| 17H | CEO | 15H | <0.5 | NR | NR |
| 17I | Comp | 15I | <0.5 | NR | NR |
| 17J | CEO | 15J | <0.5 | NR | NR |
| 17K | Comp | 15K | <0.5 | NR | NR |
| 17L | CEO | 15L | <0.5 | NR | NR |
| 17M | CEO | 15M | <0.5 | NR | NR |
| 17N | Comp | 15N | <0.5 | NR | NR |
| 17O | CEO | 15O | <0.5 | NR | NR |
| 17P | Comp | 15P | <0.5 | NR | NR |
| 17Q | CEO | 15Q | <0.5 | NR | NR |
| 17R | Comp | 15R | <0.5 | NR | NR |
| 17S | CEO | 15S | <0.5 | NR | NR |
| 17T | CEO | 15T | <0.5 | NR | NR |
| 17U | CEO | 15U | <0.5 | NR | NR |
| 18A | Comp | 16A | <0.5 | NR | NR |
| 18B | CEO | 16B | <0.5 | NR | NR |
| 18C | Comp | 16B | <0.5 | NR | NR |
| 18D | CEO | 16D | <0.5 | NR | NR |
| 18E | Comp | 16E | <0.5 | NR | NR |
| 18F | CEO | 16F | <0.5 | NR | NR |
| 18G | Comp | 16G | <0.5 | NR | NR |
| 18H | CEO | 16H | <0.5 | NR | NR |
| 18I | Comp | 16I | <0.5 | NR | NR |
| 18J | CEO | 16J | <0.5 | NR | NR |
| 18K | Comp | 16K | <0.5 | NR | NR |
| 18L | CEO | 16L | <0.5 | NR | NR |
| 18M | CEO | 16M | <0.5 | NR | NR |
| 18N | Comp | 16N | <0.5 | NR | NR |
| 18O | CEO | 16O | <0.5 | NR | NR |
| 18P | Comp | 16P | <0.5 | NR | NR |
| 18Q | CEO | 16Q | <0.5 | NR | NR |
| 18R | Comp | 16R | <0.5 | NR | NR |
| 18S | CEO | 16S | <0.5 | NR | NR |
| 18T | CEO | 16T | <0.5 | NR | NR |
| 18U | CEO | 16U | <0.5 | NR | NR |
| 19A | Comp | 17A | <0.5 | NR | NR |
| 19B | CEO | 17B | <0.5 | NR | NR |
| 19C | Comp | 17C | <0.5 | NR | NR |
| 19D | CEO | 17D | <0.5 | NR | NR |
| 19E | Comp | 17E | <0.5 | NR | NR |
| 19F | CEO | 17F | <0.5 | NR | NR |
| 19G | Comp | 17G | <0.5 | NR | NR |
| 19H | CEO | 17H | <0.5 | NR | NR |
| 19I | Comp | 17I | <0.5 | NR | NR |
| 19J | CEO | 17J | <0.5 | NR | NR |
| 19K | Comp | 17K | <0.5 | NR | NR |
| 19L | CEO | 17L | <0.5 | NR | NR |
| 19M | CEO | 17M | <0.5 | NR | NR |
| 19N | Comp | 17N | <0.5 | NR | NR |
| 19O | CEO | 17O | <0.5 | NR | NR |
| 19P | Comp | 17P | <0.5 | NR | NR |
| 19Q | CEO | 17Q | <0.5 | NR | NR |
| 19R | Comp | 17R | <0.5 | NR | NR |
| 19S | CEO | 17S | <0.5 | NR | NR |
| 19T | CEO | 17T | <0.5 | NR | NR |
| 19U | CEO | 17U | <0.5 | NR | NR |
| 20A | Comp | 18A | <0.5 | NR | NR |
| 20B | CEO | 18B | <0.5 | NR | NR |
| 20C | Comp | 18C | <0.5 | NR | NR |
| 20D | CEO | 18D | <0.5 | NR | NR |
| 20E | Comp | 18E | <0.5 | NR | NR |
| 20F | CEO | 18F | <0.5 | NR | NR |
| 20G | Comp | 18G | <0.5 | NR | NR |
| 20H | CEO | 18H | <0.5 | NR | NR |
| 20I | Comp | 18I | <0.5 | NR | NR |
| 20J | CEO | 18J | <0.5 | NR | NR |
| 20K | Comp | 18K | <0.5 | NR | NR |
| 20L | CEO | 18L | <0.5 | NR | NR |
| 20M | CEO | 18M | <0.5 | NR | NR |
| 20N | Comp | 18N | <0.5 | NR | NR |
| 20O | CEO | 18O | <0.5 | NR | NR |
| 20P | Comp | 18P | <0.5 | NR | NR |
| 20Q | CEO | 18Q | <0.5 | NR | NR |
| 20R | Comp | 18R | <0.5 | NR | NR |
| 20S | CEO | 18S | <0.5 | NR | NR |
| 20T | CEO | 18T | <0.5 | NR | NR |
| 20U | CEO | 18U | <0.5 | NR | NR |
| 21A | Comp | 19A | <0.5 | NR | NR |
| 21B | CEO | 19B | <0.5 | NR | NR |
| 21C | Comp | 19C | <0.5 | NR | NR |
| 21D | CEO | 19D | <0.5 | NR | NR |
| 21E | Comp | 19E | <0.5 | NR | NR |
| 21F | CEO | 19F | <0.5 | NR | NR |
| 21G | Comp | 19G | <0.5 | NR | NR |
| 21H | CEO | 19H | <0.5 | NR | NR |
| 21I | Comp | 19I | <0.5 | NR | NR |
| 21J | CEO | 19J | <0.5 | NR | NR |
| 21K | Comp | 19K | <0.5 | NR | NR |
| 21L | CEO | 19L | <0.5 | NR | NR |
| 21M | CEO | 19M | <0.5 | NR | NR |
| 21N | Comp | 19N | <0.5 | NR | NR |
| 21O | CEO | 19O | <0.5 | NR | NR |
| 21P | Comp | 19P | <0.5 | NR | NR |
| 21Q | CEO | 19Q | <0.5 | NR | NR |
| 21R | Comp | 19R | <0.5 | NR | NR |
| 21S | CEO | 19S | <0.5 | NR | NR |
| 21T | CEO | 19T | <0.5 | NR | NR |
| 21U | CEO | 20U | <0.5 | NR | NR |
| 22A | Comp | 20A | <0.5 | NR | NR |
| 22B | CEO | 20B | <0.5 | NR | NR |
| 22C | Comp | 20C | <0.5 | NR | NR |
| 22D | CEO | 20D | <0.5 | NR | NR |
| 22E | Comp | 20E | <0.5 | NR | NR |
| 22F | CEO | 20F | <0.5 | NR | NR |
| 22G | Comp | 20G | <0.5 | NR | NR |
| 22H | CEO | 20H | <0.5 | NR | NR |
| 22I | Comp | 20I | <0.5 | NR | NR |
| 22J | CEO | 20J | <0.5 | NR | NR |
| 22K | Comp | 20K | <0.5 | NR | NR |
| 22L | CEO | 20L | <0.5 | NR | NR |
| 22M | CEO | 20M | <0.5 | NR | NR |
| 22N | Comp | 20N | <0.5 | NR | NR |
| 22O | CEO | 20O | <0.5 | NR | NR |
| 22P | Comp | 20P | <0.5 | NR | NR |
| 22Q | CEO | 20Q | <0.5 | NR | NR |
| 22R | Comp | 20R | <0.5 | NR | NR |
| 22S | CEO | 20S | <0.5 | NR | NR |
| 22T | CEO | 20T | <0.5 | NR | NR |
| 22U | CEO | 21U | <0.5 | NR | NR |
| 23A | Comp | 21A | <0.5 | NR | NR |
| 23B | CEO | 21B | <0.5 | NR | NR |
| 23C | Comp | 21C | <0.5 | NR | NR |
| 23D | CEO | 21D | <0.5 | NR | NR |
| 23E | Comp | 21E | <0.5 | NR | NR |
| 23F | CEO | 21F | <0.5 | NR | NR |
| 23G | Comp | 21G | <0.5 | NR | NR |
| 23H | CEO | 21H | <0.5 | NR | NR |
| 23I | Comp | 21I | <0.5 | NR | NR |
| 23J | CEO | 21J | <0.5 | NR | NR |
| 23K | Comp | 21K | <0.5 | NR | NR |
| 23L | CEO | 21L | <0.5 | NR | NR |
| 23M | CEO | 21M | <0.5 | NR | NR |
| 23N | Comp | 21N | <0.5 | NR | NR |
| 23O | CEO | 21O | <0.5 | NR | NR |
| 23P | Comp | 21P | <0.5 | NR | NR |
| 23Q | CEO | 21Q | <0.5 | NR | NR |
| 23R | Comp | 21R | <0.5 | NR | NR |
| 23S | CEO | 21S | <0.5 | NR | NR |
| 23T | CEO | 21T | <0.5 | NR | NR |
| 23U | CEO | 21U | <0.5 | NR | NR |
| 24A | Comp | 22A | <0.5 | NR | NR |
| 24B | CEO | 22B | <0.5 | NR | NR |
| 24C | Comp | 22C | <0.5 | NR | NR |
| 24D | CEO | 22D | <0.5 | NR | NR |
| 24E | Comp | 22E | <0.5 | NR | NR |
| 24F | CEO | 22F | <0.5 | NR | NR |
| 24G | Comp | 22G | <0.5 | NR | NR |
| 24H | CEO | 22H | <0.5 | NR | NR |
| 24I | Comp | 22I | <0.5 | NR | NR |
| 24J | CEO | 22J | <0.5 | NR | NR |
| 24K | Comp | 22K | <0.5 | NR | NR |
| 24L | CEO | 22L | <0.5 | NR | NR |
| 24M | CEO | 22M | <0.5 | NR | NR |
| 24N | Comp | 22N | <0.5 | NR | NR |
| 24O | CEO | 22O | <0.5 | NR | NR |
| 24P | Comp | 22P | <0.5 | NR | NR |
| 24Q | CEO | 22Q | <0.5 | NR | NR |
| 24R | Comp | 22R | <0.5 | NR | NR |
| 24S | CEO | 22S | <0.5 | NR | NR |
| 24T | CEO | 22T | <0.5 | NR | NR |
| 24U | CEO | 22U | <0.5 | NR | NR |
| 25A | Comp | 23A | <0.5 | NR | NR |
| 25B | CEO | 23B | <0.5 | NR | NR |
| 25C | Comp | 23C | <0.5 | NR | NR |
| 25D | CEO | 23D | <0.5 | NR | NR |
| 25E | Comp | 23E | <0.5 | NR | NR |
| 25F | CEO | 23F | <0.5 | NR | NR |
| 25G | Comp | 23G | <0.5 | NR | NR |
| 25H | CEO | 23H | <0.5 | NR | NR |
| 25I | Comp | 23I | <0.5 | NR | NR |
| 25J | CEO | 23J | <0.5 | NR | NR |
| 25K | Comp | 23K | <0.5 | NR | NR |
| 25L | CEO | 23L | <0.5 | NR | NR |
| 25M | CEO | 23M | <0.5 | NR | NR |
| 25N | Comp | 23N | <0.5 | NR | NR |
| 25O | CEO | 23O | <0.5 | NR | NR |
| 25P | Comp | 23P | <0.5 | NR | NR |
| 25Q | CEO | 23Q | <0.5 | NR | NR |
| 25R | Comp | 23R | <0.5 | NR | NR |
| 25S | CEO | 23S | <0.5 | NR | NR |
| 25T | CEO | 23T | <0.5 | NR | NR |
| 26U | CEO | 23U | <0.5 | NR | NR |
| 26A | Comp | 24A | <0.5 | NR | NR |
| 26B | CEO | 24B | <0.5 | NR | NR |
| 26C | Comp | 24C | <0.5 | NR | NR |
| 26D | CEO | 24D | <0.5 | NR | NR |
| 26E | Comp | 24E | <0.5 | NR | NR |
| 26F | CEO | 24F | <0.5 | NR | NR |
| 26G | Comp | 24G | <0.5 | NR | NR |
| 26H | CEO | 24H | <0.5 | NR | NR |
| 26I | Comp | 24I | <0.5 | NR | NR |
| 26J | CEO | 24J | <0.5 | NR | NR |
| 26K | Comp | 24K | <0.5 | NR | NR |
| 26L | CEO | 24L | <0.5 | NR | NR |
| 26M | CEO | 24M | <0.5 | NR | NR |
| 26N | Comp | 24N | <0.5 | NR | NR |
| 26O | CEO | 24O | <0.5 | NR | NR |
| 26P | Comp | 24P | <0.5 | NR | NR |
| 26Q | CEO | 24Q | <0.5 | NR | NR |
| 26R | Comp | 24R | <0.5 | NR | NR |
| 26S | CEO | 24S | <0.5 | NR | NR |
| 26T | CEO | 24T | <0.5 | NR | NR |
| 26U | CEO | 24U | <0.5 | NR | NR |
| 27A | Comp | 25A | <0.5 | NR | NR |
| 27B | CEO | 25B | <0.5 | NR | NR |
| 27C | Comp | 25C | <0.5 | NR | NR |
| 27D | CEO | 25D | <0.5 | NR | NR |
| 27E | Comp | 25E | <0.5 | NR | NR |
| 27F | CEO | 25F | <0.5 | NR | NR |
| 27G | Comp | 25G | <0.5 | NR | NR |
| 27H | CEO | 25H | <0.5 | NR | NR |
| 27I | Comp | 25I | <0.5 | NR | NR |
| 27J | CEO | 25J | <0.5 | NR | NR |
| 27K | Comp | 25K | <0.5 | NR | NR |
| 27L | CEO | 25L | <0.5 | NR | NR |
| 27M | CEO | 25M | <0.5 | NR | NR |
| 27N | Comp | 25N | <0.5 | NR | NR |
| 27O | CEO | 25O | <0.5 | NR | NR |
| 27P | Comp | 25P | <0.5 | NR | NR |
| 27Q | CEO | 25Q | <0.5 | NR | NR |
| 27R | Comp | 25R | <0.5 | NR | NR |
| 27S | CEO | 25S | <0.5 | NR | NR |
| 27T | CEO | 25T | <0.5 | NR | NR |
| 27U | CEO | 25U | <0.5 | NR | NR |
| 28A | Comp | 26A | <0.5 | NR | NR |
| 28B | CEO | 26B | <0.5 | NR | NR |
| 28C | Comp | 26C | <0.5 | NR | NR |
| 28D | CEO | 26D | <0.5 | NR | NR |
| 28E | Comp | 26E | <0.5 | NR | NR |
| 28F | CEO | 26F | <0.5 | NR | NR |
| 28G | Comp | 26G | <0.5 | NR | NR |
| 28H | CEO | 26H | <0.5 | NR | NR |
| 28I | Comp | 26I | <0.5 | NR | NR |
| 28J | CEO | 26J | <0.5 | NR | NR |
| 28K | Comp | 26K | <0.5 | NR | NR |
| 28L | CEO | 26L | <0.5 | NR | NR |
| 28M | CEO | 26M | <0.5 | NR | NR |
| 28N | Comp | 26N | <0.5 | NR | NR |
| 28O | CEO | 26O | <0.5 | NR | NR |
| 28P | Comp | 26P | <0.5 | NR | NR |
| 28Q | CEO | 26Q | <0.5 | NR | NR |
| 28R | Comp | 26R | <0.5 | NR | NR |
| 28S | CEO | 26S | <0.5 | NR | NR |
| 28T | CEO | 26T | <0.5 | NR | NR |
| 28U | CEO | 26U | <0.5 | NR | NR |
| 29A | Comp | 1A | >0.2 to <0.5 | >85% | >105% |
| 29B | CEO | 1B | >0.2 to <0.5 | >85% | >105% |
| 29C | Comp | 1C | >0.2 to <0.5 | >85% | >105% |
| 29D | CEO | 1D | >0.2 to <0.5 | >85% | >105% |
| 29E | Comp | 1E | >0.2 to <0.5 | >85% | >105% |
| 29F | CEO | 1F | >0.2 to <0.5 | >85% | >105% |
| 29G | Comp | 1G | >0.2 to <0.5 | >85% | >105% |
| 29H | CEO | 1H | >0.2 to <0.5 | >85% | >105% |
| 29I | Comp | 1I | >0.2 to <0.5 | >85% | >105% |
| 29J | CEO | 1J | >0.2 to <0.5 | >85% | >105% |
| 29K | Comp | 1K | >0.2 to <0.5 | >85% | >105% |
| 29L | CEO | 1L | >0.2 to <0.5 | >85% | >105% |
| 29M | CEO | 1M | >0.2 to <0.5 | >85% | >105% |
| 29N | Comp | 1N | >0.2 to <0.5 | >85% | >105% |
| 29O | CEO | 1O | >0.2 to <0.5 | >85% | >105% |
| 29P | Comp | 1P | >0.2 to <0.5 | >85% | >105% |
| 29Q | CEO | 1Q | >0.2 to <0.5 | >85% | >105% |
| 29R | Comp | 1R | >0.2 to <0.5 | >85% | >105% |
| 29S | CEO | 1S | >0.2 to <0.5 | >85% | >105% |
| 29T | CEO | 1T | >0.2 to <0.5 | >85% | >105% |
| 29U | CEO | 1U | >0.2 to <0.5 | >85% | >105% |
| 30A | Comp | 2A | >0.2 to <0.5 | >85% | >105% |
| 30B | CEO | 2B | >0.2 to <0.5 | >85% | >105% |
| 30C | Comp | 2C | >0.2 to <0.5 | >85% | >105% |
| 30D | CEO | 2D | >0.2 to <0.5 | >85% | >105% |
| 30E | Comp | 2E | >0.2 to <0.5 | >85% | >105% |
| 30F | CEO | 2F | >0.2 to <0.5 | >85% | >105% |
| 30G | Comp | 2G | >0.2 to <0.5 | >85% | >105% |
| 30H | CEO | 2H | >0.2 to <0.5 | >85% | >105% |
| 30I | Comp | 2I | >0.2 to <0.5 | >85% | >105% |
| 30J | CEO | 2J | >0.2 to <0.5 | >85% | >105% |
| 30K | Comp | 2K | >0.2 to <0.5 | >85% | >105% |
| 30L | CEO | 2L | >0.2 to <0.5 | >85% | >105% |
| 30M | CEO | 2M | >0.2 to <0.5 | >85% | >105% |
| 30N | Comp | 2N | >0.2 to <0.5 | >85% | >105% |
| 30O | CEO | 2O | >0.2 to <0.5 | >85% | >105% |
| 30P | Comp | 2P | >0.2 to <0.5 | >85% | >105% |
| 30Q | CEO | 2Q | >0.2 to <0.5 | >85% | >105% |
| 30R | Comp | 2R | >0.2 to <0.5 | >85% | >105% |
| 30S | CEO | 2S | >0.2 to <0.5 | >85% | >105% |
| 30T | CEO | 2T | >0.2 to <0.5 | >85% | >105% |
| 30U | CEO | 2U | >0.2 to <0.5 | >85% | >105% |
| 31A | Comp | 3A | >0.2 to <0.5 | >85% | >105% |
| 31B | CEO | 3B | >0.2 to <0.5 | >85% | >105% |
| 31C | Comp | 3C | >0.2 to <0.5 | >85% | >105% |
| 31D | CEO | 3D | >0.2 to <0.5 | >85% | >105% |
| 31E | Comp | 3E | >0.2 to <0.5 | >85% | >105% |
| 31F | CEO | 3F | >0.2 to <0.5 | >85% | >105% |
| 31G | Comp | 3G | >0.2 to <0.5 | >85% | >105% |
| 31H | CEO | 3H | >0.2 to <0.5 | >85% | >105% |
| 31I | Comp | 3I | >0.2 to <0.5 | >85% | >105% |
| 31J | CEO | 3J | >0.2 to <0.5 | >85% | >105% |
| 31K | Comp | 3K | >0.2 to <0.5 | >85% | >105% |
| 31L | CEO | 3L | >0.2 to <0.5 | >85% | >105% |
| 31M | CEO | 3M | >0.2 to <0.5 | >85% | >105% |
| 31N | Comp | 3N | >0.2 to <0.5 | >85% | >105% |
| 31O | CEO | 3O | >0.2 to <0.5 | >85% | >105% |
| 31P | Comp | 3P | >0.2 to <0.5 | >85% | >105% |
| 31Q | CEO | 3Q | >0.2 to <0.5 | >85% | >105% |
| 31R | Comp | 3R | >0.2 to <0.5 | >85% | >105% |
| 31S | CEO | 3S | >0.2 to <0.5 | >85% | >105% |
| 31T | CEO | 3T | >0.2 to <0.5 | >85% | >105% |
| 31U | CEO | 3U | >0.2 to <0.5 | >85% | >105% |
| 32A | Comp | 4A | >0.2 to <0.5 | >85% | >105% |
| 32B | CEO | 4B | >0.2 to <0.5 | >85% | >105% |
| 32C | Comp | 4C | >0.2 to <0.5 | >85% | >105% |
| 32D | CEO | 4D | >0.2 to <0.5 | >85% | >105% |
| 32E | Comp | 4E | >0.2 to <0.5 | >85% | >105% |
| 32F | CEO | 4F | >0.2 to <0.5 | >85% | >105% |
| 32G | Comp | 4G | >0.2 to <0.5 | >85% | >105% |
| 32H | CEO | 4H | >0.2 to <0.5 | >85% | >105% |
| 32I | Comp | 4I | >0.2 to <0.5 | >85% | >105% |
| 32J | CEO | 4J | >0.2 to <0.5 | >85% | >105% |
| 32K | Comp | 4K | >0.2 to <0.5 | >85% | >105% |
| 32L | CEO | 4L | >0.2 to <0.5 | >85% | >105% |
| 32M | CEO | 4M | >0.2 to <0.5 | >85% | >105% |
| 32N | Comp | 4N | >0.2 to <0.5 | >85% | >105% |
| 32O | CEO | 4O | >0.2 to <0.5 | >85% | >105% |
| 32P | Comp | 4P | >0.2 to <0.5 | >85% | >105% |
| 32Q | CEO | 4Q | >0.2 to <0.5 | >85% | >105% |
| 32R | Comp | 4R | >0.2 to <0.5 | >85% | >105% |
| 32S | CEO | 4S | >0.2 to <0.5 | >85% | >105% |
| 32T | CEO | 4T | >0.2 to <0.5 | >85% | >105% |
| 32U | CEO | 4U | >0.2 to <0.5 | >85% | >105% |
| 33A | Comp | 5A | >0.2 to <0.5 | >85% | >105% |
| 33B | CEO | 5B | >0.2 to <0.5 | >85% | >105% |
| 33C | Comp | 5C | >0.2 to <0.5 | >85% | >105% |
| 33D | CEO | 5D | >0.2 to <0.5 | >85% | >105% |
| 33E | Comp | 5E | >0.2 to <0.5 | >85% | >105% |
| 33F | CEO | 5F | >0.2 to <0.5 | >85% | >105% |
| 33G | Comp | 5G | >0.2 to <0.5 | >85% | >105% |
| 33H | CEO | 5H | >0.2 to <0.5 | >85% | >105% |
| 33I | Comp | 5I | >0.2 to <0.5 | >85% | >105% |
| 33J | CEO | 5J | >0.2 to <0.5 | >85% | >105% |
| 33K | Comp | 5K | >0.2 to <0.5 | >85% | >105% |
| 33L | CEO | 5L | >0.2 to <0.5 | >85% | >105% |
| 33M | CEO | 5M | >0.2 to <0.5 | >85% | >105% |
| 33N | Comp | 5N | >0.2 to <0.5 | >85% | >105% |
| 33O | CEO | 5O | >0.2 to <0.5 | >85% | >105% |
| 33P | Comp | 5P | >0.2 to <0.5 | >85% | >105% |
| 33Q | CEO | 5Q | >0.2 to <0.5 | >85% | >105% |
| 33R | Comp | 5R | >0.2 to <0.5 | >85% | >105% |
| 33S | CEO | 5S | >0.2 to <0.5 | >85% | >105% |
| 33T | CEO | 5T | >0.2 to <0.5 | >85% | >105% |
| 33U | CEO | 5U | >0.2 to <0.5 | >85% | >105% |
| 34A | Comp | 6A | >0.2 to <0.5 | >85% | >105% |
| 34B | CEO | 6B | >0.2 to <0.5 | >85% | >105% |
| 34C | Comp | 6C | >0.2 to <0.5 | >85% | >105% |
| 34D | CEO | 6D | >0.2 to <0.5 | >85% | >105% |
| 34E | Comp | 6E | >0.2 to <0.5 | >85% | >105% |
| 34F | CEO | 6F | >0.2 to <0.5 | >85% | >105% |
| 34G | Comp | 6G | >0.2 to <0.5 | >85% | >105% |
| 34H | CEO | 6H | >0.2 to <0.5 | >85% | >105% |
| 34I | Comp | 6I | >0.2 to <0.5 | >85% | >105% |
| 34J | CEO | 6J | >0.2 to <0.5 | >85% | >105% |
| 34K | Comp | 6K | >0.2 to <0.5 | >85% | >105% |
| 34L | CEO | 6L | >0.2 to <0.5 | >85% | >105% |
| 34M | CEO | 6M | >0.2 to <0.5 | >85% | >105% |
| 34N | Comp | 6N | >0.2 to <0.5 | >85% | >105% |
| 34O | CEO | 6O | >0.2 to <0.5 | >85% | >105% |
| 34P | Comp | 6P | >0.2 to <0.5 | >85% | >105% |
| 34Q | CEO | 6Q | >0.2 to <0.5 | >85% | >105% |
| 34R | Comp | 6R | >0.2 to <0.5 | >85% | >105% |
| 34S | CEO | 6S | >0.2 to <0.5 | >85% | >105% |
| 34T | CEO | 6T | >0.2 to <0.5 | >85% | >105% |
| 34U | CEO | 6U | >0.2 to <0.5 | >85% | >105% |
| 35A | Comp | 7A | >0.2 to <0.5 | >85% | >105% |
| 35B | CEO | 7B | >0.2 to <0.5 | >85% | >105% |
| 35C | Comp | 7C | >0.2 to <0.5 | >85% | >105% |
| 35D | CEO | 7D | >0.2 to <0.5 | >85% | >105% |
| 35E | Comp | 7E | >0.2 to <0.5 | >85% | >105% |
| 35F | CEO | 7F | >0.2 to <0.5 | >85% | >105% |
| 35G | Comp | 7G | >0.2 to <0.5 | >85% | >105% |
| 35H | CEO | 7H | >0.2 to <0.5 | >85% | >105% |
| 35I | Comp | 7I | >0.2 to <0.5 | >85% | >105% |
| 35J | CEO | 7J | >0.2 to <0.5 | >85% | >105% |
| 35K | Comp | 7K | >0.2 to <0.5 | >85% | >105% |
| 35L | CEO | 7L | >0.2 to <0.5 | >85% | >105% |
| 35M | CEO | 7M | >0.2 to <0.5 | >85% | >105% |
| 35N | Comp | 7N | >0.2 to <0.5 | >85% | >105% |
| 35O | CEO | 7O | >0.2 to <0.5 | >85% | >105% |
| 35P | Comp | 7P | >0.2 to <0.5 | >85% | >105% |
| 35Q | CEO | 7Q | >0.2 to <0.5 | >85% | >105% |
| 35R | Comp | 7R | >0.2 to <0.5 | >85% | >105% |
| 35S | CEO | 7S | >0.2 to <0.5 | >85% | >105% |
| 35T | CEO | 7T | >0.2 to <0.5 | >85% | >105% |
| 35U | CEO | 7U | >0.2 to <0.5 | >85% | >105% |
| 36A | Comp | 8A | >0.2 to <0.5 | >85% | >105% |
| 36B | CEO | 8B | >0.2 to <0.5 | >85% | >105% |
| 36C | Comp | 8C | >0.2 to <0.5 | >85% | >105% |
| 36D | CEO | 8D | >0.2 to <0.5 | >85% | >105% |
| 36E | Comp | 8E | >0.2 to <0.5 | >85% | >105% |
| 36F | CEO | 8F | >0.2 to <0.5 | >85% | >105% |
| 36G | Comp | 8G | >0.2 to <0.5 | >85% | >105% |
| 36H | CEO | 8H | >0.2 to <0.5 | >85% | >105% |
| 36I | Comp | 8I | >0.2 to <0.5 | >85% | >105% |
| 36J | CEO | 8J | >0.2 to <0.5 | >85% | >105% |
| 36K | Comp | 8K | >0.2 to <0.5 | >85% | >105% |
| 36L | CEO | 8L | >0.2 to <0.5 | >85% | >105% |
| 36M | CEO | 8M | >0.2 to <0.5 | >85% | >105% |
| 36N | Comp | 8N | >0.2 to <0.5 | >85% | >105% |
| 36O | CEO | 8O | >0.2 to <0.5 | >85% | >105% |
| 36P | Comp | 8P | >0.2 to <0.5 | >85% | >105% |
| 36Q | CEO | 8Q | >0.2 to <0.5 | >85% | >105% |
| 36R | Comp | 8R | >0.2 to <0.5 | >85% | >105% |
| 36S | CEO | 8S | >0.2 to <0.5 | >85% | >105% |
| 36T | CEO | 8T | >0.2 to <0.5 | >85% | >105% |
| 36U | CEO | 8U | >0.2 to <0.5 | >85% | >105% |
| 37A | Comp | 9A | >0.2 to <0.5 | >85% | >105% |
| 37B | CEO | 9B | >0.2 to <0.5 | >85% | >105% |
| 37C | Comp | 9C | >0.2 to <0.5 | >85% | >105% |
| 37D | CEO | 9D | >0.2 to <0.5 | >85% | >105% |
| 37E | Comp | 9E | >0.2 to <0.5 | >85% | >105% |
| 37F | CEO | 9F | >0.2 to <0.5 | >85% | >105% |
| 37G | Comp | 9G | >0.2 to <0.5 | >85% | >105% |
| 37H | CEO | 9H | >0.2 to <0.5 | >85% | >105% |
| 37I | Comp | 9I | >0.2 to <0.5 | >85% | >105% |
| 37J | CEO | 9J | >0.2 to <0.5 | >85% | >105% |
| 37K | Comp | 9K | >0.2 to <0.5 | >85% | >105% |
| 37L | CEO | 9L | >0.2 to <0.5 | >85% | >105% |
| 37M | CEO | 9M | >0.2 to <0.5 | >85% | >105% |
| 37N | Comp | 9N | >0.2 to <0.5 | >85% | >105% |
| 37O | CEO | 9O | >0.2 to <0.5 | >85% | >105% |
| 37P | Comp | 9P | >0.2 to <0.5 | >85% | >105% |
| 37Q | CEO | 9Q | >0.2 to <0.5 | >85% | >105% |
| 37R | Comp | 9R | >0.2 to <0.5 | >85% | >105% |
| 37S | CEO | 9S | >0.2 to <0.5 | >85% | >105% |
| 37T | CEO | 9T | >0.2 to <0.5 | >85% | >105% |
| 37U | CEO | 9U | >0.2 to <0.5 | >85% | >105% |
| 38A | Comp | 10A | >0.2 to <0.5 | >85% | >105% |
| 38B | CEO | 10B | >0.2 to <0.5 | >85% | >105% |
| 38C | Comp | 10C | >0.2 to <0.5 | >85% | >105% |
| 38D | CEO | 10D | >0.2 to <0.5 | >85% | >105% |
| 38E | Comp | 10E | >0.2 to <0.5 | >85% | >105% |
| 38F | CEO | 10F | >0.2 to <0.5 | >85% | >105% |
| 38G | Comp | 10G | >0.2 to <0.5 | >85% | >105% |
| 38H | CEO | 10H | >0.2 to <0.5 | >85% | >105% |
| 38I | Comp | 10I | >0.2 to <0.5 | >85% | >105% |
| 38J | CEO | 10J | >0.2 to <0.5 | >85% | >105% |
| 38K | Comp | 10K | >0.2 to <0.5 | >85% | >105% |
| 38L | CEO | 10L | >0.2 to <0.5 | >85% | >105% |
| 38M | CEO | 10M | >0.2 to <0.5 | >85% | >105% |
| 38N | Comp | 10N | >0.2 to <0.5 | >85% | >105% |
| 38O | CEO | 10O | >0.2 to <0.5 | >85% | >105% |
| 38P | Comp | 10P | >0.2 to <0.5 | >85% | >105% |
| 38Q | CEO | 10Q | >0.2 to <0.5 | >85% | >105% |
| 38R | Comp | 10R | >0.2 to <0.5 | >85% | >105% |
| 38S | CEO | 10S | >0.2 to <0.5 | >85% | >105% |
| 38T | CEO | 10T | >0.2 to <0.5 | >85% | >105% |
| 38U | CEO | 10U | >0.2 to <0.5 | >85% | >105% |
| 39A | Comp | 11A | >0.2 to <0.5 | >85% | >105% |
| 39B | CEO | 11B | >0.2 to <0.5 | >85% | >105% |
| 39C | Comp | 11C | >0.2 to <0.5 | >85% | >105% |
| 39D | CEO | 11D | >0.2 to <0.5 | >85% | >105% |
| 39E | Comp | 11E | >0.2 to <0.5 | >85% | >105% |
| 39F | CEO | 11F | >0.2 to <0.5 | >85% | >105% |
| 39G | Comp | 11G | >0.2 to <0.5 | >85% | >105% |
| 39H | CEO | 11H | >0.2 to <0.5 | >85% | >105% |
| 39I | Comp | 11I | >0.2 to <0.5 | >85% | >105% |
| 39J | CEO | 11J | >0.2 to <0.5 | >85% | >105% |
| 39K | Comp | 11K | >0.2 to <0.5 | >85% | >105% |
| 39L | CEO | 11L | >0.2 to <0.5 | >85% | >105% |
| 39M | CEO | 11M | >0.2 to <0.5 | >85% | >105% |
| 39N | Comp | 11N | >0.2 to <0.5 | >85% | >105% |
| 39O | CEO | 11O | >0.2 to <0.5 | >85% | >105% |
| 39P | Comp | 11P | >0.2 to <0.5 | >85% | >105% |
| 39Q | CEO | 11Q | >0.2 to <0.5 | >85% | >105% |
| 39R | Comp | 11R | >0.2 to <0.5 | >85% | >105% |
| 39S | CEO | 11S | >0.2 to <0.5 | >85% | >105% |
| 39T | CEO | 11T | >0.2 to <0.5 | >85% | >105% |
| 39U | CEO | 11U | >0.2 to <0.5 | >85% | >105% |
| 40A | Comp | 12A | >0.2 to <0.5 | >85% | >105% |
| 40B | CEO | 12B | >0.2 to <0.5 | >85% | >105% |
| 40C | Comp | 12C | >0.2 to <0.5 | >85% | >105% |
| 40D | CEO | 12D | >0.2 to <0.5 | >85% | >105% |
| 40E | Comp | 12E | >0.2 to <0.5 | >85% | >105% |
| 40F | CEO | 12F | >0.2 to <0.5 | >85% | >105% |
| 40G | Comp | 12G | >0.2 to <0.5 | >85% | >105% |
| 40H | CEO | 12H | >0.2 to <0.5 | >85% | >105% |
| 40I | Comp | 12I | >0.2 to <0.5 | >85% | >105% |
| 40J | CEO | 12J | >0.2 to <0.5 | >85% | >105% |
| 40K | Comp | 12K | >0.2 to <0.5 | >85% | >105% |
| 40L | CEO | 12L | >0.2 to <0.5 | >85% | >105% |
| 40M | CEO | 12M | >0.2 to <0.5 | >85% | >105% |
| 40N | Comp | 12N | >0.2 to <0.5 | >85% | >105% |
| 40O | CEO | 12O | >0.2 to <0.5 | >85% | >105% |
| 40P | Comp | 12P | >0.2 to <0.5 | >85% | >105% |
| 40Q | CEO | 12Q | >0.2 to <0.5 | >85% | >105% |
| 40R | Comp | 12R | >0.2 to <0.5 | >85% | >105% |
| 40S | CEO | 12S | >0.2 to <0.5 | >85% | >105% |
| 40T | CEO | 12T | >0.2 to <0.5 | >85% | >105% |
| 40U | CEO | 12U | >0.2 to <0.5 | >85% | >105% |
| 41A | Comp | 13A | >0.2 to <0.5 | >85% | >105% |
| 41B | CEO | 13B | >0.2 to <0.5 | >85% | >105% |
| 41C | Comp | 13C | >0.2 to <0.5 | >85% | >105% |
| 41D | CEO | 13D | >0.2 to <0.5 | >85% | >105% |
| 41E | Comp | 13E | >0.2 to <0.5 | >85% | >105% |
| 41F | CEO | 13F | >0.2 to <0.5 | >85% | >105% |
| 41G | Comp | 13G | >0.2 to <0.5 | >85% | >105% |
| 41H | CEO | 13H | >0.2 to <0.5 | >85% | >105% |
| 41I | Comp | 13I | >0.2 to <0.5 | >85% | >105% |
| 41J | CEO | 13J | >0.2 to <0.5 | >85% | >105% |
| 41K | Comp | 13K | >0.2 to <0.5 | >85% | >105% |
| 41L | CEO | 13L | >0.2 to <0.5 | >85% | >105% |
| 41M | CEO | 13M | >0.2 to <0.5 | >85% | >105% |
| 41N | Comp | 13N | >0.2 to <0.5 | >85% | >105% |
| 41O | CEO | 13O | >0.2 to <0.5 | >85% | >105% |
| 41P | Comp | 13P | >0.2 to <0.5 | >85% | >105% |
| 41Q | CEO | 13Q | >0.2 to <0.5 | >85% | >105% |
| 41R | Comp | 13R | >0.2 to <0.5 | >85% | >105% |
| 41S | CEO | 13S | >0.2 to <0.5 | >85% | >105% |
| 41T | CEO | 13T | >0.2 to <0.5 | >85% | >105% |
| 41U | CEO | 13U | >0.2 to <0.5 | >85% | >105% |
| 42A | Comp | 14A | >0.2 to <0.5 | >85% | >105% |
| 42B | CEO | 14B | >0.2 to <0.5 | >85% | >105% |
| 42C | Comp | 14C | >0.2 to <0.5 | >85% | >105% |
| 42D | CEO | 14D | >0.2 to <0.5 | >85% | >105% |
| 42E | Comp | 14E | >0.2 to <0.5 | >85% | >105% |
| 42F | CEO | 14F | >0.2 to <0.5 | >85% | >105% |
| 42G | Comp | 14G | >0.2 to <0.5 | >85% | >105% |
| 42H | CEO | 14H | >0.2 to <0.5 | >85% | >105% |
| 42I | Comp | 14I | >0.2 to <0.5 | >85% | >105% |
| 42J | CEO | 14J | >0.2 to <0.5 | >85% | >105% |
| 42K | Comp | 14K | >0.2 to <0.5 | >85% | >105% |
| 42L | CEO | 14L | >0.2 to <0.5 | >85% | >105% |
| 42M | CEO | 14M | >0.2 to <0.5 | >85% | >105% |
| 42N | Comp | 14N | >0.2 to <0.5 | >85% | >105% |
| 42O | CEO | 14O | >0.2 to <0.5 | >85% | >105% |
| 42P | Comp | 14P | >0.2 to <0.5 | >85% | >105% |
| 42Q | CEO | 14Q | >0.2 to <0.5 | >85% | >105% |
| 42R | Comp | 14R | >0.2 to <0.5 | >85% | >105% |
| 42S | CEO | 14S | >0.2 to <0.5 | >85% | >105% |
| 42T | CEO | 14T | >0.2 to <0.5 | >85% | >105% |
| 42U | CEO | 14U | >0.2 to <0.5 | >85% | >105% |
| 43A | Comp | 15A | >0.2 to <0.5 | >85% | >105% |
| 43B | CEO | 15B | >0.2 to <0.5 | >85% | >105% |
| 43C | Comp | 15C | >0.2 to <0.5 | >85% | >105% |
| 43D | CEO | 15D | >0.2 to <0.5 | >85% | >105% |
| 43E | Comp | 15E | >0.2 to <0.5 | >85% | >105% |
| 43F | CEO | 15F | >0.2 to <0.5 | >85% | >105% |
| 43G | Comp | 15G | >0.2 to <0.5 | >85% | >105% |
| 43H | CEO | 15H | >0.2 to <0.5 | >85% | >105% |
| 43I | Comp | 15I | >0.2 to <0.5 | >85% | >105% |
| 43J | CEO | 15J | >0.2 to <0.5 | >85% | >105% |
| 43K | Comp | 15K | >0.2 to <0.5 | >85% | >105% |
| 43L | CEO | 15L | >0.2 to <0.5 | >85% | >105% |
| 43M | CEO | 15M | >0.2 to <0.5 | >85% | >105% |
| 43N | Comp | 15N | >0.2 to <0.5 | >85% | >105% |
| 43O | CEO | 15O | >0.2 to <0.5 | >85% | >105% |
| 43P | Comp | 15P | >0.2 to <0.5 | >85% | >105% |
| 43Q | CEO | 15Q | >0.2 to <0.5 | >85% | >105% |
| 43R | Comp | 15R | >0.2 to <0.5 | >85% | >105% |
| 43S | CEO | 15S | >0.2 to <0.5 | >85% | >105% |
| 43T | CEO | 15T | >0.2 to <0.5 | >85% | >105% |
| 43U | CEO | 15U | >0.2 to <0.5 | >85% | >105% |
| 44A | Comp | 16A | >0.2 to <0.5 | >85% | >105% |
| 44B | CEO | 16B | >0.2 to <0.5 | >85% | >105% |
| 44C | Comp | 16C | >0.2 to <0.5 | >85% | >105% |
| 44D | CEO | 16D | >0.2 to <0.5 | >85% | >105% |
| 44E | Comp | 16E | >0.2 to <0.5 | >85% | >105% |
| 44F | CEO | 16F | >0.2 to <0.5 | >85% | >105% |
| 44G | Comp | 16G | >0.2 to <0.5 | >85% | >105% |
| 44H | CEO | 16H | >0.2 to <0.5 | >85% | >105% |
| 44I | Comp | 16I | >0.2 to <0.5 | >85% | >105% |
| 44J | CEO | 16J | >0.2 to <0.5 | >85% | >105% |
| 44K | Comp | 16K | >0.2 to <0.5 | >85% | >105% |
| 44L | CEO | 16L | >0.2 to <0.5 | >85% | >105% |
| 44M | CEO | 16M | >0.2 to <0.5 | >85% | >105% |
| 44N | Comp | 16N | >0.2 to <0.5 | >85% | >105% |
| 44O | CEO | 16O | >0.2 to <0.5 | >85% | >105% |
| 44P | Comp | 16P | >0.2 to <0.5 | >85% | >105% |
| 44Q | CEO | 16Q | >0.2 to <0.5 | >85% | >105% |
| 44R | Comp | 16R | >0.2 to <0.5 | >85% | >105% |
| 44S | CEO | 16S | >0.2 to <0.5 | >85% | >105% |
| 44T | CEO | 16T | >0.2 to <0.5 | >85% | >105% |
| 44U | CEO | 16U | >0.2 to <0.5 | >85% | >105% |
| 19A | Comp | 17A | >0.2 to <0.5 | >85% | >105% |
| 45B | CEO | 17B | >0.2 to <0.5 | >85% | >105% |
| 45C | Comp | 17C | >0.2 to <0.5 | >85% | >105% |
| 45D | CEO | 17D | >0.2 to <0.5 | >85% | >105% |
| 45E | Comp | 17E | >0.2 to <0.5 | >85% | >105% |
| 45F | CEO | 17F | >0.2 to <0.5 | >85% | >105% |
| 45G | Comp | 17G | >0.2 to <0.5 | >85% | >105% |
| 45H | CEO | 17H | >0.2 to <0.5 | >85% | >105% |
| 45I | Comp | 17I | >0.2 to <0.5 | >85% | >105% |
| 45J | CEO | 17J | >0.2 to <0.5 | >85% | >105% |
| 45K | Comp | 17K | >0.2 to <0.5 | >85% | >105% |
| 45L | CEO | 17L | >0.2 to <0.5 | >85% | >105% |
| 45M | CEO | 17M | >0.2 to <0.5 | >85% | >105% |
| 45N | Comp | 17N | >0.2 to <0.5 | >85% | >105% |
| 45O | CEO | 17O | >0.2 to <0.5 | >85% | >105% |
| 45P | Comp | 17P | >0.2 to <0.5 | >85% | >105% |
| 45Q | CEO | 17Q | >0.2 to <0.5 | >85% | >105% |
| 45R | Comp | 17R | >0.2 to <0.5 | >85% | >105% |
| 45S | CEO | 17S | >0.2 to <0.5 | >85% | >105% |
| 45T | CEO | 17T | >0.2 to <0.5 | >85% | >105% |
| 45U | CEO | 17U | >0.2 to <0.5 | >85% | >105% |
| 46A | Comp | 18A | >0.2 to <0.5 | >85% | >105% |
| 46B | CEO | 18B | >0.2 to <0.5 | >85% | >105% |
| 46C | Comp | 18C | >0.2 to <0.5 | >85% | >105% |
| 46D | CEO | 18D | >0.2 to <0.5 | >85% | >105% |
| 46E | Comp | 18E | >0.2 to <0.5 | >85% | >105% |
| 46F | CEO | 18F | >0.2 to <0.5 | >85% | >105% |
| 46G | Comp | 18G | >0.2 to <0.5 | >85% | >105% |
| 46H | CEO | 18H | >0.2 to <0.5 | >85% | >105% |
| 46I | Comp | 18I | >0.2 to <0.5 | >85% | >105% |
| 46J | CEO | 18J | >0.2 to <0.5 | >85% | >105% |
| 46K | Comp | 18K | >0.2 to <0.5 | >85% | >105% |
| 46L | CEO | 18L | >0.2 to <0.5 | >85% | >105% |
| 46M | CEO | 18M | >0.2 to <0.5 | >85% | >105% |
| 46N | Comp | 18N | >0.2 to <0.5 | >85% | >105% |
| 46O | CEO | 18O | >0.2 to <0.5 | >85% | >105% |
| 46P | Comp | 18P | >0.2 to <0.5 | >85% | >105% |
| 46Q | CEO | 18Q | >0.2 to <0.5 | >85% | >105% |
| 46R | Comp | 18R | >0.2 to <0.5 | >85% | >105% |
| 46S | CEO | 18S | >0.2 to <0.5 | >85% | >105% |
| 46T | CEO | 18T | >0.2 to <0.5 | >85% | >105% |
| 46U | CEO | 18U | >0.2 to <0.5 | >85% | >105% |
| 47A | Comp | 19A | >0.2 to <0.5 | >85% | >105% |
| 47B | CEO | 19B | >0.2 to <0.5 | >85% | >105% |
| 47C | Comp | 19C | >0.2 to <0.5 | >85% | >105% |
| 47D | CEO | 19D | >0.2 to <0.5 | >85% | >105% |
| 47E | Comp | 19E | >0.2 to <0.5 | >85% | >105% |
| 47F | CEO | 19F | >0.2 to <0.5 | >85% | >105% |
| 47G | Comp | 19G | >0.2 to <0.5 | >85% | >105% |
| 47H | CEO | 19H | >0.2 to <0.5 | >85% | >105% |
| 47I | Comp | 19I | >0.2 to <0.5 | >85% | >105% |
| 47J | CEO | 19J | >0.2 to <0.5 | >85% | >105% |
| 47K | Comp | 19K | >0.2 to <0.5 | >85% | >105% |
| 47L | CEO | 19L | >0.2 to <0.5 | >85% | >105% |
| 47M | CEO | 19M | >0.2 to <0.5 | >85% | >105% |
| 47N | Comp | 19N | >0.2 to <0.5 | >85% | >105% |
| 47O | CEO | 19O | >0.2 to <0.5 | >85% | >105% |
| 47P | Comp | 19P | >0.2 to <0.5 | >85% | >105% |
| 47Q | CEO | 19Q | >0.2 to <0.5 | >85% | >105% |
| 47R | Comp | 19R | >0.2 to <0.5 | >85% | >105% |
| 47S | CEO | 19S | >0.2 to <0.5 | >85% | >105% |
| 47T | CEO | 19T | >0.2 to <0.5 | >85% | >105% |
| 47U | CEO | 20U | >0.2 to <0.5 | >85% | >105% |
| 48A | Comp | 20A | >0.2 to <0.5 | >85% | >105% |
| 48B | CEO | 20B | >0.2 to <0.5 | >85% | >105% |
| 48C | Comp | 20C | >0.2 to <0.5 | >85% | >105% |
| 48D | CEO | 20D | >0.2 to <0.5 | >85% | >105% |
| 48E | Comp | 20E | >0.2 to <0.5 | >85% | >105% |
| 48F | CEO | 20F | >0.2 to <0.5 | >85% | >105% |
| 48G | Comp | 20G | >0.2 to <0.5 | >85% | >105% |
| 48H | CEO | 20H | >0.2 to <0.5 | >85% | >105% |
| 48I | Comp | 20I | >0.2 to <0.5 | >85% | >105% |
| 48J | CEO | 20J | >0.2 to <0.5 | >85% | >105% |
| 48K | Comp | 20K | >0.2 to <0.5 | >85% | >105% |
| 48L | CEO | 20L | >0.2 to <0.5 | >85% | >105% |
| 48M | CEO | 20M | >0.2 to <0.5 | >85% | >105% |
| 48N | Comp | 20N | >0.2 to <0.5 | >85% | >105% |
| 48O | CEO | 20O | >0.2 to <0.5 | >85% | >105% |
| 48P | Comp | 20P | >0.2 to <0.5 | >85% | >105% |
| 48Q | CEO | 20Q | >0.2 to <0.5 | >85% | >105% |
| 48R | Comp | 20R | >0.2 to <0.5 | >85% | >105% |
| 48S | CEO | 20S | >0.2 to <0.5 | >85% | >105% |
| 48T | CEO | 20T | >0.2 to <0.5 | >85% | >105% |
| 48U | CEO | 21U | >0.2 to <0.5 | >85% | >105% |
| 49A | Comp | 21A | >0.2 to <0.5 | >85% | >105% |
| 49B | CEO | 21B | >0.2 to <0.5 | >85% | >105% |
| 49C | Comp | 21C | >0.2 to <0.5 | >85% | >105% |
| 49D | CEO | 21D | >0.2 to <0.5 | >85% | >105% |
| 49E | Comp | 21E | >0.2 to <0.5 | >85% | >105% |
| 49F | CEO | 21F | >0.2 to <0.5 | >85% | >105% |
| 49G | Comp | 21G | >0.2 to <0.5 | >85% | >105% |
| 49H | CEO | 21H | >0.2 to <0.5 | >85% | >105% |
| 49I | Comp | 21I | >0.2 to <0.5 | >85% | >105% |
| 49J | CEO | 21J | >0.2 to <0.5 | >85% | >105% |
| 49K | Comp | 21K | >0.2 to <0.5 | >85% | >105% |
| 49L | CEO | 21L | >0.2 to <0.5 | >85% | >105% |
| 49M | CEO | 21M | >0.2 to <0.5 | >85% | >105% |
| 49N | Comp | 21N | >0.2 to <0.5 | >85% | >105% |
| 49O | CEO | 21O | >0.2 to <0.5 | >85% | >105% |
| 49P | Comp | 21P | >0.2 to <0.5 | >85% | >105% |
| 49Q | CEO | 21Q | >0.2 to <0.5 | >85% | >105% |
| 49R | Comp | 21R | >0.2 to <0.5 | >85% | >105% |
| 49S | CEO | 21S | >0.2 to <0.5 | >85% | >105% |
| 49T | CEO | 21T | >0.2 to <0.5 | >85% | >105% |
| 49U | CEO | 21U | >0.2 to <0.5 | >85% | >105% |
| 50A | Comp | 22A | >0.2 to <0.5 | >85% | >105% |
| 50B | CEO | 22B | >0.2 to <0.5 | >85% | >105% |
| 50C | Comp | 22C | >0.2 to <0.5 | >85% | >105% |
| 50D | CEO | 22D | >0.2 to <0.5 | >85% | >105% |
| 50E | Comp | 22E | >0.2 to <0.5 | >85% | >105% |
| 50F | CEO | 22F | >0.2 to <0.5 | >85% | >105% |
| 50G | Comp | 22G | >0.2 to <0.5 | >85% | >105% |
| 50H | CEO | 22H | >0.2 to <0.5 | >85% | >105% |
| 50I | Comp | 22I | >0.2 to <0.5 | >85% | >105% |
| 50J | CEO | 22J | >0.2 to <0.5 | >85% | >105% |
| 50K | Comp | 22K | >0.2 to <0.5 | >85% | >105% |
| 50L | CEO | 22L | >0.2 to <0.5 | >85% | >105% |
| 50M | CEO | 22M | >0.2 to <0.5 | >85% | >105% |
| 50N | Comp | 22N | >0.2 to <0.5 | >85% | >105% |
| 50O | CEO | 22O | >0.2 to <0.5 | >85% | >105% |
| 50P | Comp | 22P | >0.2 to <0.5 | >85% | >105% |
| 50Q | CEO | 22Q | >0.2 to <0.5 | >85% | >105% |
| 50R | Comp | 22R | >0.2 to <0.5 | >85% | >105% |
| 50S | CEO | 22S | >0.2 to <0.5 | >85% | >105% |
| 50T | CEO | 22T | >0.2 to <0.5 | >85% | >105% |
| 50U | CEO | 22U | >0.2 to <0.5 | >85% | >105% |
| 51A | Comp | 23A | >0.2 to <0.5 | >85% | >105% |
| 51B | CEO | 23B | >0.2 to <0.5 | >85% | >105% |
| 51C | Comp | 23C | >0.2 to <0.5 | >85% | >105% |
| 51D | CEO | 23D | >0.2 to <0.5 | >85% | >105% |
| 51E | Comp | 23E | >0.2 to <0.5 | >85% | >105% |
| 51F | CEO | 23F | >0.2 to <0.5 | >85% | >105% |
| 51G | Comp | 23G | >0.2 to <0.5 | >85% | >105% |
| 51H | CEO | 23H | >0.2 to <0.5 | >85% | >105% |
| 51I | Comp | 23I | >0.2 to <0.5 | >85% | >105% |
| 51J | CEO | 23J | >0.2 to <0.5 | >85% | >105% |
| 51K | Comp | 23K | >0.2 to <0.5 | >85% | >105% |
| 51L | CEO | 23L | >0.2 to <0.5 | >85% | >105% |
| 51M | CEO | 23M | >0.2 to <0.5 | >85% | >105% |
| 51N | Comp | 23N | >0.2 to <0.5 | >85% | >105% |
| 51O | CEO | 23O | >0.2 to <0.5 | >85% | >105% |
| 51P | Comp | 23P | >0.2 to <0.5 | >85% | >105% |
| 51Q | CEO | 23Q | >0.2 to <0.5 | >85% | >105% |
| 51R | Comp | 23R | >0.2 to <0.5 | >85% | >105% |
| 51S | CEO | 23S | >0.2 to <0.5 | >85% | >105% |
| 51T | CEO | 23T | >0.2 to <0.5 | >85% | >105% |
| 51U | CEO | 23U | >0.2 to <0.5 | >85% | >105% |
| 52A | Comp | 24A | >0.2 to <0.5 | >85% | >105% |
| 52B | CEO | 24B | >0.2 to <0.5 | >85% | >105% |
| 52C | Comp | 24C | >0.2 to <0.5 | >85% | >105% |
| 52D | CEO | 24D | >0.2 to <0.5 | >85% | >105% |
| 52E | Comp | 24E | >0.2 to <0.5 | >85% | >105% |
| 52F | CEO | 24F | >0.2 to <0.5 | >85% | >105% |
| 52G | Comp | 24G | >0.2 to <0.5 | >85% | >105% |
| 52H | CEO | 24H | >0.2 to <0.5 | >85% | >105% |
| 52I | Comp | 24I | >0.2 to <0.5 | >85% | >105% |
| 52J | CEO | 24J | >0.2 to <0.5 | >85% | >105% |
| 52K | Comp | 24K | >0.2 to <0.5 | >85% | >105% |
| 52L | CEO | 24L | >0.2 to <0.5 | >85% | >105% |
| 52M | CEO | 24M | >0.2 to <0.5 | >85% | >105% |
| 52N | Comp | 24N | >0.2 to <0.5 | >85% | >105% |
| 52O | CEO | 24O | >0.2 to <0.5 | >85% | >105% |
| 52P | Comp | 24P | >0.2 to <0.5 | >85% | >105% |
| 52Q | CEO | 24Q | >0.2 to <0.5 | >85% | >105% |
| 52R | Comp | 24R | >0.2 to <0.5 | >85% | >105% |
| 52S | CEO | 24S | >0.2 to <0.5 | >85% | >105% |
| 52T | CEO | 24T | >0.2 to <0.5 | >85% | >105% |
| 52U | CEO | 24U | >0.2 to <0.5 | >85% | >105% |
| 53A | Comp | 25A | >0.2 to <0.5 | >85% | >105% |
| 53B | CEO | 25B | >0.2 to <0.5 | >85% | >105% |
| 53C | Comp | 25C | >0.2 to <0.5 | >85% | >105% |
| 53D | CEO | 25D | >0.2 to <0.5 | >85% | >105% |
| 53E | Comp | 25E | >0.2 to <0.5 | >85% | >105% |
| 53F | CEO | 25F | >0.2 to <0.5 | >85% | >105% |
| 53G | Comp | 25G | >0.2 to <0.5 | >85% | >105% |
| 53H | CEO | 25H | >0.2 to <0.5 | >85% | >105% |
| 53I | Comp | 25I | >0.2 to <0.5 | >85% | >105% |
| 53J | CEO | 25J | >0.2 to <0.5 | >85% | >105% |
| 53K | Comp | 25K | >0.2 to <0.5 | >85% | >105% |
| 53L | CEO | 25L | >0.2 to <0.5 | >85% | >105% |
| 53M | CEO | 25M | >0.2 to <0.5 | >85% | >105% |
| 53N | Comp | 25N | >0.2 to <0.5 | >85% | >105% |
| 53O | CEO | 25O | >0.2 to <0.5 | >85% | >105% |
| 53P | Comp | 25P | >0.2 to <0.5 | >85% | >105% |
| 53Q | CEO | 25Q | >0.2 to <0.5 | >85% | >105% |
| 53R | Comp | 25R | >0.2 to <0.5 | >85% | >105% |
| 53S | CEO | 25S | >0.2 to <0.5 | >85% | >105% |
| 53T | CEO | 25T | >0.2 to <0.5 | >85% | >105% |
| 53U | CEO | 25U | >0.2 to <0.5 | >85% | >105% |
| 54A | Comp | 26A | >0.2 to <0.5 | >85% | >105% |
| 54B | CEO | 26B | >0.2 to <0.5 | >85% | >105% |
| 54C | Comp | 26C | >0.2 to <0.5 | >85% | >105% |
| 54D | CEO | 26D | >0.2 to <0.5 | >85% | >105% |
| 54E | Comp | 26E | >0.2 to <0.5 | >85% | >105% |
| 54F | CEO | 26F | >0.2 to <0.5 | >85% | >105% |
| 54G | Comp | 26G | >0.2 to <0.5 | >85% | >105% |
| 54H | CEO | 26H | >0.2 to <0.5 | >85% | >105% |
| 54I | Comp | 26I | >0.2 to <0.5 | >85% | >105% |
| 54J | CEO | 26J | >0.2 to <0.5 | >85% | >105% |
| 54K | Comp | 26K | >0.2 to <0.5 | >85% | >105% |
| 54L | CEO | 26L | >0.2 to <0.5 | >85% | >105% |
| 54M | CEO | 26M | >0.2 to <0.5 | >85% | >105% |
| 54N | Comp | 26N | >0.2 to <0.5 | >85% | >105% |
| 54O | CEO | 26O | >0.2 to <0.5 | >85% | >105% |
| 54P | Comp | 26P | >0.2 to <0.5 | >85% | >105% |
| 54Q | CEO | 26Q | >0.2 to <0.5 | >85% | >105% |
| 54R | Comp | 26R | >0.2 to <0.5 | >85% | >105% |
| 54S | CEO | 26S | >0.2 to <0.5 | >85% | >105% |
| 54T | CEO | 26T | >0.2 to <0.5 | >85% | >105% |
| 54U | CEO | 26U | >0.2 to <0.5 | >85% | >105% |

The present invention thus provides pressurized metered dose inhalers (MDIs), including each of MDI1 through MDI54, for the treatment of asthma and other chronic obstructive pulmonary diseases or other diseases as are known to be treatable by those skilled in the art by ipratropium by delivery thereof to the lungs of the patient.

The present invention thus includes methods for delivering of pharmaceutical compositions, including Pharmaceutical Delivery Methods 1 through 6, for purpose of treating ailments, diseases and similar health related problems of an organism (such as a human or animal) comprising applying a composition of the present invention containing ipratropium, including any and all pharmaceutically effective salts thereof, to the organism in need of treatment.

The MDI metering valve size, that is, the size of the metering chamber, can vary within the scope hereof, but may be between 10 microliters (μL or mcl) and 100 microliters, or from about 25 to about −90, or from about 40 microliters to about 80 microliters, or from about 50 to about 70, or from about 60 to about 65 microliters.

In certain embodiments, pMDIs of the present invention, including each of MDI1 through MDI54 and methods of the present disclosure use a pMDI of the present invention, deliver API in an amount of from about 10 to about 100 milligram per actuation (mg/actuation), or about 10 to about 50 milligram per actuation (mg/actuation), or about 20 to about 30 milligram per actuation (mg/actuation).

In certain embodiments, pMDIs of the present invention, including each of MDI1 through MDI54 and methods of the present disclosure use a pMDI of the present invention, deliver an aerosol spray having a fine particle fraction that is greater than 30%.

In certain embodiments, pMDIs of the present invention, including each of MDI1 through MDI54 and methods of the present disclosure use a pMDI of the present invention, deliver an aerosol spray having a fine particle fraction that is greater than 35%.

In certain embodiments, pMDIs of the present invention, including each of MDI1 through MDI54 and methods of the present disclosure use a pMDI of the present invention, deliver an aerosol spray having a fine particle fraction that is greater than 50%.

In certain embodiments, pMDIs of the present invention, including each of MDI1 through MDI54 and methods of the present disclosure use a pMDI of the present invention, deliver an aerosol spray having a fine particle fraction that is greater than 60%.

In certain embodiments, pMDIs of the present invention, including each of MDI1 through MDI54 and methods of the present disclosure use a pMDI of the present invention, deliver an aerosol spray having a fine particle fraction that is greater than 70%.

In certain embodiments, pMDIs of the present invention, including each of MDI1 through MDI54 and methods of the present disclosure use a pMDI of the present invention, deliver an aerosol spray having a fine particle fraction that is greater than 80%.

In certain embodiments, pMDIs of the present invention, including each of MDI1 through MDI54 and methods of the present disclosure use a pMDI of the present invention, contain a formulation in which the API comprises, consists essentially of or consists of beclomethasone, has a orifice diameter of less than 0.3 mm, and deliver an aerosol spray having a fine particle fraction that is greater than 65%.

In certain embodiments, pMDIs of the present invention, including each of MDI1 through MDI54 and methods of the present disclosure use a pMDI of the present invention, contain a formulation in which the API comprises, consists essentially of or consists of beclomethasone, has a orifice diameter of less than 0.25 mm, and deliver an aerosol spray having a fine particle fraction that is greater than 65%.

In certain embodiments, pMDIs of the present invention, including each of MDI1 through MDI54 and methods of the present disclosure use a pMDI of the present invention, contain a formulation in which the API comprises, consists essentially of or consists of formotorol, has a orifice diameter of less than 0.3 mm, and delivers an aerosol spray having a fine particle fraction that is greater than 30%.

In certain embodiments, pMDIs of the present invention, including each of MDI1 through MDI54 and methods of the present disclosure use a pMDI of the present invention, contain a formulation in which the API comprises, consists essentially of or consists of beclomethasone, have a orifice diameter of less than 0.25 mm, and deliver an aerosol spray having a fine particle fraction that is greater than 30%.

In certain embodiments, pMDIs of the present invention, including each of MDI1 through MDI54 and methods of the present disclosure use a pMDI of the present invention, contain a formulation in which the API comprises, consists essentially of or consists of glycopyrroniuml, has a orifice diameter of less than 0.3 mm, and delivers an aerosol spray having a fine particle fraction that is greater than 35%.

In certain embodiments, pMDIs of the present invention, including each of MDI1 through MDI54 and methods of the present disclosure use a pMDI of the present invention, contain a formulation in which the API comprises, consists essentially of or consists of beclomethasone, have a orifice diameter of less than 0.25 mm, and deliver an aerosol spray having a fine particle fraction that is greater than 50%.

The present invention includes methods of forming pharmaceutical compositions, including each of Pharmaceutical Compositions 1-3 and PC 4-9 and forming an aerosol comprising ipratropium bromide by forcing said pharmaceutical composition through an orifice having a diameter of less than 0.5 mm.

The present invention includes methods of forming pharmaceutical compositions, including each of Pharmaceutical Compositions 1-3 and PC 4-9 and forming an aerosol comprising ipratropium bromide by forcing said pharmaceutical composition through an orifice having a diameter of from about 0.2 mm to about 0.45. The present invention includes methods of forming pharmaceutical compositions, including each of Pharmaceutical Compositions 1-3 and PC 1-26 and forming an aerosol comprising ipratropium bromide by forcing said pharmaceutical composition through an orifice having a diameter of from about 0.2 mm to about 0.4 mm.

The present invention includes methods of forming pharmaceutical compositions, including each of Pharmaceutical Compositions 1-5 and PC 1-26 and forming an aerosol comprising API by forcing said pharmaceutical composition through an orifice having a diameter of from about 0.2 mm to about 0.35 mm.

The present invention includes methods of forming pharmaceutical compositions, including each of Pharmaceutical Compositions 1-5 and PC 1-26 and forming an aerosol comprising API by forcing said pharmaceutical composition through an orifice having a diameter of from about 0.2 mm to about 0.30 mm.

The present invention includes methods of forming pharmaceutical compositions, including each of Pharmaceutical Compositions 1-5 and PC 1-26 and forming an aerosol comprising API by forcing said pharmaceutical composition through an orifice having a diameter of from about 0.2 mm to 0.29 mm.

### EXAMPLES

### Comparative Example 1A—Formulation of Beclomethasone, HFC-134a and Ethanol

Beclomethasone dipropionate (BDP) as the API for the example is procured from MilliporeSigma (St Louis, MO, USA). Propellant grade HFC-134a is supplied by Honeywell (Morris Plains, NJ, USA). USP grade absolute ethanol is purchased from Fisher Scientific (Waltham, MA, USA). A BDP solution in ethanol is weighed directly into a suitable canister. A quantitative amount of ethanol in the final solution (about 10 wt % ethanol and 90% HFC-134a in the final formulation on the basis of ethanol and HFC-134a, which is reflective of a commonly available commercial formulation) is dispensed into the canister, and the canisters were fitted with metering valves as identified below in Comparative Example 2. Sealed canisters are then pressure-filled with HFC-134a propellant. Canisters are agitated to ensure adequate mixing and then left to equilibrate under 2-week quarantine.

### Comparative Example 1B—Formulation of Formoterol, HFC-134a and Ethanol

Formoterol formate (FF) as the API for the example is procured. Propellant grade HFC-134a is supplied by Honeywell (Morris Plains, NJ, USA). USP grade absolute ethanol is purchased from Fisher Scientific (Waltham, MA, USA). A FF solution in ethanol is weighed directly into a suitable canister. A quantitative amount of ethanol in the final solution (about 16 wt % ethanol and 84% HFC-134a in the final formulation on the basis of ethanol and HFC-134a in the final formulation, which is reflective of a commonly available commercial formulation) is dispensed into the canister, and the canisters are fitted with metering valves as identified below in Comparative Example 2. Sealed canisters are then pressure-filled with HFC-134a propellant. Canisters are agitated to ensure adequate mixing and then left to equilibrate under 2-week quarantine.

### Comparative Example 1C—Formulation of Glycopyrrolate, HFC-134a and Ethanol

Glycopyrrolate bromide (GB) as the API for the example is procured. Propellant grade HFC-134a is supplied by Honeywell (Morris Plains, NJ, USA). USP grade absolute ethanol is purchased from Fisher Scientific (Waltham, MA, USA). A GB solution in ethanol is weighed directly into a suitable canister. A quantitative amount of ethanol in the final solution (about 16 wt % ethanol and 84% HFC-134a in the final formulation on the basis of ethanol and HFC-134a, which is reflective of a commonly available commercial formulation) is dispensed into the canister, and the canisters are fitted with metering valves as identified below in Comparative Example 2. Sealed canisters are then pressure-filled with HFC-134a propellant. Canisters are agitated to ensure adequate mixing and then left to equilibrate under 2-week quarantine.

### Comparative Example 1D—Formulation of Ipratropium, HFC-134a and Ethanol

Ipratropium bromide (IB) as the API for the example is procured from MilliporeSigma (St Louis, MO, USA). Propellant grade HFC-134a is supplied by Honeywell (Morris Plains, NJ, USA). USP grade absolute ethanol was purchased from Fisher Scientific (Waltham, MA, USA). An IB solution in ethanol, water and citric acid is weighed directly into a suitable canister. A quantitative amount of ethanol in the final solution (about 15 wt % ethanol and about 0.5% water, 0.011 citric acid and the balance HFC-134a in the final formulation, which is reflective of a commonly available commercial formulation) is dispensed into the canister, and the canisters are fitted with metering valves as identified below in Comparative Example 3. Sealed canisters are then pressure-filled with HFC-134a propellant. Canisters are agitated to ensure adequate mixing and then left to equilibrate under 2-week quarantine.

### Comparative Example 2A, 2B and 2C—Delivered Dose Percentage for Formulations C1A, C1B and C1C Using 0.3 mm Actuator Orifice

The pMDI canisters of Comparative Example 1A, 1B and 1C are fitted onto 63 μl valves, and the valved cannisters are then mounted in a first series of actuators having a 0.3 mm diameter orifice to produce a pMDI generally as described above in connection FIGS. 1-3. Each value of DD reported herein was obtained by sampling 15 consecutive doses at a sampling flow rate of 28.3 l/minute (1SCFM). For each canister tested, the target does and delivered dose was determined using DUSA methodology (Dose Unit Spray Apparatus) at the beginning of the canister life, and this result is reported in Table CEx2 below. The DD % dosages are also selectively sampled for middle and end of canister-use life, and the result were found to be consistent with the beginning DD %. In this example, the DD % is determined by dividing the total amount of the composition leaving the actuator by the total amount of the composition leaving the valve.

The beginning DD % data for each pMDI is provided in Table CEx2 below:

TABLE CEx2

| Example | Formulation | API | DD % |
|---|---|---|---|
| ExC2A | CE1A | BDP | 76.3 |
| ExC2B | CE1B | FF | 92.9 |
| ExC2C | CE1C | GB | 65.7 |

### Example 1A—Formulation of Beclomethasone, HFO-1234ze(E) and Ethanol

Comparative Example 1A is repeated except that the HFC-134a is replaced by HFC-1234ze(E) on a weight basis (referred to hereinafter as Formulation 1A.)

### Example 1B—Formulation of Formoterol, HFO-1234ze(E) and Ethanol

Comparative Example 1B is repeated except that the HFC-134a is replaced by HFC-1234ze(E) on a weight basis (referred to hereinafter as Formulation 1B).

### Example 1C—Formulation of Glycopyrrolate, HFO-1234ze(E) and Ethanol

Comparative Example 1C is repeated except that the HFC-134a is replaced by HFC-1234ze(E) on a weight basis (referred to hereinafter as Formulation 1C).

### Example 1D—Formulation of Ipratropium, HFO-1234ze(E) and Ethanol

Comparative Example 1D is repeated except that the HFC-134a is replaced by HFC-1234ze(E) on a weight basis (referred to hereinafter as Formulation 1D).

### Example 2A, 2B and 2C—Delivered Dose Percentage for Formulations 1A, 1B and 1C Using 0.3 mm Actuator Orifice

Comparative Examples C1A, C1B and C1C are repeated, except that Formulations 1A, 1B and 1C are used on an equal weight basis in place of formulations C1A, C1B and C1C. For each canister tested, the target dose and delivered dose was determined using DUSA methodology (Dose Unit Spray Apparatus) at the beginning of the canister life, and this result is reported in Table Ex2 below. The DD % is also selectively determined for middle and end of canister-use life, and the result are found to be consistent with the beginning DD %. In this example, the DD % is determined by dividing the total amount of the composition leaving the actuator by the total amount of the composition leaving the valve. For ease of comparison, the results from Comparative Examples C1A, C1B and C1C are also provided in Table Ex2, together with the result being reported on a relative weight basis compared to the result using HFC-134a as in the comparative examples.

TABLE Ex2

| | | | | DD % |
|---|---|---|---|---|
| Example | Formulation | API | DD % | Relative 134a |
| ExC2A | C1A (10% | BDP | 76.3 | 100 |
| | ethanol; 90% | | | |
| | HFC-134a) | | | |
| Ex2A | 1A (10% ethanol; | BDP | 101.1 | 132.5 |
| | 90% 1234ze(E)) | | | |
| ExC2A | C1B (16% | FF | 92.9 | 100 |
| | ethanol; 84% | | | |
| | HFC-134a) | | | |
| Ex2B | 1B (16% | FF | 107.8 | 116.0 |
| | ethanol; 84% | | | |
| | 1234ze(E)) | | | |
| ExC2A | C1A (16% | GB | 65.7 | 100 |
| | ethanol; 84% | | | |
| | HFC-134a) | | | |
| Ex2C | 1C (16% ethanol; | GB | 98.5 | 149.9 |
| | 84% 1234ze(E)) | | | |

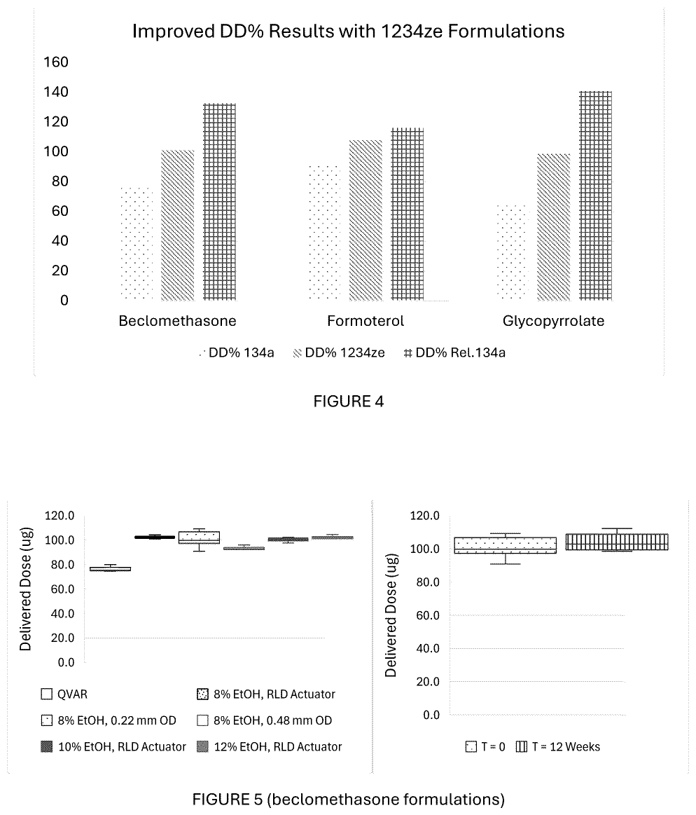

The results show DD % results that are unexpectedly and advantageously high. These DD % results, together with the results relative to the base-line performance using HFC-134a as reported in Table Ex2 above, are illustrated in FIG. 4.

### Example 3A1—Delivered Dose Percentage for Beclomethasone Formulation Containing 8% Ethanol and 92% 1234ze

Example 1A is repeated, except that the formulation is adjusted to contain 8% ethanol and 92% 1234ze (E). The DD % is about 98%, which is also unexpectedly and advantageously high.

### Example 3A2—Delivered Dose Percentage for Beclomethasone Formulation Containing 12% Ethanol and 88% 1234ze

Example 1A is repeated, except that the formulation contains 12% ethanol and 88% 1234ze (E). The DD % is about 101.1%, which is the same as Example 1A and also unexpectedly and advantageously high.

### Example 3A3—Delivered Dose Percentage for Beclomethasone Formulation Containing 8% Ethanol and 92% 1234ze Using an Actuator Orifice Diameter of about 0.5

Example 3A1 is repeated, except that the actuator orifice diameter is 0.48 mm. The DD % is about 91.2%, which is also unexpectedly and advantageously high.

### Example 3A4—Delivered Dose Percentage for Beclomethasone Formulation Containing 8% Ethanol and 92% 1234ze Using an Actuator Orifice Diameter of about 0.2

Example 3A1 is repeated, except that the actuator orifice diameter is 0.22 mm. The DD % is about 102.9%, which is also unexpectedly and advantageously high.

### Example 3B1—Delivered Dose Percentage for Formoterol Formulation Containing 15% Ethanol and 85% 1234ze

Example 1B is repeated, except that the formulation is adjusted to contain about 15% ethanol and 85% 1234ze (E). The DD % is about 100%, which is which is also unexpectedly and advantageously high.

### Example 3B2—Delivered Dose Percentage for Formoterol Formulation Containing 18% Ethanol and 82% 1234ze

Example 1B is repeated, except that the formulation is adjusted to contain about 18% ethanol and 82% 1234ze (E). The DD % is about 109.5%, which is also unexpectedly and advantageously high.

### Example 3B3—Delivered Dose Percentage for Formoterol Formulation Containing 18% Ethanol and 82% 1234ze Using an Actuator Orifice Diameter of about 0.5

Example 3B2 is repeated, except that the actuator orifice diameter is 0.48 mm. The DD % is about 106.2%, which is also unexpectedly and advantageously high.

### Example 3B4—Delivered Dose Percentage for Formoterol Formulation Containing 18% Ethanol and 82% 1234ze Using an Actuator Orifice Diameter of about 0.2

Example 3B2 is repeated, except that the actuator orifice diameter is 0.22 mm. The DD % is about 101.5%, which is also unexpectedly and advantageously high.

### Example 3C1—Delivered Dose Percentage for Glycopyrrolate Formulation Containing 12.5% Ethanol and 87.5% 1234ze

Example 1C is repeated, except that the formulation is adjusted to contain about 15% ethanol and 85% 1234ze (E). The DD % is about 100%, which is also unexpectedly and advantageously high.

### Example 3C2—Delivered Dose Percentage for Glycopyrrolate Formulation Containing 20% Ethanol and 80% 1234ze

Example 3C1 is repeated, except that except that the formulation is adjusted to contain about 20.3% ethanol and 89.7% 1234ze (E). The DD % is about 82.3%, which is also unexpectedly and advantageously high.

### Example 3C3—Delivered Dose Percentage for Glycopyrrolate Formulation Containing 12.5% Ethanol and 87.5% 1234ze Using an Actuator Orifice Diameter of about 0.5

Example 3C1 is repeated, except that the actuator orifice diameter is 0.48 mm. The DD % is about 74.6%, which is also unexpectedly and advantageously high.

### Example 3C4—Delivered Dose Percentage for Glycopyrrolate Formulation Containing 12.5% Ethanol and 87.5% 1234ze Using an Actuator Orifice Diameter of about 0.2

Example 3C1 is repeated, except that the actuator orifice diameter is 0.22 mm. The DD % is about 74.0%, which is also unexpectedly and advantageously high.

### Example 4A—Delivered Dose Percentage for Formulation 1D Using 0.3 mm Actuator Orifice

Comparative Example C1D is repeated, except that Formulation 1D is used on an equal weight basis in place of formulation C1D. For each canister tested, the target dose and delivered dose was determined using DUSA methodology (Dose Unit Spray Apparatus) at the beginning of the canister life, and this result is reported in Table Ex4 below. The DD % is also selectively determined for middle and end of canister-use life, and the result are found to be consistent with the beginning DD %. In this example, the DD % is determined by dividing the total amount of the composition leaving the actuator by the total amount of the composition leaving the valve. The DD % result is unexpectedly and advantageously high.

### Examples 5A-5C—Fine Particle Test Results and Additional Data

The spray formulations of each of Examples 2, 3 and 4 were tested to determine aerodynamic particle size distribution (APSD) with n=3.

### 5A-Beclomethasone

For the present beclomethasone formulations, fine particle dose (FPD≤5 μm) through a QVAR brand pMDI with a 0.3 mm orifice diameter had significantly higher fine particle fraction (FPF≤5 μm) (see Table Ex5A) than the comparative formulation, which is the current registered listed drug (“RLD”), through the same QVAR pMDI (compare the results in column 2 in Table Ex5A below to column 3). This result is an advantageous and unexpected result. In a further unexpected result, reducing the orifice diameter to 0.22 mm using the present formulation with 8% ethanol and 92% HFO1234zeE, surprising caused a near doubling of the FPF compared to the test results using a 0.3 mm orifice diameter (compare 44% (column 3) to 80.2 (column 4). Increasing ethanol content across the test formulations slightly decreased FPD. Mass median aerodynamic diameter (MMAD) showed a statistical increase (p<0.05) while geometric standard deviation (GSD) did not statistically change (p>0.1) when compared to the RLD. Most FPD loss is from increased throat deposition. Storage under ICH accelerated conditions (40° C., 75% RH) for 12 weeks (aged formulations) saw little APSD change. The results are reported in Table Ex5A below and illustrated graphically in FIG. 5 (the box in the figure represents the interquartile range while the whiskers represent the minimum and maximum).

TABLE 5A
APSD for QVAR and beclomethasone Test formulations in HFO-1234ze(E)

| | | 8% | 8% | 8% | 10% | 12% | 8% |
|---|---|---|---|---|---|---|---|
| | QVAR | EtOH | EtOH | EtOH | EtOH | EtOH | EtOH |
| | Actuator | | | | | | |
| | RLD, | RLD, | | | RLD, | RLD, | |
| | 0.3 mm | 0.3 mm | 0.22 mm | 0.48 mm | 0.3 mm | 0.3 mm | 0.22 mm |
| | OD | OD | OD | OD | OD | OD | OD |
| | Time | | | | | | |
| | 0 | 0 | 0 | 0 | 0 | 0 | 12 weeks |
| Throat | 26.8 | 46.6 | 14.1 | 65.9 | 52.0 | 58.0 | 17.0 |
| Stage 1 | 3.2 | 0.6 | 0.8 | 0.7 | 1.0 | 1.4 | 0.7 |
| Stage 2 | 0.5 | 0.2 | 0.3 | 0.2 | 0.3 | 0.3 | 0.1 |
| Stage 3 | 0.3 | 0.6 | 0.5 | 0.5 | 0.6 | 0.5 | 0.3 |
| Stage 4 | 1.9 | 4.9 | 4.2 | 4.3 | 4.6 | 3.6 | 2.1 |
| Stage 5 | 8.8 | 13.5 | 22.2 | 10.9 | 12.7 | 9.7 | 17.1 |
| Stage 6 | 14.7 | 13.1 | 26.9 | 10.1 | 12.4 | 10.7 | 29.3 |
| Stage 7 | 7.5 | 7.5 | 15.9 | 6.0 | 6.5 | 4.5 | 19.6 |
| Stage 8 | 6.7 | 4.8 | 10.7 | 4.1 | 4.4 | 3.7 | 9.2 |
| Sum | 70.2 | 91.8 | 95.7 | 102.7 | 94.4 | 92.3 | 95.4 |
| FPD ≤5 μm (μg) | 39.6 | 44.1 | 80.2 | 35.6 | 40.8 | 32.4 | 77.4 |
| FPF (FPD as % | 56.6 | 48.0 | 83.7 | 35.1 | 43.2 | 35.2 | 81.4 |
| MMAD (μm) | 1.1 | 1.3 | 1.1 | 1.3 | 1.3 | 1.3 | 1.0 |
| GSD | 2.2 | 1.8 | 1.7 | 1.8 | 1.8 | 1.9 | 1.6 |

### 5B—Formoterol

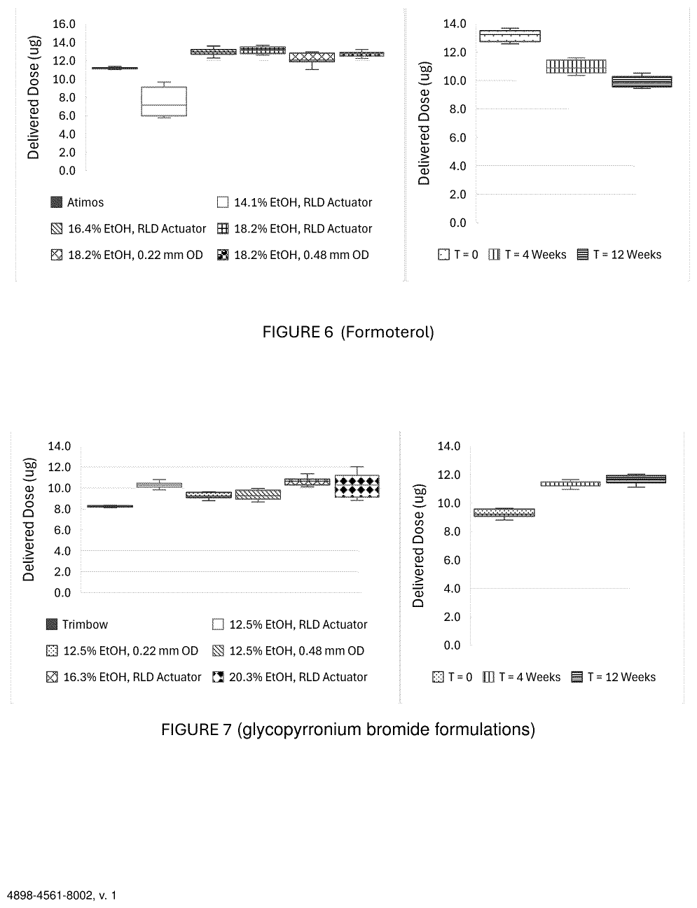

For comparative purposes, the RLD ATIMOS in the brand actuator was tested (see column 2 in Table Ex5B below). The same actuator used for the RLD with the same orifice diameter (0.4 mm) was used for the present formoterol formulations (see columns 3, 4 and 5) with ethanol concentrations of 14.1%, 16.4% and 18.2% by weight. These results showed similar FPD to the RLD, though with larger MMADs. The 18.2% ethanol formulations were then tested in the same actuator but with two different orifice diameters, namely, 0.22 mm and 0.48 mm. The use of the 0.22 mm OD drastically and unexpectedly increased the FPD (compare column 6 (43.4% FPF) to column 5 (16.2% with 0.4 OC), and lowered the MMAD to be in the same range as the RLD. The results are reported in Table Ex5B below and illustrated graphically in FIG. 6 (the box in the figure represents the interquartile range while the whiskers represent the minimum and maximum).

TABLE Ex5B

| | | 14.1% | 16.4% | 18.2% | 18.2% | 18.2% | 18.2% | 18.2% |
|---|---|---|---|---|---|---|---|---|
| | Atimos | EtOH | EtOH | EtOH | EtOH | EtOH | EtOH | EtOH |
| | Actuator | | | | | | | |
| | RLD, | RLD, | RLD, | RLD, | | | | |
| | 0.4 mm | 0.4 mm | 0.4 mm | 0.4 mm | 0.22 mm | 0.48 mm | 0.22 mm | 0.22 mm |
| | OD | OD | OD | OD | OD | OD | OD | OD |
| | Time | | | | | | | |
| | 0 | 0 | 0 | 0 | 0 | 0 | 4 weeks | 12 weeks |
| Throat | 13.2 | 6.2 | 8.0 | 9.7 | 6.3 | 10.9 | 6.0 | 5.3 |
| Stage 1 | 0.2 | 0.2 | 1.9 | 0.4 | 0.2 | 0.2 | 0.2 | 0.1 |
| Stage 2 | 0.0 | 0.0 | 0.0 | 0.1 | 0.1 | 0.0 | 0.1 | 0.1 |
| Stage 3 | 0.0 | 0.0 | 0.0 | 0.0 | 0.0 | 0.0 | 0.1 | 0.0 |
| Stage 4 | 0.0 | 0.1 | 0.0 | 0.1 | 0.1 | 0.0 | 0.1 | 0.1 |
| Stage 5 | 0.2 | 0.4 | 0.3 | 0.5 | 0.5 | 0.3 | 0.6 | 0.3 |
| Stage 6 | 1.0 | 0.6 | 0.4 | 0.6 | 1.5 | 0.5 | 1.3 | 1.0 |
| Stage 7 | 1.0 | 0.5 | 0.4 | 0.4 | 1.4 | 0.4 | 1.0 | 1.1 |
| Stage 8 | 1.8 | 0.4 | 0.2 | 0.3 | 1.5 | 0.3 | 0.6 | 0.8 |
| Sum | 17.4 | 8.4 | 11.3 | 12.1 | 11.7 | 12.6 | 10.0 | 8.7 |
| FPD ≤5 μm | 2.0 | 1.9 | 1.3 | 2.0 | 5.1 | 1.5 | 3.6 | 3.2 |
| (μg) | | | | | | | | |
| FPF (FPD as | 23.1 | 22.8 | 11.9 | 16.2 | 43.4 | 11.7 | 36.2 | 37.1 |
| % total dose) | | | | | | | | |
| MMAD (μm) | 0.6 | 1.0 | N/A | 1.2 | 0.8 | 1.0 | 1.0 | 0.8 |
| GSD | 1.8 | 2.2 | N/A | N/A | 1.8 | 2.6 | 2.0 | 1.7 |

### 5C—Glycopyrronium Bromide

For comparative purposes, the TRIMBOW RLD actuator was tested with the RLD formulation containing glycopyrronium bromide as well as beclomethasone, and formoterol (see column 2 in Table Ex5C below). The same actuator used for the RLD with the same orifice diameter (0.3 mm) was used for the present glycopyrronium formulations (see columns 3, 5 and 6) with ethanol concentrations of 12.5%, 16.3% and 20.3% by weight. For the present glycopyrronium bromide formulations, APSD testing of the lower ethanol concentration test formulation provided a similar FPD compared to the RLD while the others gave decreasing FPDs with increasing ethanol concentrations (Table 5C). The MMAD of the test formulations were smaller, though comparison to the RLD is difficult since it is a combination product. Using a 0.22 mm OD in the actuator increased the FPD drastically and unexpectedly compared to the RLD actuator. After aging at standard conditions, there was no significant change in APSD at 4 or 12 weeks. The results are reported in Table Ex5C below and illustrated graphically in FIG. 7 (the box in the figure represents the interquartile range while the whiskers represent the minimum and maximum.

TABLE Ex5C

| | | 12.5% | 12.5% | 16.3% | 20.3% | 12.5% | 12.5% |
|---|---|---|---|---|---|---|---|
| | Trimbow | EtOH | EtOH | EtOH | EtOH | EtOH | EtOH |
| | Actuator | | | | | | |
| | RLD, | RLD, | | RLD, | RLD, | | |
| | 0.3 mm | 0.3 mm | 0.22 mm | 0.3 mm | 0.3 mm | 0.22 mm | 0.22 mm |
| | OD | OD | OD | OD | OD | OD | OD |
| | Time | | | | | | |
| | 0.0 | 0.0 | 0.0 | 0.0 | 0.0 | 4 weeks | 12 weeks |
| Throat | 5.4 | 7.4 | 3.1 | 7.6 | 10.0 | 5.3 | 4.0 |
| Stage 1 | 0.1 | 0.1 | 0.1 | 0.2 | 0.2 | 0.1 | 0.3 |
| Stage 2 | 0.0 | 0.0 | 0.0 | 0.0 | 0.0 | 0.1 | 0.1 |
| Stage 3 | 0.0 | 0.0 | 0.0 | 0.0 | 0.0 | 0.0 | 0.0 |
| Stage 4 | 0.3 | 0.0 | 0.0 | 0.0 | 0.0 | 0.0 | 0.1 |
| Stage 5 | 0.9 | 0.5 | 0.6 | 0.4 | 0.2 | 0.3 | 0.8 |
| Stage 6 | 0.9 | 0.8 | 1.6 | 0.6 | 0.4 | 1.3 | 2.1 |
| Stage 7 | 0.4 | 0.6 | 1.7 | 0.4 | 0.3 | 1.8 | 1.9 |
| Stage 8 | 0.2 | 0.5 | 1.0 | 0.4 | 0.3 | 1.5 | 1.6 |
| Sum | 8.2 | 9.8 | 8.1 | 9.5 | 11.6 | 10.4 | 10.8 |
| FPD ≤5 μm (μg) | 2.7 | 2.3 | 4.9 | 1.7 | 1.3 | 4.9 | 6.5 |
| FPF (FPD as % | 32.7 | 23.5 | 60.4 | 17.5 | 11.6 | 46.8 | 59.6 |
| total dose) | | | | | | | |
| MMAD (μm) | 1.4 | 1.0 | 0.8 | 1.0 | 1.0 | 0.7 | 0.9 |
| GSD | 1.8 | 1.8 | 1.7 | 2.0 | N/A | 1.7 | 1.8 |

What is claimed is:

1. A method for delivering to the mouth a user a dose of a pharmaceutical formulation at a high percentage of the targeted dose of the pharmaceutical formulation comprising:
a. providing a pMDI comprising:
i. a canister;
ii. a pharmaceutical formulation in the canister and comprising: (1) from about 75% by weight to about 95% by weight of HFO-1234ze(E); (2) from about 5% by weight to about 25% by weight of ethanol; and (3) an API in solution in one or more of said HFO-1234ze(E) and said ethanol; and
iii. a metering valve releasably closing the canister;
iv. an actuator fluidly connected to the metering valve and having an orifice diameter of about 0.6 mm or less; and
b. actuating said pMDI to introduce into the mouth of the user an aerosol spray formed from said pharmaceutical formulation and containing said API, wherein the amount of said aerosol spray introduced into the mouth of the user is greater than about 80% of said targeted dose.

2. The method of claim 1 wherein the amount of said aerosol spray introduced into the mouth of the user has a fine particle fraction of at least 30%.

3. The method of claim 1 wherein the amount of said aerosol spray introduced into the mouth of the user is greater than about 90% of said targeted dose.

4. The method of claim 2 wherein the amount of said aerosol spray introduced into the mouth of the user is greater than about 95% of said targeted dose.

5. The method of claim 1 wherein the metering valve has an orifice diameter of about 0.5 mm or less.

6. The method of claim 1 wherein said pharmaceutical formulation comprises: (1) from about 75% by weight to about 90% by weight of HFO-1234ze(E); (2) from about 10% by weight to about 25% by weight of ethanol.

7. The method of claim 5 wherein said pharmaceutical formulation comprises: (1) from about 80% by weight to about 90% by weight of HFO-1234ze(E); (2) from about 10% by weight to about 20% by weight of ethanol.

8. The method of claim 1 wherein said API is selected from the group consisting of ipratropium, beclomethasone, formoterol, glycopyrrolate and combinations of two or more of these.

9. The method of claim 1 wherein said API comprises ipratropium.

10. The method of claim 1 wherein said API comprises beclomethasone.

11. The method of claim 1 wherein said API comprises formoterol.

12. The method of claim 1 wherein said API comprises glycopyrrolate.

13. The method of claim 1 wherein the amount of said aerosol spray introduced into the mouth of the user is at least about 105% of the amount of said aerosol spray introduced into the mouth of the user when HFC-134a is used in place of said HFC-1234ze(E) on a weight for weight basis in said formulation.

14. The method of claim 13 wherein the said aerosol spray has a fine particle fraction that is at least about 35%.

15. The method of claim 3 wherein said aerosol spray has a fine particle fraction that is at least about 50%.

16. The method of claim 1 wherein the amount of said aerosol spray introduced into the mouth of the user is at least about 125% of the amount of said aerosol spray introduced into the mouth of the user when HFC-134a is used in place of said HFC-1234ze(E) on a weight for weight basis in said formulation.

17. The method of claim 1 wherein said actuator has an orifice diameter of less than 0.4 mm and wherein said API comprises ipratropium bromide.

18. The method of claim 1 wherein said actuator has an orifice diameter of less than 0.4 mm and wherein said API comprises beclomethasone dipropionate.

19. The method claim 1 wherein said actuator has an orifice diameter of less than 0.4 mm and wherein said API comprises formoterol fumarate.

20. The method of claim 1 wherein said actuator has an orifice diameter of less than 0.4 mm and wherein said API comprises glycopyrrolate bromide.
